SEQUENCE LISTING (68 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 292 aa, Severe acute respiratory syndrome coronavirus 2
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKS

SEQ ID NO: 3 - protein, 223 aa, Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 4 - protein, 19 aa, Severe acute respiratory syndrome coronavirus 2
IYKTPPIKDFGGFNFSQIL

SEQ ID NO: 5 - protein, 18 aa, Severe acute respiratory syndrome coronavirus 2
SFIEDLLFNKVTLADAGF

SEQ ID NO: 6 - protein, 73 aa, Severe acute respiratory syndrome coronavirus 2
TQNVLYENQKLIANQFNSAIGKIQDSLSSTASALGKLQDVVNQNAQALNTLVKQLSSNFGAISSVLNDILSRL

SEQ ID NO: 7 - protein, 50 aa, Severe acute respiratory syndrome coronavirus 2
KVEAEVQIDRLITGRLQSLQTYVTQQLIRAAEIRASANLAATKMSECVLG

SEQ ID NO: 8 - protein, 66 aa, Severe acute respiratory syndrome coronavirus 2
TTAPAICHDGKAHFPREGVFVSNGTHWFVTQRNFYEPQIITTDNTFVSGNCDVVIGIVNNTVYDPL

SEQ ID NO: 9 - protein, 51 aa, Severe acute respiratory syndrome coronavirus 2
DVDLGDISGINASVVNIQKEIDRLNEVAKNLNESLIDLQELGKYEQYIKWP

SEQ ID NO: 10 - protein, 24 aa, Severe acute respiratory syndrome coronavirus 2
WYIWLGFIAGLIAIVMVTIMLCCM

SEQ ID NO: 11 - protein, 36 aa, Severe acute respiratory syndrome coronavirus 2
TSCCSCLKGCCSCGSCCKFDEDDSEPVLKGVKLHYT

SEQ ID NO: 12 - DNA, 3822 bp, Severe acute respiratory syndrome coronavirus 2
  source 1..3822: mol_type = genomic DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 13 - protein, 10 aa, Human immunodeficiency virus 1
KRWIILGLNK

SEQ ID NO: 14 - protein, 9 aa, Human immunodeficiency virus 1
GPGHKARVL

SEQ ID NO: 15 - protein, 11 aa, Human immunodeficiency virus 1
KAFSPEVIPMF

SEQ ID NO: 16 - protein, 9 aa, Human immunodeficiency virus 1
VLAEAMSQV

SEQ ID NO: 17 - protein, 9 aa, Human immunodeficiency virus 1
SLFNTVATL

SEQ ID NO: 18 - protein, 10 aa, Human immunodeficiency virus 1
RYPLTFGWCF

SEQ ID NO: 19 - protein, 9 aa, Human immunodeficiency virus 1
AVDLSHFLK

SEQ ID NO: 20 - protein, 10 aa, Human immunodeficiency virus 1
QVPLRPMTYK

SEQ ID NO: 21 - protein, 8 aa, Human immunodeficiency virus 1
RYPLTFGW

SEQ ID NO: 22 - protein, 10 aa, Human immunodeficiency virus 1
RFPLTFGWCF

SEQ ID NO: 23 - protein, 10 aa, Human immunodeficiency virus 1
RGPGRAFVTI

SEQ ID NO: 24 - protein, 9 aa, Human immunodeficiency virus 1
KLTPLCVTL

SEQ ID NO: 25 - protein, 15 aa, Human immunodeficiency virus 1
RIQRGPGRAFVTIGK

SEQ ID NO: 26 - protein, 9 aa, Human immunodeficiency virus 1
RAIEAQQHL

SEQ ID NO: 27 - protein, 15 aa, Human immunodeficiency virus 1
RIQRGPGRAFVTIGK

SEQ ID NO: 28 - protein, 10 aa, Human immunodeficiency virus 1
TVYYGVPVWK

SEQ ID NO: 29 - protein, 11 aa, Human immunodeficiency virus 1
RLRDLLLIVTR

SEQ ID NO: 30 - protein, 9 aa, Zaire ebolavirus
FLSFASLFL

SEQ ID NO: 31 - protein, 9 aa, Zaire ebolavirus
FQQTNAMVT

SEQ ID NO: 32 - protein, 9 aa, Zaire ebolavirus
KLTEAITAA

SEQ ID NO: 33 - protein, 9 aa, Zaire ebolavirus
RLMRTNFLI

SEQ ID NO: 34 - protein, 8 aa, Zaire ebolavirus
YQNNLEEI

SEQ ID NO: 35 - protein, 9 aa, Zaire ebolavirus
ATDVPSATK

SEQ ID NO: 36 - protein, 9 aa, Zaire ebolavirus
RLASTVIYR

SEQ ID NO: 37 - protein, 9 aa, Zaire ebolavirus
TTIGEWAFW

SEQ ID NO: 38 - protein, 9 aa, Zaire ebolavirus
GFRSGVPPK

SEQ ID NO: 39 - protein, 9 aa, Zaire ebolavirus
NQDGLICGL

SEQ ID NO: 40 - protein, 9 aa, Zika virus
IGVSNRDFV

SEQ ID NO: 41 - protein, 20 aa, Zika virus
IRCIGVSNRDFVEGMSGGTW

SEQ ID NO: 42 - protein, 20 aa, Zika virus
QPENLEYRIMLSVHGSQHSG

SEQ ID NO: 43 - protein, 20 aa, Zika virus
KGVSYSLCTAAFTFTKIPAE

SEQ ID NO: 44 - protein, 20 aa, Zika virus
FEATVRGAKRMAVLGDTAWD

SEQ ID NO: 45 - protein, 20 aa, Zika virus
HRSGSTIGKAFEATVRGAKR

SEQ ID NO: 46 - protein, 17 aa, Influenza A virus
ELLVLLENERTLDYHDS

SEQ ID NO: 47 - protein, 17 aa, Influenza A virus
ELLVLLENERTLDYHDS

SEQ ID NO: 48 - protein, 20 aa, Influenza A virus
TYVLSIIPSGPLKAEIAQRL

SEQ ID NO: 49 - protein, 11 aa, Influenza A virus
LYKKLKREITF

SEQ ID NO: 50 - protein, 16 aa, Influenza A virus
GFEMIWDPNGWTGTDN

SEQ ID NO: 51 - protein, 17 aa, Influenza A virus
GQASYKIFRIEKGKIVK

SEQ ID NO: 52 - protein, 17 aa, Influenza A virus
GWAIYSKDNSVRIGSKG

SEQ ID NO: 53 - DNA, 3822 bp, synthetic construct
  source 1..3822: mol_type = other DNA
atggcattcgtgtttctggtgctgctgcctctggtgtccagccagtgcgtgaacttcaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgccaatcctgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcggggactgcctcagggcttttctgctctggaacccctggtggatctgcccatcggcatcaacatcacccggtttcagaccctgcaccggtcctatctgacacccggcgattcttctagcggatggacagctggcgccgctgcctactatgtgggatacctgcagcctcggaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagaccggcaatatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgcaatggcgtgaagggctttaactgctacttcccactgcagtcctacggcttccagccaacatacggcgtgggctaccagccttacagagtggtggtgctgagcttcgagctgctgcatgctcctgccacagtgtgcggccctaagaaaagcaccaatctcgtgaagaacaaatgcgtcaacttcaatttcaacggcctgaccggcaccggcgtgctgacagagagcaacaagaagttcctgccattccagcagttcggccgggacattgccgataccacagatgctgtcagagatccccagacactggaaatcctggacatcaccccatgcagcttcggcggagtgtctgtgatcacccctggcaccaacaccagcaatcaggtggcagtgctgtaccagggcgtcaactgtacagaggtgccagtggccattcacgccgatcagctgacccctacttggcgggtgtactccacaggcagcaatgtgttccagaccagagccggctgtctgatcggagccgagcacgtgaacaatagctacgagtgcgacatccccatcggcgctggcatctgtgccagctaccagacacagacaaacagccccggcggcagcggatctgtggccagccagagcatcattgcctacacaatgtctctgggcgtcgagaacagcgtggcctactccaacaactctatcgctatccccaccaatttcaccatcagcgtgaccacagagatcctgcctgtgtccatgaccaagaccagcgtggactgcaccatgtacatctgcggcgatagcaccgagtgctccaacctgctgctgcagtacggcagcttctgcacccagctgaatagagccctgaccggaatcgccgtggaacaggacaagaacacccaagaggtgttcgcccaagtgaagcagatctacaagacccctcctatcaaggacttcggcggcttcaacttcagccagattctgcccgatcctagcaagcccagcaagcggagcttcatcgaggacctgctgttcaacaaagtgacactggccgacgccggcttcatcaagcagtatggcgattgtctgggcgacattgcagcccgggatctgatttgcgcccagaagtttaacggactgaccgtgctgcctcctctgctgaccgatgagatgatcgcccagtacacatctgccctgctggccggcacaatcacaagcggctggacatttggagctggcgctgccctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagccagcgctctgggaaaactgcaggacgtggtcaaccagaacgcccaggctctgaataccctggtcaagcagctgtcctccaacttcggcgccatcagctctgtgctgaacgatatcctgagcagactggaccctcctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaacttcacaacagcccctgccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccttggtacatctggctgggctttatcgccggactgattgccatcgtgatggtcacaatcatgctgtgctgtatgaccagctgctgtagctgcctgaagggctgttgcagctgtggctcctgctgcaagttcgacgaggacgacagtgagccggtgcttaagggcgtaaaacttcattacacttccggatga

SEQ ID NO: 54 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 55 - protein, 433 aa, Homo sapiens
MGRVQLFEISLSHGRVVYSPGEPLAGTVRVRLGAPLPFRAIRVTCIGSCGVSNKANDTAWVVEEGYFNSSLSLADKGSLPAGEHSFPFQFLLPATAPTSFEGPFGKIVHQVRAAIHTPRFSKDHKCSLVFYILSPLNLNSIPDIEQPNVASATKKFSYKLVKTGSVVLTASTDLRGYVVGQALQLHADVENQSGKDTSPVVASLLQKVSYKAKRWIHDVRTIAEVEGAGVKAWRRAQWHEQILVPALPQSALPGCSLIHIDYYLQVSLKAPEATVTLPVFIGNIAVNHAPVSPRPGLGLPPGAPPLVVPSAPPQEEAEAEAAAGGPHFLDPVFLSTKSHSQRQPLLATLSSVPGAPEPCPQDGSPASHPLHPPLCISTGATVPYFAEGSGGPVPTTSTLILPPEYSSWGYPYEAPPSYEQSCGGVEPSLTPES

SEQ ID NO: 56 - DNA, 9675 bp, Homo sapiens
  source 1..9675: mol_type = genomic DNA
gtcgctgcgcggctggccggtgaggccgcggcatggggcgagtgcagctcttcgagatcagcctgagccacggccgcgtcgtctacagccccggggagccgttggctgggaccgtgcgcgtgcgcctgggggcaccgctgccgttccgaggtgggcgcgggtcctcggggagggcctttggccgcacctgcgcggggccagggctcccggaagcggctgccgtcgcctggccccgggttgggcccgcggagtcgctgtcttcgccgcccgggactgctgggaagcgggctcggcggcgccggggaggtccgggcggcgccgcccttccgcgtctgcccttcctgccctctcgccgaggggcggcggggtcccggcggggcctgaagcgggtccactgtggaggacgccctgagcgcgggcgcacgcaggcctgcgaccctccccgtccgcgctcgcacctgctgggctccttcccgcaccctggctcccggcgcaggcgctcccgctgcagagccgctcctggcgcgggcgtgcggtcccggcggggtgcagacacctgctgcggctccctgcgccccctgccacccgccagcaggcctcgccggccccccgccgaggtggacgcctgggcctcggggactgggtatcgggttgctgcagtggccgctggcagggcgccctggaggtgagggccggcccgcgaggtgaggctgcctctgcccggcccttggctgtgagtgccgcgtccctgccggggacttctctaccttggcctcctgtgggaaccacctgggctgcttggcctgtgccacagcccctggacttgccgctggaccttgaccgctgggcccgcgcgtgggtaacagcaccgattttgtgcctgctgagaacccacagtctctgttctgtcctctcagcctcctgggacaggtgatggtgctgtggccctgaccctgcaccgcctgaggctgtggggcctcggaagaagccgggtggtaggtgcttctgtcaggctcctggccccactgggcctcctgggatgaccccacagcctcccttcctctgtcccaaccttgagtttggggtctgggtgggggcaggtgtggccgtggcagggcagagatagcacacacatctcttgggtacaggtgtggtgtgtggtgaggccggctggatcctggttgagggctgtgcaggaccctgtcgtccttcccctggcgtggcctggggtgagcaggccattgcttaccacagggaagggacggccccgcggtgcgtgactggcctgggtgtgcaggctcccctggcctccgggtggacgtggtgggtgggttgaatgctcacgcctgtaatcccagcactttgggcagctggcccaagagccgagggagcactggctgatggtcctacctcacagaggctggtctgaatgagggagaaaaaggagggtcgtgagctaaggagtaggagatgggccctgatgggccagagagcacatgattcaggctgctgcaagtgaggtgggagggtgtaccggggcccccgggggtttcccccagccctgcctgggatgggaacagaaggaccatggtttcttttcctcattcagaggatgcttgtcaatcctgctaggagccagtagggaggccctcagcccccaggaggacagcagccctgcctgggccccagagcttgctcacttactctgccgatgagtgtcggtgacagtcagcacccagccgtgaggacaccggctgtctaaagggctgacgcggagggaggcctgggagggagcagttttctggagctcaggctggcccagtccagggctggggttcaggcaggaccacttcttgttctgctgttactgggatgggcgaccagggccgacagtggttctcggagcactcatggtgggcccgatgcaattctctccacgtgccacgggccagcctcagttaaccctcagcgccctggcaagtggcacggttgttactctcatgatacagatttggaaacaggactgggggagtggcttacaggtcacagcttctgtggcaggcgtgagtcctttgggatgaggatgaggagtgggtgggtcttcatggtccaagtgcaggtggtgcaagggaggggctgctgaacaggtgggagccccagctggagaagaaagttccagtggtcacttgagaacgcagcccagtcatgatcttccttagagtcagagaggcctgcagcctccagcagcttgtccttgcttgtgcatctgaacacaggaaacattttttatttttatttttttaatttattttttcgagacggagtctccctctgtcgcccaggctggagtgtgccggcgcgatctcggctcactgcaagctccgcctccccggttcacgccattctcctgcctcagcctccttaagagctgggactacaggcgcccgccaccacacccggctaatttgtgtatttttagtagagacggggtttcaccgtgttagccaggagggtctggatctcctgacctcgtgatccgcccgcctcggcctcccaaagtgctgggattacaggcgtgagccaccgtgcctggccaggaaacattttttaaacctgcatgggggaggagggacagggcttgcagggcccctgcaaagaggtgcaacgttcaagtccagaaggcggaagaagtcacactctgactaggggactgccactgagccactcagtcctgcagccaagccctgcctggtggctttcctgttgcgaaaggctcggttcccttcagcgtgttctgccatcacaagcagtctgcaagctggaaagaagagggagcggggtcgtatgccgccagcagcagggagacccctcacaatggaccccatgtattgctctggcttctccgtgggcagaactgcatcctgtggtcctgcctgattcgatgatttgaaaaggcagaaacatggccgctgccattggcctggccagtgaaaacgggctccctgggaaattcacccagcctgacctgaagcagttgctgagaggcagaggagcagctccaggtgttttggcagggaagcagctggcagctgtgcgggagccccttgcctcccgtgttcttactgaggagtgtgtggggcagctcgggacaggtttcactttggacagtggttggaggctgcttactctggcagagtcatctcaaagagccttggtgtatgtgcaaaacggggccatctgcatgggcgttgggaagatcacagaacctggacccttaggatgacccaagggtcagttgcatactgtttgtttttgtggagtcaatgtcccatgacggttttcgcgatgctttgctagtaggagcagtggcgtgtgaggctcccggttaaataccatgagattctaagcggaattctgggtctcctggtatgcacaggtaaaattacagtagacctgtcccactctttgccagaacctcgtacttgtctgcagtttgactggaataaggggtgtttgtgttccacctccctccaacgtgtgttgagacatttatttatttatttatttatttacgttgctcttgttacccaggctggagtgcagtggtgcaatctcagctgctcaccgcaacctctgcctccctggttcaagtgattcttctgcctcagactcccgagtagctgggactacaggcacgcgccaccaggcctggctaattttgtatttttagtagagacggggtttctccgtgttggtcaggcgggcctggaactccgaacctcaggtgatccgcccgcctcagcctcttaaagtgctgggattacaggcgtgagccactgtgcctggccccaagacattgattttatttgagatgaggtctcactttgtcacccagactggagtgcagtggtacaatcttggctcactgcaactccttcctccggggctcccaccacagcctcctgagtgactgggaccaggggcacgcgccaccacacccagctaatctttggtagttttggtagagatggggtttcaccgtgttacctaggctgctgtcacacttctggcctcaagcaatccgctcgccttggccccccaaagtgttgggattacaggcgtgagccactgcgcccagctgtgtcgagacatttaatatttgtcaatctgatggattgaaatggtaggtcatttgggctttccctgcaagctccaccttccgggttcacgccattctcctgcctcagcctcctgagtagctgggactacaggcgcccgccaccacacccggctaattttttgtatttttagtagagacgggggtttcaccgtgtcagccaggatgatctcgatctcctgacctcgtgatctgcccgcctcggcctcccacagtgctgggattacaggctgagccactgcgcccggccgggttttctgtaatttctagtgaggttggacatctgttccagttacctactgctacgtaaaagtcgcggtcacctcaagtcagacaccattcttaaaacagttaacacaaccattttattatatctcacagcgttgtgggttaggaatctggacagtgagggcagggttaatcttcagttctgtgccaccagctgggatcacttgagggtgcttggctggcatacgggttagtctggggaccactgccccctttccggcatcctctgtgggttccagcatttttcttttttttttctttttgagatgaagtctcgctctgtcacccaggctaaagtgcatttgtgcgatctcagctcactgcaacctctgcctcctgggtttaagtgatcctcatgcctcagcctcccaagtagctgggattacaggcgtgtgccaccatgcctggctaatttttgtattttttttttgagatggagtttcactcttgttgctcaggctggagtgcagtggcgcgatctcggctcactgcaacctccgcctcccaggttcaagcgattctcctgcctcagcctccctggtagctgggattacaggcatgtgccaccatgcccagctaatttcgtatttctagtagagatggggtttctccatgtcggtcaggctggtctggaactcctgacctcaggtgatctgcctgccttggcctcccgaagtgctgggattacaggcatgagccaccgtgcccggccttttttgtatttttagtagagatggggtttcaccatgtcagccaggctggtcttgaactcctgacctcaagtgatccacccgccttggcctcccacagtgctgggattacaggtgtgagccactgcacccagctgggtcccagcatttgcgggattcaaggtggggatgtgcagtctgtcaggagtgttaaaagcacggcagcatttccagtccccaggcctcctctccttgcttcagccacgcccaccctcctctgtgcctgctcatgcccatgtgcttctgttgggctgtctgctggtccacaggaggattactggtgttccggattttcagcatgaacacagtatggggtctggcccgaggagggcctggctaacagagcatggattcaaaatcagagcagagctgggtaccgtggctcatgcctgtaatcccaacactttgggaggctgaggcaggtggattgcttgagcttaggagtttgagaccagcctggacagcatggcaaaaccctgtctctataagaaaaacaaaaaacaaaaaacaaaacccaggtgtggtggcacgtgcctgtagtcctagctacttgcggggctgagatgggtggatcgcttgaacacaggaggttaaggctgcagtgagccgagatcgtgccactgcacactagcctgggcggcgggagtgagaccgtgtctcaaaaacaacaacaacaacaacaacaacaaaaaaacaaaaaatggaccgaggttggggtggcaaggctgggctggaatggaggctgaggacaggccctggcagcgcccgggggtagagggcgtgcagcaggaagcaagggccaccctcagggatgggggactgggcctggcagctgccgaggggtaggcaggatgcatgtggagagccacgtggggctgcggctgagccaaggattgtgagaggaatcctgctgctctgggtggcaggcagaaggcctgggccacaggtggggcctgctggggagaccacaggtgaggcaggtatggctgaccctgggcgctcggcacaccggtactcctcctgcctggctgctccctcagcaggtgtcagagtctgagtcaccttttcctcacgtgacccccaccaggaccaggcagggaggtggccatctttggccctgactcagccctgcagggagaagaggggctggaggtgcaggtggcctgccactgtctcccccaacccctgcccacctgggtcttgtgctgtgtgtacttctgctgcgtctgtcctcccttgtgaccagcttctgctggacctgattgtccagccatgatcttctgatcatcatggtttctggacacacatctgttggactaaggaatttgtgttcctggccacagaacagtgccacctctgcctcaagcagttcctggtctgggcactcccccagggaggccggagaggactgagccgtctcctgatgcagaaagtcagccccgctgggcagtgctcgcccaggaagtagggccccacccaacggtgtgctgactgtggaacactgtgtctgcctaggcgggtctggccacattcctgcagcatgatggttggcagttagcttcctggagtgtggacgggtccttccctgcctgactgacttgtccacagggcagcaagaagatgcgccccttggtgtgtcccgagtgtctcctgtaggcagctgagctttcgcaggtctggctgggagccaaggttaccaatctggggggtaacccagccatcatctggatgagcgttggggtctttctgctctggattgcagggcagacgtcgttttgtgctggtcaggcccctcgcctgcctgattcccttggagccccccaagacaggagatgggcagatgtcctctgaagtggcatgggctgcttgctcactggccttcccaggaccctgcccaggtggtgctgcttggccccaggtcgaattcctgtgctggcagcaagcaggggcctgggccgtcggctggctggggctcatgcagccatcccctttgcagccatccgggtgacctgcataggttcctgcggggtctccaacaaggctaatgacacagcgtgggtagtggaggagggttacttcaacagttccctgtcgctggcagacaagggtaagtttgggagccagttcccgagtggtgacccctgggggcagcccttggagtggggcctgtctgtcctgtcctccccctttcctagatctgcctcttggatctggcactggcccatcttgtcccagggttgtgggccctgatggctttctcataggcgctgggtggctgaaggggccacctcccccttgtgctggatttgggtgggctctgacccctggcacagctgtttcacccttgccccattcccaaaccactctcctgttcaagccaagctcttatcctcagtccttcacccaagccacagccctgtgctgctgtggcgctggtgactgagacagggagacccagtgtgagctggtcagctgcagtgcccactcttatcctggccctgtggccacctcaggccacctcagggggggactgccccccacctccctcctgtctctgcagggagcctgcccgctggagagcacagcttccccttccagttcctgcttcctggtgagagcccagcctggacaggcctggtgatgaccaactggtcctgggaggtgggctctgcagggtggaaggcagccaggtaccagtgcctgctctctccccagccactgcacccacgtcctttgagggtcctttcgggaagatcgtgcaccaggtgagggccgccatccacacgccacggttttccaaggatcacaagtgcagcctcgtgttctatatcttgagccccttgaacctgaacagcatcccagacattgaggtgaggatggcacagtgacctccttggtgggtccccaccctcatggaggctggggaagagctgggcaggcactgcttcatcccagcgtcctgtccccagctgaggtgaggggggccagggtctggaggcagtggcctctttgccctgagctgactgctgatgtccaggggcagggcgtggcagggctgaggggcctgtcccaatgccacagggatccagcctgcgcacacctgctaagcccctccctggccctccccccaagcaacccaacgtggcctctgccaccaagaagttctcctacaagctggtgaagacgggcagcgtggtcctcacagccagcactgatctccgcggctatgtggtggggcaggcactgcagctgcatgccgacgttgagaaccagtcaggcaaggacaccagccctgtggtggccagtctgctgcaggtcagagcccccgccagttgcctggaccggcctcctggggtgggctggcagcctgcgcaggctcacaggctggtccttccccagaaagtgtcctataaggccaagcgctggatccacgacgtacggaccattgcggaggtggagggtgcgggcgtcaaggcctggcggcgggcgcagtggcacgagcagatcctggtgcctgccttgccccagtcggccctgccgggctgcagcctcatccacatcgactactacttacaggtttggtgctgctgggggctgggtggtctgaggcctagtggccttggccctggcccctcatgccccacctcaatcctgtccaggtctctctgaaggcgccggaagctactgtgaccctcccggtcttcattggcaatattgctgtgaaccatgccccagtgagcccccggccaggcctggggctgcctcctggggccccacccctggtggtgccttccgcaccaccccaggaggaggctgaggctgaggctgcggctggcggcccccacttcttggaccccgtcttcctctccaccaagagccattcgcagcggcagcccctgctggccaccttgagttctgtgcctggtgcgccggagccctgccctcaggatggcagccctgcctcacacccgctgcaccctcccttgtgcatttcaacaggtgccactgtcccctactttgcagagggctccggggggccagtgcccactaccagcaccttgattcttcctccagagtacagttcttggggctacccctatggtgagtcgacagccagggcttggcagggaggggacgccaagagccccacgcagaccctgctttcttcccgcagaggccccaccgtcttatgagcagagctgcggcggcgtggaacccagcctgacccctgagagctgaccccgtgctgccttctccaggcaggcctggcctctgccctgggactggggcgcccagggcctcgtgccttctctcttggcctagcctggcccactcaggacctgcccagcctctgccagctcctctggcatccgccctcttctccctggggctggggtgggggtggcagggagctgggacctggagagacaactcctgtaaataaaacactttatttgtaga

SEQ ID NO: 57 - DNA, 228 bp, synthetic construct
  source 1..228: mol_type = other DNA
cgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 58 - DNA, 2950 bp, synthetic construct
  source 1..2950: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatcgattgactagtgccaccatggggcgagtgcagctcttcgagatcagcctgagccacggccgcgtcgtctacagccccggggagccgttggctgggaccgtgcgcgtgcgcctgggggcaccgctgccgttccgagccatccgggtgacctgcataggttcctgcggggtctccaacaaggctaatgacacagcgtgggtagtggaggagggttacttcaacagttccctgtcgctggcagacaaggggagcctgcccgctggagagcacagcttccccttccagttcctgcttcctgccactgcacccacgtcctttgagggtcctttcgggaagatcgtgcaccaggtgagggccgccatccacacgccacggttttccaaggatcacaagtgcagcctcgtgttctatatcttgagccccttgaacctgaacagcatcccagacattgagcaacccaacgtggcctctgccaccaagaagttctcctacaagctggtgaagacgggcagcgtggtcctcacagccagcactgatctccgcggctatgtggtggggcaggcactgcagctgcatgccgacgttgagaaccagtcaggcaaggacaccagccctgtggtggccagtctgctgcagaaagtgtcctataaggccaagcgctggatccacgacgtacggaccattgcggaggtggagggtgcgggcgtcaaggcctggcggcgggcgcagtggcacgagcagatcctggtgcctgccttgccccagtcggccctgccgggctgcagcctcatccacatcgactactacttacaggtctctctgaaggcgccggaagctactgtgaccctcccggtcttcattggcaatattgctgtgaaccatgccccagtgagcccccggccaggcctggggctgcctcctggggccccacccctggtggtgccttccgcaccaccccaggaggaggctgaggctgaggctgcggctggcggcccccacttcttggaccccgtcttcctctccaccaagagccattcgcagcggcagcccctgctggccaccttgagttctgtgcctggtgcgccggagccctgccctcaggatggcagccctgcctcacacccgctgcaccctcccttgtgcatttcaacaggtgccactgtcccctactttgcagagggctccggggggccagtgcccactaccagcaccttgattcttcctccagagtacagttcttggggctacccctatgaggccccaccgtcttatgagcagagctgcggcggcgtggaacccagcctgacccctgagagcggccgcaaaaaacgccgccagcgccgccgcgcgcatcagaacagccagacccattgagcggccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 59 - DNA, 2977 bp, synthetic construct
  source 1..2977: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatcgattgactagtgccaccatggggcgagtgcagctcttcgagatcagcctgagccacggccgcgtcgtctacagccccggggagccgttggctgggaccgtgcgcgtgcgcctgggggcaccgctgccgttccgagccatccgggtgacctgcataggttcctgcggggtctccaacaaggctaatgacacagcgtgggtagtggaggagggttacttcaacagttccctgtcgctggcagacaaggggagcctgcccgctggagagcacagcttccccttccagttcctgcttcctgccactgcacccacgtcctttgagggtcctttcgggaagatcgtgcaccaggtgagggccgccatccacacgccacggttttccaaggatcacaagtgcagcctcgtgttctatatcttgagccccttgaacctgaacagcatcccagacattgagcaacccaacgtggcctctgccaccaagaagttctcctacaagctggtgaagacgggcagcgtggtcctcacagccagcactgatctccgcggctatgtggtggggcaggcactgcagctgcatgccgacgttgagaaccagtcaggcaaggacaccagccctgtggtggccagtctgctgcagaaagtgtcctataaggccaagcgctggatccacgacgtacggaccattgcggaggtggagggtgcgggcgtcaaggcctggcggcgggcgcagtggcacgagcagatcctggtgcctgccttgccccagtcggccctgccgggctgcagcctcatccacatcgactactacttacaggtctctctgaaggcgccggaagctactgtgaccctcccggtcttcattggcaatattgctgtgaaccatgccccagtgagcccccggccaggcctggggctgcctcctggggccccacccctggtggtgccttccgcaccaccccaggaggaggctgaggctgaggctgcggctggcggcccccacttcttggaccccgtcttcctctccaccaagagccattcgcagcggcagcccctgctggccaccttgagttctgtgcctggtgcgccggagccctgccctcaggatggcagccctgcctcacacccgctgcaccctcccttgtgcatttcaacaggtgccactgtcccctactttgcagagggctccggggggccagtgcccactaccagcaccttgattcttcctccagagtacagttcttggggctacccctatggtgagtcgacagccagggcttggcagggaggggacgccaagagccccacgcagaccctgctttcttcccgcagaggccccaccgtcttaggccgcaaaaaacgccgccagcgccgccgcgcgcatcagaacagccagacccattgagcggccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 60 - DNA, 58 bp, synthetic construct
  source 1..58: mol_type = other DNA
ggtctctctggttagaccagatctgagcctgggagctctctggctaactagggaaccc

SEQ ID NO: 61 - DNA, 5461 bp, synthetic construct
  source 1..5461: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatcgattgactagtgggtctctctggttagaccagatctgagcctgggagctctctggctaactagggaacccgccaccatggccttcgttttcctggtgctgctgcccctggtgagcagccagtgcgtgaatctgaccaccagaacccagctgcctccagcctacacgaactccttcacacgtggagtgtattaccctgataaggtgttcagaagctccgtgctgcattctacacaagatctgttcctacctttcttcagcaacgtgacctggttccacgccatccacgtgagcggaacaaacggcaccaagcggttcgacaatcctgtgctgcccttcaacgatggcgtgtactttgccagcaccgagaagagcaacatcatccggggctggatcttcgggacaacactggactctaaaacccagagcctgctgatcgttaataacgctacaaatgtggtgattaaggtttgtgaattccagttttgcaacgacccattcctgggagtgtactatcataagaacaacaagagctggatggaaagcgaatttagagtgtactcctccgcgaacaattgcaccttcgagtacgtgagccaacctttcctgatggacctggaaggcaagcagggtaatttcaaaaacctgagagagttcgtcttcaagaacatcgacggctacttcaagatctacagcaagcacacccccatcaacctggtgcgggatctgcctcagggcttcagcgccctcgagcccctagtcgacctgcctatcggcatcaacatcaccagattccaaaccctgcttgccctgcacagatcttacctgaccccaggcgacagcagcagcggctggaccgccggggcagccgcctattacgtgggatacctgcagcctagaacattcctgctgaaatacaatgagaacggtacgatcacagacgcagttgattgcgccctggaccccctgagcgagacaaagtgcaccctgaaaagcttcacagtggaaaagggcatctaccagaccagcaacttccgggtgcagcccaccgaatccatcgtgcggtttcctaacatcaccaacctgtgtccttttggcgaggtgtttaacgccacacggttcgctagcgtgtacgcctggaacagaaagcggatttctaactgcgtggccgactacagtgttctgtacaactctgctagtttcagtaccttcaagtgttacggcgtctctcctactaagctgaacgacctgtgcttcaccaacgtgtacgccgactcgttcgtgatcaggggcgatgaggtgcggcagatcgcccctggccagacaggaaagatcgccgactacaactacaagctgcctgacgattttaccggctgcgtgatcgcctggaactctaacaacctggacagcaaggtgggcggcaattataattacctgtaccgcctctttagaaagtctaatctgaagcccttcgaaagagatattagcaccgagatctaccaagccggcagcacaccttgcaacggcgtcgagggcttcaactgctacttcccactgcaatcttacggcttccagcctacaaacggcgtgggctaccagccttacagagtggtggtgctgagctttgaactgctgcacgcccctgcgaccgtgtgtggacctaagaagagcacaaacctggtcaagaacaagtgcgtgaatttcaattttaatggcctgaccgggaccggagttctgaccgagagcaacaaaaaattcctgccctttcagcagttcggcagagacatcgcagacaccacagacgccgtgagagatcctcagaccctggagatcctggacatcaccccgtgctctttcggaggcgtcagcgtgatcacccctggtaccaacaccagcaaccaggtggccgtgctgtaccaggacgtcaattgcacagaagttcctgttgccatccacgccgatcagctcacccccacctggcgggtgtatagcaccggtagcaatgtgttccagacaagagccgggtgcctgatcggagccgaacacgtgaacaatagctatgaatgtgatatccccatcggcgctggcatctgtgccagctaccagacccagacaaatagccccggctccgcttcttctgtggccagccagagcattatcgcctacacaatgagcctgggcgctgagaactctgtggcctacagcaacaacagcatcgccattccaaccaacttcaccatcagcgtgaccaccgagatcctgcctgtgagcatgaccaagaccagcgtggactgcaccatgtacatctgcggagactctaccgagtgcagcaacctcctgctgcaatacggcagtttttgcacgcagctgaacagagccctgacaggcatcgccgtggaacaggataagaacacacaggaggtgttcgcccaggtgaaacagatctacaagacacctcctatcaaagacttcggcggcttcaacttttcccagatcctgcctgatcccagcaagcctagcaagagaagcttcatcgaggacctgctgttcaacaaagtgacactggccgacgctggcttcatcaagcagtacggcgactgtctgggagacatcgccgccagagatctgatctgcgcccagaagttcaacggactgaccgtgctgcctccactgctaactgatgagatgatcgcccagtacaccagcgctctgctggctggcaccatcacatcaggctggaccttcggcgctggcgccgcactgcagatccccttcgccatgcagatggcctatcggttcaatggcatcggcgtcacccaaaacgtcctgtatgaaaaccaaaaactgattgctaatcagttcaactctgccatcggcaaaatccaggatagcctgtccagcaccgcctctgctctgggcaagctgcaggacgtggtgaaccagaacgcccaggctctgaataccctcgtgaagcaactttcctctaacttcggggccatctctagcgtgctgaacgacatcctgagcagactggaccctcctgaggcggaggtacagatcgatcggctgatcaccggcagactgcagtcacttcagacttacgtgacacagcagctgatccgggccgccgaaatcagggccagcgccaacctggcagctacaaagatgagcgaatgcgtgctgggacagtccaagagagtggatttctgtggcaagggctaccacctgatgagcttcccccagtccgccccccacggcgtggtcttcctgcacgtgacatatgtgcccgcccaggagaagaattttactaccgcccctgccatctgccacgatggcaaggctcacttccctagagagggcgttttcgtgtctaacggcacccactggttcgtgacccaaagaaatttctacgagcctcagattatcacaaccgataacactttcgtgagcggcaactgcgacgtggtgatcggcatcgtgaacaacaccgtctacgatcccctgcagcccgagctggactcattcaaggaagaactggataagtactttaaaaaccacacaagtccagacgtggacctgggcgacatctctggcattaacgcctctgtggtgaacatccagaaagagatcgacagactgaacgaggtggccaagaacctgaacgagtccctcatcgacctgcaggagctgggcaagtacgaacagtacatcaagtggccttggtacatctggctgggcttcatcgccggcctgatcgccatcgtgatggtgaccatcatgctgtgctgcatgacaagctgctgtagctgcctgaagggttgttgtagctgcggcagctgctgtaaattcgacgaggatgacagcgagcctgtgctgaagggcgtaagcggccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 62 - DNA, 2373 bp, synthetic construct
  source 1..2373: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatcgattgactagtgggtctctctggttagaccagatctgagcctgggagctctctggctaactagggaacccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 63 - protein, 9 aa, Human immunodeficiency virus 1
SLYNTVATL

SEQ ID NO: 64 - protein, 1276 aa, synthetic construct
MAFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTSG

SEQ ID NO: 65 - protein, 4 aa, synthetic construct
RRAR

SEQ ID NO: 66 - protein, 4 aa, synthetic construct
GSAS

SEQ ID NO: 67 - RNA, 57 nt, synthetic construct
  source 1..57: mol_type = other RNA
ggtctctctggttagaccagatctgagcctgggagctctctggctaactagggaacc

SEQ ID NO: 68 - protein, 4 aa, synthetic construct
PSAP

(19) United States
(12) Patent Application Publication
(10) Pub. No.: US 2025/0000969 A1
(43) Pub. Date: Jan. 2, 2025

(54) ANTIGEN DELIVERY PLATFORM AND METHODS OF USE

(71) Applicant: Intima Bioscience, Inc., New York, NY (US)

(72) Inventors: Modassir Choudhry, New York, NY (US); Thomas Henley, Soham (GB)

(21) Appl. No.: 18/757,948

(22) Filed: Jun. 28, 2024

Related U.S. Application Data
(63) Continuation of application No. PCT/US22/82512, filed on Dec. 29, 2022.
(60) Provisional application No. 63/294,639, filed on Dec. 29, 2021.
(60) Provisional application No. 63/339,225, filed on May 6, 2022.

(51) Int. Cl. A61K 39/215; A61K 47/69; A61P 37/04; C07K 14/005; C12N 5/074; C12N 9/22; C12N 15/11; C12N 15/90

(52) U.S. Cl. CPC A61K 39/215; A61K 47/6901; A61P 37/04; C07K 14/005; C12N 5/0696; C12N 9/22; C12N 15/11; C12N 15/907; C12N 2770/20022; C12N 2770/20034; C12N 2770/20052; C12N 2770/20071

(57) ABSTRACT

Provided herein are compositions and methods for inducing an immune response in a subject, wherein the compositions comprise apoptotic cells, extracellular vesicles (EVs), and combinations thereof. Various compositions comprising mRNA encoding a microbial antigen are also provided. Further provided herein are compositions engineered to overexpress the human arrestin domain containing protein 1 [ARRDC1].

### CROSS-REFERENCE TO RELATED APPLICATIONS

This application is a continuation of and claims priority to, and the benefit of, International Application No. PCT/US2022/082512, filed Dec. 29, 2022 which claims priority to, and the benefit of, U.S. Provisional Patent Application Ser. No. 63/294,639, filed Dec. 29, 2021, and U.S. Provisional Patent Application Ser. No. 63/339,225, filed May 6, 2022, the contents of which are incorporated by reference in their entirety and commonly owned.

### SEQUENCE LISTING

This application contains a Sequence Listing, which has been submitted electronically in ST.26 format and is hereby incorporated by reference in its entirety. Said copy, created on Jun. 28, 2024, is named 199827-763301—SL.xml and is 95,953 bytes in size.

### BACKGROUND

Current vaccine development strategies predominantly rely on the delivery of live-attenuated or inactivated forms of microbial pathogens in combination with immune stimulating adjuvants in order to elicit a host immune response and induce the production of lasting antigen-specific antibodies and memory lymphocytes. However, these strategies rely on non-physiological methods of antigen exposure and non-physiological adjuvants, which may not sufficiently generate the immune response desired for a vaccine.

Current mRNA, protein, and viral vector-based vaccines have certain limitations, such as their requirement for excipient adjuvants to activate the recipient immune system, or to deliver the viral antigenic payload. These include the artificial lipid nanoparticles delivering the mRNA, or MF59, AS03, Alum, ISCOMATRIX, and Matrix-M chemical emulsions for example, or the adenoviral protein antigens themselves that stimulate innate immune cells. Adjuvants are required to increase the effectiveness of vaccines and their use can cause side-effects including local reactions (redness, swelling, and pain at the injection site) and systemic reactions (fever, chills, and body aches). Further, there have been modest but real morbidity and rare mortality associated with Thrombosis with Thrombocytopenia Syndrome (TTS) and Myocarditis, secondary to the current COVID-19 vaccines.

The size constraint of the adenoviral vector genome, and the limited length of stable mRNA that can be produced and packaged into nanoparticles, restricts the number and size of nucleic acid-encoded antigens and epitopes that can be delivered in these vaccines. Thus, these vaccines are constrained in their ability to provide multiple immunodominant proteins to address emerging pandemic variants, or to easily combine multiple pathogens into one vaccine.

Therefore, novel vaccine strategies are needed that more closely mimic and modulate physiological immune responses.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### BRIEF SUMMARY

Provided herein are, inter alia, cellular vaccines, allogeneic universal vaccine generation cells and methods for generating and using the same. Also disclosed are poly-pathogenic polyvalent universal cellular vaccines that comprise multiple pathogenic viral, bacterial, protozoal, helminthic proteins and/or other antigenic determinants. Exemplary embodiments comprise a seasonal pan-respiratory virus vaccine that protects against multiple coronavirus variants, multiple influenza variants, and respiratory syncytial virus (RSV). Embodiments disclosed herein take advantage of the inherent and theoretical unlimited payload of a cellular vaccine used for antigen presentation and rationally designed immunization strategies.

Provided herein are universal vaccine cells (UVC) rationally designed to mimic the natural physiologic immunity engendered post the viral infection of host cells and resulting cell lysis. In one or more exemplary embodiments, induced pluripotent stem cells were genetically engineered, for instance by use of a CRISPR nuclease system, to delete MHC-I expression and simultaneously overexpress a NK Ligand (MICA) adjuvant to enhance rapid cellular apoptosis. The ensuing immune microenvironment naturally potentiates a hyper-immune response to the target viral antigen. By way of non-limiting example, cells were further engineered to express the original Wuhan or WA1/2020 SARS-CoV-2 spike viral antigen. These UVC were used to immunize non-human primates in a standard 2-dose, prime+boost vaccination resulting in robust neutralizing antibody responses. The antibody titers and resulting immunogenicity were on par with the neutralizing antibody titers demonstrating immune protection with the emergency use authorized and now commercially available mRNA vaccines (1e3 nAb titer). Animals vaccinated with WA1/2020 spike antigens were subsequently challenged with 1.0×105 TCID50 infectious B.1.617.2 (Delta) SARS-CoV-2 in a heterologous challenge, which resulted in an approximately 3-log order decrease in viral RNA load. This heterologous viral challenge reflects the ongoing real-world experience of WAT/2020 spike vaccinated populations exposed to rapidly emerging non-Wuhan variants like Delta and Omicron.

As set forth herein, some embodiments deliver multiple variant antigens (polyvalency) and can be rapidly manufactured at scale as soon as new viral variants are discovered. Further, embodiments wherein multiple antigens (polyvalency) from different pathogens (poly-pathogenicity) are incorporated are also disclosed herein.

Provided herein is a genetically engineered human cell comprising: (a) a genomic disruption in at least one human leukocyte antigen (HLA) gene or at least one transcriptional regulator of an HLA gene; and (b) an exogenous nucleic acid encoding a cell surface protein that binds to a protein expressed on the surface of a phagocytic or cytolytic immune cell, or a functional fragment or functional variant of the cell surface protein, wherein the binding results in the activation of phagocytic or cytolytic activity of the immune cell.

Provided herein is a population of genetically engineered human cells as disclosed herein.

Provided herein is a pharmaceutical composition comprising the genetically engineered human cells as disclosed herein, and an excipient. Some embodiments provide a unit dosage form comprising a composition or genetically engineered human cell as disclosed herein.

Provided herein is a genetically engineered human cell comprising: a genomic disruption in at least one human leukocyte antigen (HLA) gene or at least one transcriptional regulator of an HLA gene; and a genomic modification that results in overexpression of at least a portion of a human arrestin domain containing protein 1 [ARRDC1] or a functional variant thereof, wherein the overexpression is in an amount sufficient to result in increased vesicle formation as compared to a comparable cell that lacks the genomic modification.

Provided herein is a genetically engineered human cell comprising: a genomic modification that results in overexpression of at least a portion of a human arrestin domain containing protein 1 [ARRDC1] or a functional variant thereof, wherein the overexpression is in an amount sufficient to result in increased vesicle formation as compared to a comparable cell that lacks the genomic modification; and an exogenous nucleic acid encoding a cell surface protein, wherein the cell surface protein binds to a protein expressed on the surface of a phagocytic or cytolytic immune cell, or a functional fragment or functional variant of the cell surface protein, wherein the binding results in the activation of phagocytic or cytolytic activity of the immune cell.

Provided herein is a method of making a population of genetically engineered human stem cells, the method comprising: obtaining a population of human stem cells; inducing a genomic disruption in at least one HLA gene or at least one transcriptional regulator of the HLA gene; and introducing a nucleic acid encoding an exogenous cell surface protein that binds to a protein expressed on the surface of a phagocytic or cytolytic immune cell, or a functional fragment or functional variant of the exogenous cell surface protein, wherein the binding results in the activation of phagocytic or cytolytic activity of the immune cell; to thereby produce a population of genetically engineered stem cells.

Provided herein is a method of inducing an immune response to an antigen in a subject, the method comprising: administering to a subject the population of genetically engineered human cells provided herein, thereby inducing an immune response to an antigen.

Provided herein is a method of inducing an immune response to an antigen in a subject, the method comprising: administering to a subject the population of genetically engineered human cells provided herein, thereby inducing an immune response to an antigen.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the amino acid sequence SARS-CoV-2 Spike (S) protein, and individual domains therein (SEQ ID NO: 1).

FIG. 2 is a flow chart showing an exemplary workflow of the cellular vaccine platform described herein.

FIG. 3 is a representation of a vaccine cell described herein.

FIG. 4A is an exemplary illustration of an immune response to a viral infection. FIG. 4B shows an exemplary response resulting from vaccination using composition provided herein. A Universal Vaccine Cell (UVC) delivers an antigen-loaded living cell in-vivo, genetically engineered to elicit a natural physiologic, and potent activation of the immune system for production of neutralizing antibodies and lasting cellular immunity. The UVC can possess self-adjuvating properties via robust lysis by innate immune cells, activating the cellular and antibody immune response similar to a native host response to viral infection thus recapitulating natural physiologic immunity.

FIG. 5 shows exemplary inhibitory and activating receptors on NK cells and their cognate ligands on target cells. Any one of these receptors can be ectopically or endogenously expressed by a vaccine cell described herein.

FIG. 6 is an exemplary schematic showing that NK cells recognize platforms cells, for instance cells missing MHC-I components as either foreign, virally infected or pathogenic, and target them for cytolysis.

FIG. 7 shows that platform cells, for instance CRISPR Knockout B2M (a component of the MHC class I complex) iPSC cells demonstrate an abolished expression of MHC I even after IFN-gamma stimulation.

FIG. 8A shows that B2M deficient platform cells described herein fail to activate the proliferation of MHC-mismatched T cells compared to control iPS cells, demonstrating the potency of cells described herein. FIG. 8B shows a flow cytometry plot on Day 7 of platform cells differentiated into CD31+CD144+ endothelial cells.

FIG. 9 shows flow cytometry plots, acquired 48 hours post transfection, of platform cell-derived endothelial cells described herein, overexpressing NK-activating ligands of Table 6.

FIG. 10 shows that upon lysis of endothelial cells expressing variants of the SARS-CoV-2 spike protein (full length and spike S1 subunit), both protein antigen variants could be detected abundantly and showed a dose-dependent increase with vaccine cell number.

FIG. 11 is a schematic of the SARS-CoV-2 virus and spike protein structure.

FIG. 12 shows a natural killer (NK) cell killing assay. Shown is percent of dead target cells either K562 or iPSC-derived endothelial cells (differentiated from platform cells) at increasing effector-to-target (E:T) ratios.

FIG. 13 is a schematic of a Universal Vaccine Cell (UVC). The UVC comprises a deletion in the B2M locus (KO-B2M), rendering it MHC-I deficient. The UVC also comprises two knock-in (KI) constructs. One KI construct expresses a NK ligand MICA on the cell surface of the UVC, and another KI construct expresses SARS-CoV-2 spike protein and nucleocapsid phosphoprotein intracellularly.

FIG. 14A shows the full-length amino acid sequence of the SARS-CoV-2 nucleocapsid phosphoprotein (SEQ ID NO: 54).

FIG. 14B shows a schematic of the expression cassette of the SARS-CoV-2 Spike (SPIKE) protein and nucleocapsid phosphoprotein (N) in the UVC, connected by a T2A peptide cleavage sequence. The construct is driven by a EF1a promoter.

FIG. 14C shows that Nucleocapsid phosphoprotein has the highest density of epitopes across the SARS-CoV-2 genome. The distribution of functional epitopes across the SARS-CoV-2 genome is plotted. Each bar represents one validated epitope. The X-axis shows its position in the SARS-CoV-2 ORFeome (open reading frame-ome). The bar fill indicates its MHC restriction, and the height of the bar indicates the fraction of MHC-matched patients recognizing the epitope. Patients were considered positive for an epitope if the aggregate performance of the epitope in the screen data exceeded a threshold (mean+2SD of the enrichment of all SARS-CoV-2 fragments in the healthy controls). For clarity, overlapping epitopes are plotted as adjacent bars.

FIG. 14D shows that ORF1ab has the most epitopes among all the SARS-CoV-2 ORFs. The number of epitopes for each SARS-CoV-2 ORF is plotted. The stacked bar graphs show the number of immunodominant epitopes per ORF, with the bar fills indicating the MHC restriction of each epitope. The MHC fill-coding is the same as that of FIG. 14C.

FIG. 15 shows that UVCs express a high level of NK ligand MICA but do not express any MHC-I. Panel A shows a flow cytometry analysis of NK ligand MICA in the UVC and a parent induced pluripotent stem cell (iPSC). The X-axis shows the fluorescent intensity of the MICA protein. The Y-axis shows the number of cells. The area fill indicating the type of cells is shown on the right of the plot. Panel B shows a flow cytometry analysis of expression of MHC-I in the UVC and the parental iPSC. The X-axis shows the fluorescent intensity of MHC-1 (HLA subtype A, B, or C). The Y-axis shows the number of cells.

FIG. 16 shows that MHC-I deficient UVCs trigger robust cell lysis by monkey NK cells in vitro. A flow cytometry-based natural killer cytotoxicity assay involving Calcein AM (CAM) staining was used to measure the amount of cytotoxicity in the UVCs in the presence of the NK cells. The X-axis shows the effector-to-target (E:T) ratios. The Y-axis shows the percentage (%) of the NK cell cytotoxic activity. MHC-I deficient endothelial cells with (KO EC) showed higher cytotoxicity when mixed with the Macaque NK cells, when compared to that of the wildtype ECs (WT EC). Both KO EC and WT EC were differentiated from the UVC iPSCs.

FIG. 17A and FIG. 17B show that additional NK ligands increase the NK cell response to MHC-I deficient UVCs. FIG. 17A shows that the additional NK ligands increase the expression of cytokines in the NK cells in response to the MHC-I deficient UVCs. Intracellular cytokine staining assays was used to measure the expression of CD107a, MIP-β, IFN-γ, or TNF-α in the NK cells. The summary of all responding NK cells is also shown on the far-right. The X-axis lists the KO-UVCs expressing no ligands (KO), MICA (KO-MICA), MICB, (KO-MICB), or ULBP1 (KO-ULBP1). The Y-axis shows the percentage (%) of NK cells responsive to the KO-UVC. Each point represents an individual animal tested. FIG. 17B shows that the additional NK ligands could induce the expression of multiple cytokines in the NK cells. Simplified Presentation of Incredibly Complex Evaluations (SPICE) was used to analyze the multidimensional cytokine responses of the NK cells. The pie arc and pie chart fill legends are shown at the bottom.

FIG. 18A and FIG. 18B show robust expression of the SARS-CoV-2 spike protein in the UVC iPSCs. FIG. 18A shows that about half of the UVC iPSCs expressed the spike protein. A flow cytometry analysis was used to measure the expression of the SARS-CoV-2 spike protein. The X- and Y-axis show the fluorescent staining intensity of the spike protein and the forward scatter height FSC-H, respectively. About 48.5% of MHC-I deficient (B2M−/−) IPSCs engineered to express both MICA (MICA+) and the SARS-CoV-2 spike protein (Spike+) had a high level expression of the spike protein, as compared to only about 0.41% of the control IPSCs engineered to only express MICA. FIG. 18B shows that the expression level of the spike protein in the UVC iPSCs was comparable to that in HEK293 cells transiently transfected with a spike protein expression plasmid. A cell surface flow analysis was used to measure the expression of the SARS-CoV-2 spike protein in HEK293 cells and UVC iPSCs. The X- and Y-axis show the fluorescent staining intensity of the spike protein and the number of cells, respectively.

FIG. 19A and FIG. 19B show the results of an antibody ELISA at weeks 0, 2, 6, and 8 post vaccination with UVC expressing a SARS-CoV-2 spike protein or receptor binding domain thereof, for 6 monkeys, for both the receptor binding domain of the SARS-CoV-2 spike protein (FIG. 19A) or full length spike protein (FIG. 19B), which demonstrates functional testing of the UVC in an NHP model.

FIGS. 20A-20J show CRISPR genetic engineering of an iPS cell line to create a hyper-immunogenic, self-adjuvanting cellular vaccine. FIG. 20A shows a schematic of the Universal vaccine cell CRISPR genetic engineering strategy to create an apoptotic cellular vehicle for antigen delivery. FIG. 20B shows representative flow-cytometric analysis graphs showing expression of SARS-CoV-2 WA1/2020 spike protein on the cell surface. FIG. 20C shows a Western blot showing Spike protein within UVC whole-cell lysates. FIG. 20D shows a graph of ELISA quantification of spike protein released upon UVC lysis. FIG. 20E shows a graph of the proportion of apoptotic cells at 24- and 72-hours post-irradiation as measured by 7-AAD staining and flow cytometry. FIG. 20F shows images of cell morphology, observed by light microscopy, of engineered UVC during expansion culture, and cells reseeded into culture 24 hours after irradiation, showing robust apoptosis and cell death. FIG. 20G shows a graph of the absence of detectable proliferation of irradiated UVC as determined by CellTrace™ Yellow proliferation dye staining and measuring the dilution of the dye by flow cytometry over 72-hours. FIG. 20H shows graphs of representative flow-cytometric analysis showing deletion of MHC class-I and overexpression of MICA on the UVC surface by CRISPR engineering. FIG. 20I shows a graph of cell counts showing exponential expansion of live engineered UVC over 21-days in culture. FIG. 20J shows a graph of the relative expression of pluripotency and self-renewal genes by UVC and the control iPS cells from they were derived, as measured by quantitative-PCR, showing maintenance of an iPS cell gene expression prolife after genetic engineering and expansion. Error bars represent mean+/−SEM.

FIGS. 21A-21D show self-adjuvancy and Enhanced cytolysis of genetically engineered UVC iPS cells via engineered MHC-I deletion and NK apoptotic ligand expression. FIG. 21A shows a graph of the CRISPR knockout of B2M and loss of MHC class-I enhances the killing of UVC cells by human primary NK cells, showing equivalent levels of cytolysis seen with the MHC class-I deficient K562 cell line. FIG. 21B shows elevated cytolysis of MHC deficient UVC cells is observed with macaques NK cells. FIGS. 21C-21D demonstrate the when overexpressed transiently on the UVC, NKG2G family ligands show no elevation in markers of NK cell activation by macaque NK cells, except MICA which significantly elevates levels of macrophage inflammatory protein-1β (MIP-1β). FIG. 21D shows that when stably overexpressed on the UVC by CRISPR editing, MICA enhanced the NK cell functional responses as measured by ICS. **P>0.01, Error bars represent mean+/−SEM.

FIGS. 22A-22E show humoral immune responses in UVC vaccinated macaques. FIG. 22A shows the vaccination schedule for macaques that received a high WA1/2020 spike expressing UVC prime dose (1×10⁸) or low UVC prime dose (1×10⁷) at week 0; and a boost dose of high or low dose matched to that of the prime dose at week 6. FIGS. 22B-22E shows titers for humoral immune responses. Humoral immune responses were assessed at 2-week intervals up to week-10 and then again at weeks 24 and 26 by Spike (FIG. 22B), RBD-specific binding antibody ELISA (FIG. 22C), and pseudovirus neutralization assays (FIG. 22D). FIG. 22E shows that in addition to the WA1/2020 SARS-CoV-2 variant, detectible neutralizing antibodies against the B.1.351 (Beta) and B.1.617.2 (Delta) variants were observed in immunized macaques at weeks 24 and 26. Red bars reflect median responses. Dotted lines reflect assay limit of quantification. NAb, neutralizing antibody.

FIGS. 23A-23E show viral loads in UVC vaccinated macaques after heterologous SARS-CoV-2 challenge. FIG. 23A shows the vaccination schedule for Rhesus macaques immunized with 1×10⁸ WA1/2020 spike expressing UVC at week 0 and received a boost dose of 1×10⁸ matched UVC at week 4. Macaques were then challenged at week 6 by the intranasal and intratracheal routes with 1.0×10⁵ TCID₅₀ of SARS-CoV-2 B.1.617.2 (Delta). Log₁₀[sgRNA (copies per ml)] (limit of quantification 50 copies per ml) were assessed. FIG. 23B shows a graph of peak viral loads in bronchoalveolar lavage (BAL) samples, in sham controls and vaccinated macaques after challenge. FIG. 23C shows a graph of peak viral loads in nasal swabs (NS), in sham controls and vaccinated macaques after challenge. FIGS. 23D and 23E show graphs of viral loads assessed every 2 days in sham and UVC-treated macaques. Dotted lines reflect assay limit of quantification. NAb, neutralizing antibody.

FIGS. 24A-FIG. 24B: FIG. 24A shows the mechanism of action of UVC to replicate natural physiologic immunity and polyvalent UVC design for emerging variants (Omicron). FIG. 24B shows a schematic depiction of the UVC design to deliver a polyvalent SARS-Cov-2 B.1.1.529 Omicron variant vaccine, incorporating the Spike, Nucleocapsid, ORF3a and Membrane viral proteins.

FIG. 25A demonstrates design of a gene targeting vector to insert the SARS-CoV-2 Spike gene into the UVC genome by CRISPR-mediated homology-directed repair. The amino acid sequence of the WA1/2020 Spike protein is shown highlighted the furin cleavage and proline-stabilizing mutations. Figure discloses SEQ ID NOS 64-66, respectively, in order of appearance.

FIG. 25B shows graphs of humoral immune responses in WA1/2020 spike expressing UVC vaccinated rhesus macaques prior to challenge with SARS-CoV-2 B.1.617.2 (Delta). Antibody responses were assessed at weeks 0, 2, 4, 6 and 8 by pseudovirus neutralization assay and an RBD-specific binding antibody ELISA, and live virus neutralization assays. Red bars reflect median responses. Dotted lines reflect assay limit of quantification. NAb, neutralizing antibody.

FIGS. 26A-26B demonstrate the mechanism of action for a population of UVC-produced extracellular vesicles. FIG. 26A demonstrates ARRDC1 targeting of SARS-CoV2 RNAs encoding for the spike protein to extracellular vesicles and the release of additional extracellular vesicles. FIG. 26B shows that UVC apoptotic bodies containing viral proteins and apoptotic mRNA extracellular vesicles are released when delivered as a vaccine composition. The release of the apoptotic bodies activates the innate and adaptive immune response, including activation of CD8 T cells, dendritic cells, CD4 T cells, and memory B cells that produce antibodies.

FIG. 27 demonstrates a schematic of a representative nucleic acid construct for targeting cellular vesicle delivery of an antigen-encoding RNA. Figure discloses SEQ ID NO: 67.

FIG. 28 shows the centrifugation protocol for isolation of exosomes from universal vaccine cells (UVCs) transfected with an expression vector encoding the construct of FIG. 27.

FIG. 29 shows a Western Blot of positive and negative exosome markers. TSG101 and CD9 are positive exosome markers. Calnexin is a negative exosome marker. Each lane corresponds with a condition as indicated: (Lane 1) exosomes isolated from control cells that were not transfected with ARRDC1; (Lanes 2-4) exosomes isolated from untreated cells transfected with ARRDC1; (Lanes 5-7) exosomes isolated from irradiated cells transfected with ARRDC1; and (Lane 8) a cell pellet control sample.

FIG. 30 shows flow cytometry graphs of CD63 and TSG101 expression in exosomes isolated from unstained, control, and ARRDC1 transfected cells.

FIG. 31 shows cytometry graph of SAR-CoV2-Spike expression in isolated extracellular vesicles from control, cells transfected with spike mRNA, and cell transfected with spike mRNA and treated with cycloheximide.

FIG. 32 shows a flow cytometry graph of SAR-CoV2-Spike expression in cells co-cultured with EVs with spike mRNA, EVs with spike mRNA and cycloheximide, and control cells that were not co-cultured with EVs.

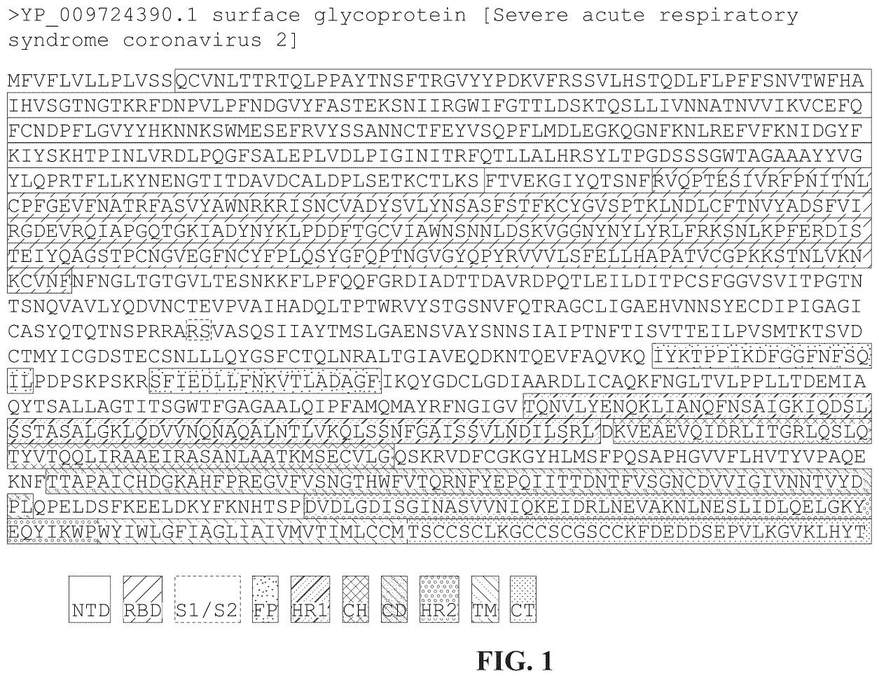

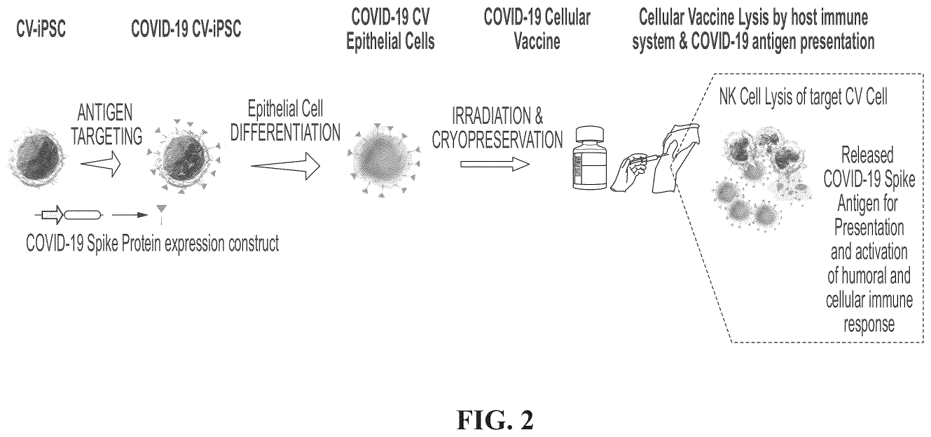

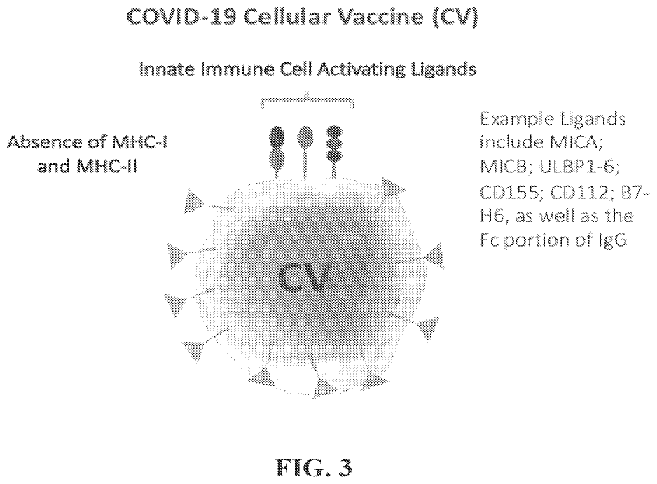

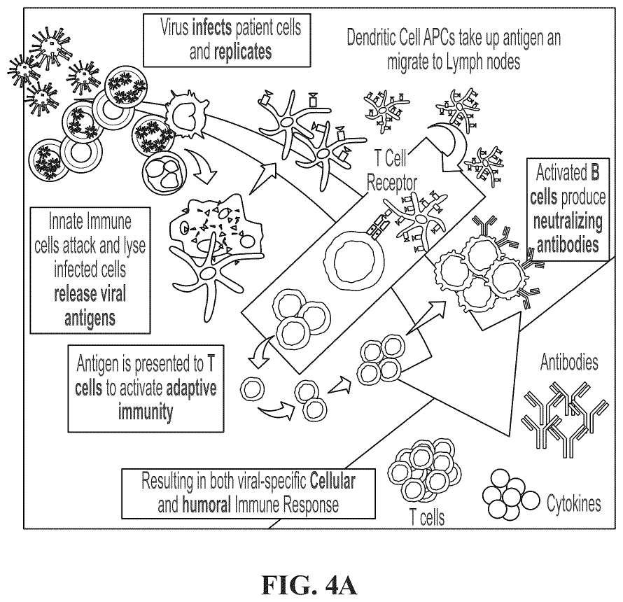

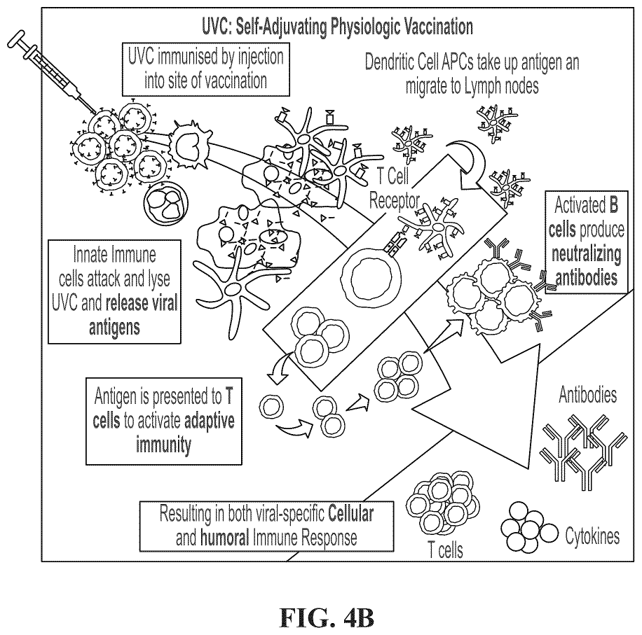

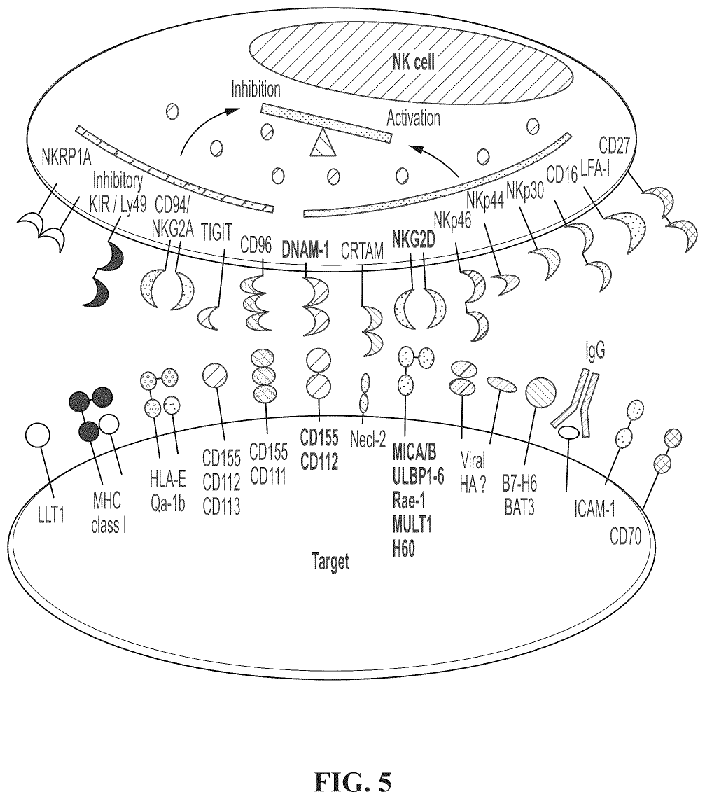

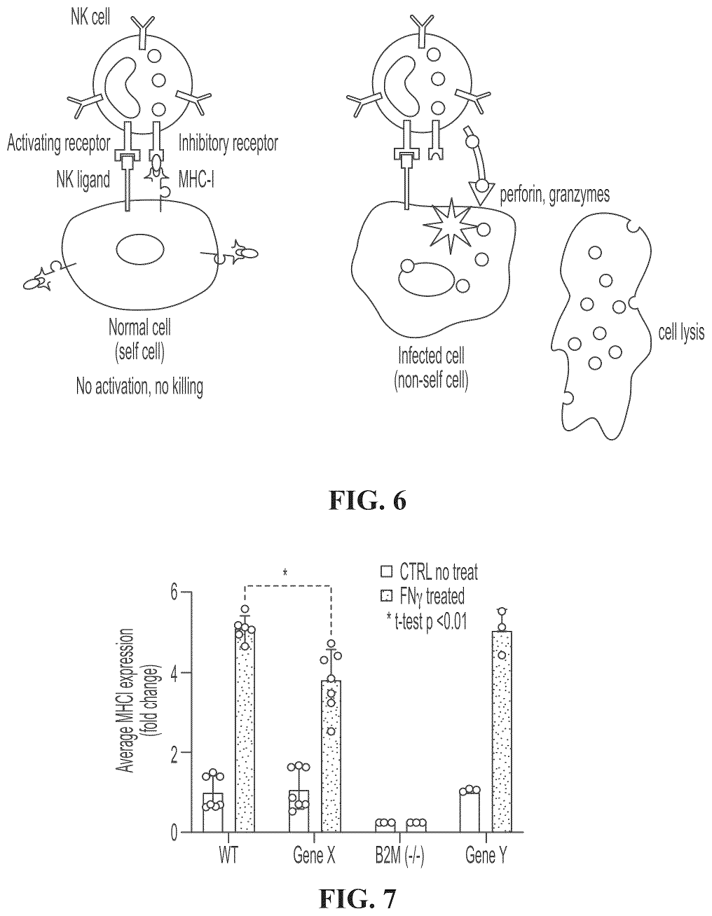

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### DETAILED DESCRIPTION

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

### Definitions

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document. All references disclosed herein, including patent references and non-patent references, are hereby incorporated by reference in their entirety as if each was incorporated individually. However, where a patent, patent application, or publication containing express definitions is incorporated by reference, those express definitions should be understood to apply to the incorporated patent, patent application, or publication in which they are found, and not necessarily to the text of this application, in particular the claims of this application, in which instance, the definitions provided herein are meant to supersede.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein, “optional” or “optionally” means that the subsequently described circumstance may or may not occur, so that the description includes instances where the circumstance occurs and instances where it does not.

The terms “about” or “approximately” and their grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%, 5%, 4%, 3%, 2%, or 1% from that value.

The term “vaccine” and its grammatical equivalents as used herein refer to an agent that elicits a host immune response to an infectious disease.

The term “cellular vaccine” and its grammatical equivalents as used herein refer to a vaccine agent that utilizes cells to expose antigens to the host immune system.

The term “target cell” or “target cell line” and their grammatical equivalents as used herein refer to a selected cell line described herein as the carrier of a certain type of pathogen antigens.

The term “activation” or “activating” and its grammatical equivalents as used herein can refer to a process whereby a cell transitions from a resting state to an active state.

The term “antigen” and its grammatical equivalents as used herein refer to a molecule that contains one or more epitopes or binding sites capable of being bound by one or more receptors or antibodies. For example, an antigen can stimulate a host's immune system to elicit a cellular antigen-specific immune response or a humoral antibody response when the antigen is presented. An antigen can also have the ability to elicit a cellular and/or humoral response by itself or when present in combination with another molecule or other molecules.

An “engineered cell” and its grammatical equivalents as used herein refer to a cell that comprises an exogenous nucleic acid or amino acid sequence; or contains an alteration, addition, or deletion in an endogenous nucleic acid sequence.

The “innate immune system” as discussed herein refers to the first line of defense against non-self pathogens is the innate, or non-specific, immune response of a subject. The innate immune response consists of physical, chemical and cellular defenses against pathogens. “Innate immune cell” as described herein refers generally to a phagocytic or cytolytic immune cell involved in the innate immune response. Specifically, these phagocytic or cytolytic immune cells include monocytes (which develop into macrophages), macrophages, neutrophils, eosinophils, basophils, and Natural killer (NK) cells, and mast cells.

The term “construct” and its grammatical equivalents as used herein refer to a macromolecule or complex of molecules comprising a polynucleotide to be delivered to a host cell, either in vitro or in vivo.

The term “vector” and its grammatical equivalents as used herein refer to any nucleic acid construct capable of directing the delivery or transfer of a foreign genetic material to target cells, where it can be replicated and/or expressed. The term “vector” as used herein comprises the construct to be delivered. A vector can be a linear or a circular molecule. A vector can be integrating or nonintegrating.

The term “integration” and its grammatical equivalents as used herein refer to one or more nucleotides of a construct that is stably inserted into the cellular genome, i.e., covalently linked to the nucleic acid sequence within the cell's chromosomal DNA.

The term “transgene” and its grammatical equivalents as used herein refer to a gene or genetic material that is transferred into a cell. For example, a transgene can be a stretch or segment of DNA containing a gene that is introduced into a cell. A transgene can retain its ability to produce RNA or polypeptides (e.g., proteins) in an engineered cell. A transgene can be composed of different nucleic acids, for example RNA or DNA. A transgene can comprise recombination arms. A transgene can comprise engineered sites.

The term “CRISPR”, “CRISPR system,” or “CRISPR nuclease system” and their grammatical equivalents can include a non-coding RNA molecule (e.g., guide RNA) that binds to DNA and Cas proteins (e.g., Cas9) with nuclease functionality (e.g., two nuclease domains). See, e.g., Sander, J. D., et. al, “CRISPR-Cas systems for editing, regulating and targeting genomes,” Nature Biotechnology, 32:347-355 (2014); see also e.g., Hsu, P. D., et al., “Development and applications of CRISPR-Cas9 for genome engineering,” Cell 157(6): 1262-1278 (2014).

The term “sequence” and its grammatical equivalents as used herein refer to a nucleotide sequence, which can be DNA or RNA; can be linear, circular or branched; and can be either single stranded or double stranded. A sequence can be mutated. A sequence can be of any length, for example, between 2 and 1,000,000 or more nucleotides in length (or any integer value there between or there above), e.g., between about 100 and about 10,000 nucleotides or between about 200 and about 500 nucleotides.

As used herein, the terms “reprogramming” or “dedifferentiation” or “increasing cell potency” or “increasing developmental potency” refers to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

As used herein, the term “differentiation” is the process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the term “induced pluripotent stem cells” or “iPSCs”, means that the stem cells are produced from differentiated adult, neonatal or fetal cells that have been induced or changed, i.e., reprogrammed into cells capable of differentiating into tissues of all three germ or dermal layers: mesoderm, endoderm, and ectoderm. The iPSCs produced do not refer to cells as they are found in nature.

As used herein, the term “Universal Vaccine Cell” “UVC” refers to a vaccine composition described herein. A vaccine composition can comprise a cell provided herein.

As used herein, the term “embryonic stem cell” refers to naturally occurring pluripotent stem cells of the inner cell mass of the embryonic blastocyst. Embryonic stem cells are pluripotent and give rise during development to all derivatives of the three primary germ layers: ectoderm, endoderm and mesoderm. They do not contribute to the extraembryonic membranes or the placenta, i.e., are not totipotent.

As used herein, the term “multipotent stem cell” refers to a cell that has the developmental potential to differentiate into cells of one or more germ layers (ectoderm, mesoderm and endoderm), but not all three. Thus, a multipotent cell can also be termed a “partially differentiated cell.” Multipotent cells are well known in the art, and examples of multipotent cells include adult stem cells, such as for example, hematopoietic stem cells and neural stem cells. “Multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, the term “multipotency” refers to a state of a cell with a degree of developmental potential that is less than totipotent and pluripotent.

Pluripotency can be determined, in part, by assessing pluripotency characteristics of the cells. Pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4, NANOQ SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

Two types of pluripotency have previously been described: the “primed” or “metastable” state of pluripotency akin to the epiblast stem cells (EpiSC) of the late blastocyst, and the “Naive” or “Ground” state of pluripotency akin to the inner cell mass of the early/preimplantation blastocyst. While both pluripotent states exhibit the characteristics as described above, the naive or ground state further exhibits: (i) pre-inactivation or reactivation of the X-chromosome in female cells; (ii) improved clonality and survival during single-cell culturing; (iii) global reduction in DNA methylation; (iv) reduction of H3K27me3 repressive chromatin mark deposition on developmental regulatory gene promoters; and (v) reduced expression of differentiation markers relative to primed state pluripotent cells. Standard methodologies of cellular reprogramming in which exogenous pluripotency genes are introduced to a somatic cell, expressed, and then either silenced or removed from the resulting pluripotent cells are generally seen to have characteristics of the primed-state of pluripotency. Under standard pluripotent cell culture conditions such cells remain in the primed state unless the exogenous transgene expression is maintained, wherein characteristics of the ground-state are observed.

A “pluripotency factor,” or “reprogramming factor,” refers to an agent capable of increasing the developmental potency of a cell, either alone or in combination with other agents. Pluripotency factors include, without limitation, polynucleotides, polypeptides, and small molecules capable of increasing the developmental potency of a cell. Exemplary pluripotency factors include, for example, transcription factors and small molecule reprogramming agents.

As used herein, the term “pluripotent stem cell morphology” refers to the classical morphological features of an embryonic stem cell. Normal embryonic stem cell morphology is characterized by being round and small in shape, with a high nucleus-to-cytoplasm ratio, the notable presence of nucleoli, and typical inter-cell spacing.

The term “effective amount” or “therapeutically effective amount” refers to an amount that is sufficient to achieve or at least partially achieve the desired effect.

### Overview

Provided herein are compositions, kits, methods, and uses thereof for inducing an immune response to a microorganism or a tumor. Briefly, further described herein are (1) platforms; (2) cell modifications include ARRDC1 genomic modifications, HLA gene modification, and cell surface protein modifications; (3) methods of genetic modification; (4) cell compositions; (5) vaccine cell differentiation and expansion; (6) antigen expression constructs; (7) methods of vaccinating a subject; and (8) kits.

Provided herein is a vaccine platform based on a genetically engineered human stem cell, termed the Universal Vaccine Cell (UVC), wherein the genetic engineering is for instance by use of a nuclease mediated ex vivo gene editing system such as a CRISPR system.

One feature of this vaccine platform is that it reproduces physiologic immunity that is engendered naturally through lytic viral infection and the resulting apoptosis of primary human cells. Embodiments of the platform are designed to deliver desired antigenic payload, including embodiments with plurality of antigens from potentially a plurality of pathogens, within the context of a physiological apoptotic environment, to both release abundant antigen and simultaneously stimulate the host immune response. In exemplary embodiments disclosed herein, the SARS-CoV-2 virus has been used as a rigorous and timely test platform, to demonstrate that this self-adjuvanting, polyvalent UVC, can generate a robust and antigen-specific humoral immune response in vaccinated macaques. This hyper-immunogenic vaccine resulted in reduced viral loads in animals challenged with heterologous SARS-CoV-2 variant, which recapitulates the current experience of a population vaccinated against the initial Wuhan variant, yet now exposed to novel variants like Delta and Omicron.

The present disclosure provides, inter alia, a novel cellular vaccine platform that offers distinct advantages over current systems that enable the development of robust, safe, and highly scalable cellular vaccines for any pathogen. Standard vaccines that are used to vaccinate against microbes utilize viruses, lipid nanoparticles, or nucleic acids. Unlike these standard vaccines, cellular vaccines provides the distinct advantage of delivering the immunogenic antigen in a physiologically relevant way, enabling the host immune cell to engage with the antigen as it would if the subject was naturally infected. In addition, the cellular component is likely to act as an intrinsic adjuvant via the in vivo creation of apoptotic bodies that will stimulate, attract and recruit cells of the innate system to facilitate a robust immune response and development of immunological memory. The cellular vaccine actually “mimics” the natural process of immune cell lysis of infected cells, and therefore the antigen is delivered to the immune system in the exact same way as it would be via a naturally acquired immunity to invading pathogen. Thus, some embodiments are self-adjuvanting.

Cancer cell line based cellular vaccines are currently in development. However, unlike these other cellular vaccines, the present disclosure provides novel cellular vaccines that are genetically engineered, which enables the precise creation of the “ideal” target cell that is designed to the killed; as opposed to a natural quirk of a cancer cell line biology. The cell surface receptors expressed by the target cell are specifically designed for a “targeted” lysis by defined cells of the host innate immune system (i.e., absence of MHC and gain of the “missing-self” signal, and targeted expression of “kill-me” signals for cytolytic and phagocytic cells).

Embodiments of the present disclosure provide a cellular vaccine platform that utilizes a stem cell (e.g., an induced pluripotent stem cell) that can be differentiated in vitro. The use of stem cells (e.g., iPSCs) is beneficial, as it avoids having to use any type of transformed cancer cell, while retaining the ability to perpetually grow a stock of engineered vaccine to massive scale production of a stable and consistent cell product. Differentiation into a defined, terminally differentiated and stable cell lineage (such as epithelial cells or skin dendritic “Langerhans” cells) allows the vaccine to move even further away from a cancer-type cell to engineer the same cell type as that from the recipient tissue where it will be delivered.

Further provided herein are vaccines comprising genetically engineered cells differentiated into an epithelial (dendritic) Antigen Presenting Cell (APC) from a stem cell (e.g., an iPSC) such that there is “APC mimicry.” Some embodiments of the vaccine comprise an MHC null and NK/Mo Innate Immunity+ APC being presented to the host patient's native MHC specific APC/innate immune system. As such, the vaccine should produce a superior and safer immune antigenic response and naturalizing Ab production to confer a lasting immunity post intradermal/SQ injection in to the skin and the frontline site of APC in the body post the vaccine injection of our Universal Vaccine Cell (UVC).

Further provided herein are vaccine compositions comprising genetically engineered human cells and/or extracellular vesicles produced by the genetically engineered human cells. Extracellular vesicles are lipid particles that are released cells that function to transfer cellular proteins and nucleic acids to other cells. Extracellular vesicles are not able to replicate but serve as cell messengers that can enhance both innate and adaptive immune responses when released from a UVC described herein. The UVCs provided herein can be genetically engineered to produce extracellular vesicles with antigen encoding mRNAs targeted to the extracellular vesicles as payload. Apoptosis and phagocytosis of the extracellular vesicles by the host immune system's dendritic cells promote translation of the antigen encoding mRNAs and promote antigen presentation. In turn, the antigen is recognized by CD4+ T cells that activate B cell production of neutralizing antibodies to the antigen. The vaccine compositions provided herein have dual vaccine responses including but not limited to neutralizing antibody production to the UVC protein antigen and T cell responses to the mRNA targeted to extracellular vesicles.

Table 1 provides a further comparison between cellular vaccine and viral vector based vaccine, and exemplar benefits of cell based vaccines.

TABLE 1
Cellular Vaccine vs. Viral Vector Based Vaccine

| | | Universal Vaccine | | |
|---|---|---|---|---|
| | | Cell and | Viral Vector | |
| | Universal | Extracellular | Based | |
| | Vaccine Cell | vesicles | Vaccine | mRNA Vaccine |
| Vaccine | Live physiologic | Live physiologic | Non-replicating | Synthetic non- |
| Delivery Vehicle | Antigen | Antigen Presenting | adenovirus; | physiological |
| | Presenting Cell; | Cell; Live | e.g., AD26 | nanoparticle |
| | Live mammalian | mammalian cell; | | |
| | cell | and/or extracellular | | |
| | | vesicles produced by | | |
| | | the live cells | | |
| Immunogenic | Full length proteins | Full length proteins | Epitope | mRNA |
| Antigen | (knowledge of | (knowledge of | fragments and | expressing viral |
| | precise immunogenic | precise immunogenic | predetermined | antigen protein |
| | epitope not needed) | lepitope not needed) | immunogenic | |
| | | and nucleic acid | sequences | |
| | | encoding | required | |
| | | immunogenic | | |
| | | antigens targeted to | | |
| | | extracellular vesicles | | |
| Antigen | Mimics physiological | Mimics physiological | Presentation | Antigen expression |
| Presentation | lytic stage of viral | lytic stage of viral | via decoy viral | and presentation |
| Method | infection | infection and | infection | limited by |
| | | enhances CD4+ T | | Nanoparticle |
| | | cell and B cell | | immunogenicity |
| | | recruitment | | |
| Antigen Density | Multiple Copies of | Multiple copies of | Multiple Copies of | Multiple Copies of |
| | Antigenic Viral | antigenic viral | Antigenic Viral | Antigenic Viral |
| | Epitope per Cell | epitope per cell | Epitope per Cell | Epitope per Cell |
| Adjuvant | Self-Adjuvanting; | Self-Adjuvanting; | Viral Vector | Theoretically |
| Immunogenicity | highly efficient | highly efficient | Immunogenicity, | less immunogenic |
| | physiologic antigen | physiologic antigen | potential | and efficient |
| | presentation to | presentation to | for NAB's to | antigen |
| | enhance | enhance | Viral Vector | presentation to |
| | immunogenicity | immunogenicity and | | host APC |
| | Engineered NK | immune memory of | | |
| | Target and HLA | the immunogenic | | |
| | null phenotype to | antigen | | |
| | enhance cell lysis | | | |
| | and immunogenicity | | | |
| Commercial | Stable cell line, | Stable cell line, | Two-stage | Efficient low |
| Scale | single stage large | single stage large | bioreactor | cost, large |
| | scale manufacturing | scale manufacturing | manufacturing | volume scale |

As shown above, the vaccine compositions provided herein deliver both protein antigen and/or antigen-encoding nucleic acids (e.g., mRNA) that enable antigen presentation of the exogenous protein antigen and presentation of endogenously expressed antigens in a single vaccine. The vaccine compositions provided herein can also provide protection from multiple types of infections when the UVC is engineered to present protein antigens and release extracellular vesicles with RNA payloads that encode one or more protein antigens.

### (1) Platform

Provided herein is, inter alia, a vaccine platform that comprises genetically modified platform cells for use in vaccines. Specifically, platform cells described herein are stem cells such as embryonic stem cells or pluripotent stem cells that are genetically modified by disruption of one or more MHC genes (or specifically in the case of human cells, HLA genes) to facilitate use as an allogeneic vaccine platform. The platform cell described herein can be modified to express an exogenous protein or antigenic fragment thereof relevant for a specific vaccine tailored to specific antigens such as viral antigens.

In some embodiments, the platform cell comprises a genomic disruption in at least one human leukocyte antigen (HLA) gene or at least one transcriptional regulator of an HLA gene; and expresses an exogenous protein that binds to a phagocytic or cytolytic immune cell, for instance an innate immune cell, and stimulates activity (e.g., phagocytosis, cytolytic activity, proinflammatory cytokine secretion) of the immune cell. In some embodiments, the platform cell expresses a secretory exogenous protein that attracts a phagocytic or cytolytic immune cell to the platform cell (or vaccine cell designed from the platform cell). In some embodiments, the platform cell expresses and secretes or presents on its surface an exogenous cell surface protein that binds to a phagocytic or cytolytic immune cell.

### Platform Cells

Provided herein is a platform for a vaccine composition comprising an engineered cell or a population thereof. In some embodiments, the cells are mammalian. In some embodiments, the cells are human. In some embodiments, the cells are murine or non-human primate cells. In some embodiments, the platform cells described herein are engineered stem cells. In some embodiments, the engineered stem cells are human stem cells. In some embodiments, the stem cell is an induced pluripotent stem cell (iPSC), an embryonic stem cell (ESC), an adult stem cell (ASC), a pluripotent stem cell (PSC), or a hematopoietic stem and progenitor cell (HSPC). In some embodiments, the stem cell is an induced pluripotent stem cell (iPSC). In some cases, a cell such as an iPS can be differentiated into an epithelial (ectoderm derived iPS), APC (Langerhans, dendritic cell) or combinations thereof. In some embodiments, the cells are immune cells or leukocytes. In some embodiments, the cells are peripheral blood mononuclear cells (PBMCs). In some embodiments, the cells are T cells. In some embodiments, the cells are Natural Killer (NK) cells.

### Extracellular Vesicles

A platform cell described herein can be engineered to produce extracellular vesicles that comprise a nucleic acid. In some embodiments, the nucleic acid encodes for an antigen or a fragment thereof. In some embodiments, the nucleic acid is an RNA. In some embodiments, the RNA is a messenger RNA (mRNA). In some embodiments, the RNA is a microbial RNA. In some embodiments, the antigen is a microbial antigen or protein. In some embodiments, the antigen is a tumor antigen. In some embodiments, the RNA promotes an immune response in a subject. In some embodiments, the extracellular vesicle comprises a viral RNA, a fungal RNA, a parasitic RNA, or a bacterial RNA or any combination thereof. In some embodiments, the extracellular vesicle comprises a viral RNA, wherein the viral RNA is an RNA encoding for a spike protein, a nucleocapsid protein, or a glycoprotein. In some embodiments, the extracellular vesicle comprises a viral RNA or microbial RNA that is engineered or derived from a severe acute respiratory syndrome-related (SARS) coronavirus (CoV), an influenza virus, an Epstein-Barr virus (EBV), a megavirus, a Norwalk virus, a Coxsackie virus, a middle east respiratory syndrome (MERS)-related coronavirus, a SARS-Cov-2 virus, a hepatitis B virus, a varicella zoster virus, a parvovirus, an adenovirus, a Marburg virus, an Ebola virus, a Rabies virus, a Smallpox virus, a human immunodeficiency virus (HIV), a Hantavirus, a Dengue virus, a rotavirus, a MERS-CoV, a mumps virus, a cytomegalovirus (CMV), a Herpes virus, a papillomavirus, a Chikungunya virus, a respiratory syncytial virus (RSV), a variant, or any combination thereof. In some embodiments, the microbial RNA or viral RNA encodes for a SARS-CoV2 spike protein. In some embodiments, the microbial RNA or viral RNA encodes for an RSV glycoprotein.

In some embodiments, the extracellular vesicle is an arrestin domain containing protein 1 [ARRDC1]-mediated microvesicle (ARMM), an exosome, an ectosome, a macrovesicle, a microparticle, an apoptotic body, a vesicular organelle, an oncosome, an exosphere, an exomere, or a cell-derived nanovesicle (CDN), or a liposome.

In some embodiments, a genetically engineered human cell provided herein comprises a genomic modification that results in overexpression of at least a portion of a human arrestin domain containing protein 1 [ARRDC1] or a functional variant thereof. In some embodiments, the overexpression is in an amount sufficient to result in increased vesicle formation as compared to a comparable cell that lacks the genomic modification.

Arrestin domain containing protein 1 (ARRDC1) is expressed by extracellular vesicles that are distinct from exosomes called arrestin domain containing protein 1 [ARRDC1]-mediated microvesicles (ARMMs). In contrast to exosomes, the biogenesis of ARMMs occurs at the plasma membrane. Overexpression of the ARRDC1 protein in cells increases the production of ARMMs. Endogenous proteins (e.g., cell surface receptors) and exogenous proteins or RNAs provided herein (e.g., microbial RNAs or fragments thereof) are actively recruited into ARMMs and can be delivered into recipient cells to initiate intercellular communication.

The genetically engineered human cell provided herein that comprises the genomic modification that results in overexpression of at least a portion of a human arrestin domain containing protein 1 [ARRDC1] or a functional variant thereof can be used to produce extracellular vesicles for administration as a vaccine composition and/or an adjuvant to any other vaccine platform provided herein.

The extracellular vesicles can be characterized by protein expression, morphology, and diameter. The properties of an extracellular vesicle can be determined by methods known in the art, e.g, electron microscopy, flow cytometry, proteomics, or fluorescent microscopy. In some embodiments, an extracellular vesicle provided herein is at least about 40 nanometers (nm) in diameter, at least about 50 nm in diameter, at least about 60 nm in diameter, at least about 70 nm in diameter, at least about 80 nm in diameter, at least about 90 nm in diameter, at least about 100 nm in diameter, at least about 110 nm in diameter, at least about 120 nm in diameter, at least about 130 nm in diameter, at least about 140 nm in diameter, at least about 150 nm in diameter, at least about 200 nm in diameter, at least about 300 nm in diameter, at least about 400 nm in diameter, at least about 500 nm in diameter, at least about 600 nm in diameter, at least about 700 nm in diameter, at least about 800 nm in diameter, at least about 900 nm in diameter, or at least about 1000 nm or 1 micrometer (μm) in diameter. In some embodiments, an extracellular vesicle provided herein is between at least about 40 nm in diameter up to 1000 nm in diameter. In some embodiments, an extracellular vesicle provided herein is between at least about 50 nm in diameter up to 400 nm in diameter. In some embodiments, an extracellular vesicle provided herein is between at least about 100 nm in diameter up to about 400 nm in diameter.

Extracellular vesicles provided herein can be administered with a UVC provided herein, produced by a UVC provided herein, and/or isolated from a population of genetically engineered human cells provided herein. Extracellular vesicles can be isolated from a cell culture medium by centrifugation and subsequently filtered. In some embodiments, an extracellular vesicle provided herein is isolated by precipitation, centrifugation, filtration, immuno-separation, tangential flow, liquid chromatography, and/or flow fractionation. Differential ultracentrifugation is a technique where secreted exosomes are isolated from the supernatants of cultured cells. This approach allows for separation of extracellular vesicles from non-membranous particles, by exploiting their relatively low buoyant density. Size exclusion allows for their separation from biochemically similar, but biophysically different microvesicles, which possess larger diameters of up to 1,000 nanometers. Differences in floatation velocity further allows for separation of differentially sized extracellular vesicles (e.g., ARMMs). Further purification of an extracellular vesicle provided herein can rely on specific properties of the particular extracellular vesicle of interest. This includes, for example, use of immunoadsorption with a protein or to select specific vesicles with RNA encoding an antigen provided herein (e.g., a microbial RNA or a fragment thereof).

In some embodiments, an extracellular vesicle provided herein is isolated by centrifugation. Exemplary centrifugal force needed to isolate an extracellular vesicle provided herein includes, e.g., increasing centrifugal force from 2000×g to 10,000×g to separate the medium- and larger-sized particles and cell debris from the extracellular vesicle pellet at 100,000×g. Enhanced specificity of extracellular vesicle purification can deploy sequential centrifugation steps in combination with ultrafiltration, or equilibrium density gradient centrifugation in a sucrose density gradient, to provide for the greater purity of the extracellular vesicle preparation (flotation density 1.1-1.2 g/ml) or application of a discrete sugar cushion in preparation.

Other chemical methods have exploit differential solubility of extracellular vesicles for precipitation techniques, addition to volume-excluding polymers (e.g., polyethylene glycols (PEGs)), possibly combined additional rounds of centrifugation or filtration. For example, a precipitation reagent, ExoQuick®, can be added to conditioned cell media to quickly and rapidly precipitate a population of exosomes. Flow field-flow fractionation (FlFFF) is an elution-based technique that is used to separate and characterize macromolecules (e.g., proteins) and nano- to micro-sized particles (e.g., organelles and cells) and which has been successfully applied to fractionate extracellular vesicles from culture media.

Extracellular vesicles provided herein can be characterized by antibody immunoaffinity techniques to identify extracellular vesicle-associated antigens. Conjugation to magnetic beads, chromatography matrices, plates or microfluidic devices allows isolating of specific extracellular vesicle populations related to their production from a parent UVC or associated cellular regulatory state. Other affinity-capture methods use lectins which bind to specific saccharide residues on the extracellular vesicle surface.

The genetically engineered human cells provided herein can be used to produce extracellular vesicles comprising an RNA encoding an antigen of interest in vitro, in vivo, or ex vivo. The vaccine compositions can comprise a population of genetically engineered human cells; a population of extracellular vesicles provided herein; or a combination of genetically engineered human cells and extracellular vesicles provided herein. Cell modifications can be made to a genetically engineered human cell provided herein that modulate the expression of a gene associated with extracellular vesicle formation; human leukocyte antigen genes; and/or cell surface proteins. Cell modifications are discussed further below.

### (2) Cell Modifications

### ARRDC1 Modifications

In some embodiments, a platform cell described herein comprises a genomic modification that results in overexpression of at least one extracellular vesicle gene, wherein the overexpression is in an amount sufficient to result in increased extracellular vesicle formation as compared to a comparable cell that lacks the genomic modification. In some embodiments, the extracellular vesicle gene is arrestin domain containing protein 1 [ARRDC1] or a functional variant thereof. In some embodiments, the extracellular vesicle gene is human arrestin domain containing protein 1 [ARRDC1] or a functional variant thereof. In some embodiments, the ARRDC1 gene encodes for an ARRDC1 polypeptide. An exemplary polypeptide sequence of an ARRDC1

(SEQ ID NO: 55)
MGRVQLFEISLSHGRVVYSPGEPLAGTVRVRLGAPLPFRAIRVTCIGSCG
VSNKANDTAWVVEEGYFNSSLSLADKGSLPAGEHSFPFQFLLPATAPTSF
EGPFGKIVHQVRAAIHTPRFSKDHKCSLVFYILSPLNLNSIPDIEQPNVA
SATKKFSYKLVKTGSVVLTASTDLRGYVVGQALQLHADVENQSGKDTSPV
VASLLQKVSYKAKRWIHDVRTIAEVEGAGVKAWRRAQWHEQILVPALPQS
ALPGCSLIHIDYYLQVSLKAPEATVTLPVFIGNIAVNHAPVSPRPGLGLP
PGAPPLVVPSAPPQEEAEAEAAAGGPHFLDPVFLSTKSHSQRQPLLATLS
SVPGAPEPCPQDGSPASHPLHPPLCISTGATVPYFAEGSGGPVPTTSTLI
LPPEYSSWGYPYEAPPSYEQSCGGVEPSLTPES.

Gene sequences for human ARRDC1 can include, e.g., NCBI Gene ID: 92714 (SEQ ID NO: 56).

An ARRDC1 nucleic acid construct can be used to produce the genomic modification provided herein. The ARRDC1 construct can be delivered to a cell in combination with a nucleic acid encoding an antigen provided herein. In some embodiments, the ARRDC1 construct comprises any one or more of: a transactivator of transcription (Tat); a hairpin RNA; and/or a tetrapeptide motif. The tetrapeptide motif can be used to recruit the nucleic acid encoding for the antigen to interact with proteins that are associated with a particular type of extracellular vesicle. For example, a proline-proline-X-tyrosine (PPXY) tetrapeptide motif can be used to target the nucleic acid to an ARMM, where X is any other amino acid. In some embodiments, the tetrapeptide motif is a PPXY motif, a proline-serine-alanine-proline (PSAP) motif (SEQ ID NO: 68), or a combination thereof. In some embodiments, the ARRDC1 nucleic acid construct comprises: (i) ARRDC1; (ii) one or more tetrapeptide motif; and a (iii) Tat. In some embodiments, the ARRDC1 nucleic acid construct further comprises a hairpin RNA. In some embodiments, the hairpin RNA comprises a stem-loop-containing trans-activating response (TAR) element RNA. In some embodiments, the hairpin RNA (e.g., TAR) is fused to the nucleic acid encoding an antigen. In some embodiments, the hairpin RNA binds to the nucleic acid encoding an antigen.

In some embodiments, the nucleic acid construct provided herein comprises any one or more of the sequences listed in Table 2, a derivative, or a functional fragment thereof.

TABLE 2
Nucleic acid construct sequences.

| Sequence Name | SEQ ID NO: |
|---|---|
| Nucleic acid binding motif: transactivator | SEQ ID NO: 57 |
| of transcription (Tat) Sequence | |
| ARRDC1-Tat Sequence 1 | SEQ ID NO: 58 |
| ARRDC1-Tat Sequence 2 | SEQ ID NO: 59 |
| Hairpin RNA: TAR Sequence | SEQ ID NO: 60 |
| TAR-SARS-CoV2-Spike Sequence | SEQ ID NO: 61 |

### HLA Gene Modifications

In some embodiments, a platform cell described herein comprises a genomic disruption in at least one human leukocyte antigen (HLA) gene or at least one transcriptional regulator of an HLA gene. In some embodiments, the genomic disruption inhibits expression of an HLA protein encoded by the at least one HLA gene on the surface of the cell. In some embodiments, the genomic disruption inhibits expression of an HLA protein encoded by the at least one HLA gene on the surface of the genetically engineered human cell for a period of time sufficient to interact with a protein expressed on the surface of an innate immune cell.

In some embodiments, the genomic disruption results in a reduction in HLA or MHC mediated T cell activation and/or proliferation, for instance, in a subject that is administered an engineered cell described herein or in an ex vivo assay, as compared to another cell expressing the HLA gene. In some embodiments, the genomic disruption results in less HLA or MHC mediated T cell response, for instance, in a subject that is administered an engineered cell described herein or in an ex vivo assay, as compared to a comparable cell lacking the genomic disruption.

In some cases, a platform cell can be a stem cell that is engineered to be HLA deficient. An HLA deficient cell can be HLA-class I deficient, or HLA-class II deficient, or both. In certain embodiments, an HLA deficient cell refers to cells that either lack, or no longer maintain, or have reduced level of surface expression of a complete MHC complex comprising a HLA class I protein heterodimer and/or a HLA class II heterodimer, such that the diminished or reduced level is less than the level naturally detectable by other cells or by synthetic methods.

HLA class I deficiency can be achieved by functional deletion or genomic disruption of any region of the HLA class I locus (chromosome 6p21), or deletion, disruption, or reducing the expression level of HLA class-I associated genes including, not being limited to, beta-2 microglobulin (B2M) gene, TAP 1 gene, TAP 2 gene and Tapasin. In some embodiments, the HLA class I gene disrupted is an HLA-A gene, HLA-B gene, HLA-C gene.

HLA class II deficiency can be achieved by functional deletion, disruption or reduction of HLA-II associated genes including, not being limited to, RFXANK, CIITA, RFX5 and RFXAP. In some embodiments, the HLA class II gene disrupted is an HLA-DP gene, HLA-DM gene, HLA-DOA gene, HLA-DOB gene, HLA-DQ gene, HLA-DR gene.

In some embodiments, provided herein are platform cells that are HLA deficient stem cells such as iPSC that are further modified by introducing genes expressing proteins related but not limited to improved differentiation potential, antigen targeting, antigen presentation, antibody recognition, persistence, immune evasion, resistance to suppression, proliferation, co-stimulation, cytokine stimulation, cytokine production (autocrine or paracrine), chemotaxis, and cellular cytotoxicity, such as non-classical HLA class I proteins (e.g., HLA-E and HLA-G), chimeric antigen receptor (CAR), T cell receptor (TCR), CD 16 Fc Receptor, BCL11b, NOTCH, RUNX1, IL15, 41BB, DAPIO, DAP12, CD24, CD3z, 41BBL, CD47, CD 113, and PD-L1.

In some embodiments, the genetically engineered human cell comprises a genomic disruption in at least one HLA class I gene or the at least one transcriptional regulator of the HLA class I gene and a genomic disruption in at least one HLA class II gene or the at least one transcriptional regulator of the HLA class II gene. In some embodiments, the genetically engineered human cell comprises a genomic disruption in at least one HLA class I transcriptional regulator gene and a genomic disruption in at least one HLA class II transcriptional regulator.

In some embodiments, a platform cell does not express any HLA I proteins on the cell surface. In some embodiments, the cell does not express any HLA II proteins on the cell surface. In some embodiments, the cell does not express any HLA I or HLA II proteins on the cell surface. In some embodiments, a platform cell described herein does not express enough HLA I protein on the cell surface for an immune response to be mounted by a subject if administered to a non-HLA matched subject. In some embodiments, a platform cell does not express enough HLA II protein on the cell surface for an immune response to be mounted by a subject if administered to a non-HLA matched subject.

### Stimulation of Innate Immune Cell Activity

A platform cell described herein is engineered to express an exogenous protein that binds to an innate immune cell such as an NK cell, a dendritic cell, a neutrophil, a macrophage or a mast cell; and stimulates activity (e.g., cytolytic activity, proinflammatory cytokine secretion) of the innate immune cell.

In some embodiments, the platform cell comprises an exogenous nucleic acid encoding for a cell surface protein that binds to a protein expressed on the surface of a phagocytic or cytolytic immune cell. In some embodiments, the platform cell comprises an exogenous nucleic acid encoding for an antigen. In some embodiments, the platform cell comprises an exogenous nucleic acid encoding for a viral protein, a viral antigen, a bacterial protein, a bacterial antigen, a fungal protein, a fungal antigen, a parasitic protein, or a parasitic antigen. In some embodiments, the platform cell comprises an exogenous nucleic acid encoding for a tumor antigen. In some embodiments, the platform cell comprises a nucleic acid that encodes an exogenous protein that binds to an innate immune cell such as an NK cell, a dendritic cell, a neutrophil, a macrophage or a mast cell; and stimulates activity (e.g., trogocytosis) of the innate immune cell.

Innate immune cell activation can be determined by analyzing at least one of degranulation/activation markers (CD107a, CD63, CD107b, CD69) levels of Granzyme B, IFNg, MIP-1b, perform, TNFa, or any combination thereof. Activation can also be determined by imaging, flow cytometry, ELISA, quantitative PCR, or any combination thereof.

### NK Cells:

NK cells express multiple activating and inhibitory receptors that recognize proteins expressed on the surface of other cells. Normal healthy cells express MHC class I molecules on the surface which act as ligands for inhibitory receptors on NK cells and contribute to the self-tolerance of NK cells. Pathogen-infected cells lose surface MHC class I expression, leading to lower inhibitory signals in NK cells. Cellular stress associated with viral infection, such as DNA damage response or senescence program, up-regulates ligands for activating receptors in infected cells. As a result, the signal from activating receptors in NK cell shifts the balance toward NK cell activation and elimination of target cells, directly through NK cell-mediated cytotoxicity or indirectly through secretion of pro-inflammatory cytokines.

In some embodiments, the platform and vaccine cells described herein express one or more NK cell activation ligand. In some embodiments, the platform and vaccine cells described herein are genetically engineered to decrease or eliminate expression of an NK cell inhibition ligand.

Full NK cell activation requires recognition of NK cell activating receptors by one or more NK cell ligand expressed on the surface of a target cell. In some embodiments, platform cells described herein are engineered to enhance their recognition and lysis of the platform cell by NK cells. In some cases, platform cells are engineered to express (or over express) one or more NK cell activating ligand. For example, in one embodiment, the cells can be engineered to express cell MICA/B, Necl-2, or any other ligands listed in Table 3 on the cell surface, or one or more functional domains thereof sufficient to bind an NK cell. In order to express an NK cell activating ligand, or an NK cell binding domain therefrom, the cells can be genetically engineered to introduce an exogenous gene encoding the ligand or domain (e.g., using methods described herein or otherwise known in the art). In some embodiments, a genetically engineered cell described herein expresses at least one ligand of Table 3, or a variant thereof, or domain therefrom.

TABLE 3
NK cell activating receptors and corresponding ligands.

| Receptors | Ligands |
|---|---|
| NKG2D | MICA, MICB, ULBP1-6, Rae-1, MULT1, H60 |
| CD94-NKG2C | HLA-E |
| KIR2DL4 | HLA-G |
| KIR2DS1 | HLA-C2 |
| KIR2DS2 | HLA-C1 |
| KIR2DS3 | — |
| KIR2DS4 | HLA-A11 |
| KIR2DS5 | — |
| KIR3DS1 | HLA-Bw4 |
| NKp30 | B7-H6, BAT3 |
| NKp46 | Heparin, viral HA and HN |
| NKp44 | viral HA and HN, PCNA, proteoglycans |
| CD27 | CD70 |
| LFA-1 | ICAM-1 |
| CD16 | IgG |
| CRTAM | Nectin-like molecules (Necl)-2 |
| DNAM-1 (CD226) | CD155, CD112 (Nectin-2) |

Additionally, NK cells express inhibitory receptors which bind to inhibitory ligands on target cells and inhibit activation of the NK cell. NK cell inhibitory receptors signal through immunoreceptor tyrosine-based inhibitory motifs (ITIMs) present in their cytoplasmic tails. Upon ligand engagement, ITIMs undergo phosphorylation and recruit phosphatases such as Src homology-containing tyrosine phosphatase 1 (SHP-1), SHP-2, and lipid phosphatase SH2 domain-containing inositol-5-phosphatase (SHIP) which further neutralize the activating signals. During NK cell inhibitory signaling, the phosphatases SHP-1 and SHP-2 dephosphorylate the ITAM-bearing Vav-1 molecules and prevent the downstream signaling. Table 4 provides an exemplary list of inhibiting receptors and their corresponding ligands expressed on target cells. Such receptors and ligands are well known in the art.

In some embodiments, the target cells described herein are engineered to enhance their recognition and lysis by NK cells by engineering the cells to not express (or decrease expression of) one or more NK cell inhibitory ligand. For example, in one embodiment, the cells can be engineered to induce a genomic disruption in one or more HLA class I molecule or any other ligands listed in Table 4 on the cell surface. The platform and vaccine cells described herein can be engineered to knockout any one or any combination of genes encoding an NK cell inhibitory ligand, e.g., those listed in Table 4.

TABLE 4
NK cell inhibiting receptors and corresponding ligands.

| Receptors | Ligands |
|---|---|
| Ly49 (murine) | MHC-1 (murine) |
| KIR2DL1 | HLA-C2 |
| KIR2DL2 | HLA-C1 |
| KIR2DL3 | HLA-C1 |
| KIR3DL1 | HLA-Bw4 |
| KIR3DL2 | HLA-A3, -A11 |
| NKR-P1A | LLT1 |
| CD94-NKG2A | HLA-E Qa-1b |
| ILT2 (CD85j) | HLA-A, -B, -C, HLA-G1, HCMA UL18 |
| CD244 (2B4) | CD48 |
| TIGIT | CD155, CD112 (Nectin-2), CD113 |
| CD96 | CD155, CD111 |

### Increase or Mimic Antibody Opsonisation to Increase ARRDC and or Phagocytosis

In some embodiments, a platform cell is opsonized ex vivo in order to mediate increased phagocytosis and/or ARRDC activity in vivo. In some embodiments, a cell is engineered to express additional exogenous proteins on the surface. In some embodiments, the cell is engineered to expresses a high number of exogenous proteins on the cell surface. In some embodiments, the engineered cell is contacted ex vivo with an antibody (e.g., that comprises an antigen binding domain and an Fc domain) that binds to an exogenous protein such that the cell is coated with antibody. The opsonization of the cell can mediate increased phagocytosis and/or ARRDC activity by phagocytes (e.g., macrophages) and NK cells, respectively. In some embodiments, the cell is engineered ex vivo to increase opsonization in vivo. In some embodiments, the cell is engineered to express an Fc domain on the surface of the cell, such that the CH2 domain is proximal to the cell membrane and the CH3 domain is distal to the cell membrane.

### Stimulation of Phagocytosis

In some embodiments, a cell described herein expresses an exogenous protein that binds to a phagocytic cell and stimulates activity (e.g., phagocytosis) of the phagocytic cell. In some embodiments, the cell comprises an exogenous nucleic acid that encodes a protein that binds to a phagocytic cell and stimulates activity (e.g., phagocytosis) of the phagocytic cell. In some embodiments, the phagocytic cell is a macrophage, dendritic cell, eosinophil, or neutrophil. In some embodiments, the exogenous protein is selected from the group consisting of phosphatidylserine, calreticulin, and c1q.

In some cases, cells undergoing apoptosis secrete molecules, so-called “find-me” signals (also referred to as “come-to-get-me” signals), to attract phagocytes toward them. Any and all of these signals can be incorporated into a cell provided herein. Four representative “find-me” signals released by apoptotic cells have been identified, including SIP (sphingosine-1-phosphate), LPC (lysophosphatidylcholine), nucleotides (ATP or UTP) and CX3CL1 (CX3C motif chemokine ligand 1; fractalkine). They bind to S1PR, G2A, P2Y2 and CX3CR, respectively, on the phagocyte surface, promoting phagocyte migration to apoptotic cells.

In some embodiments, a platform cell described herein comprises a genomic disruption in at least one gene that inhibits phagocytosis of the cell. In some embodiments, the disruption is of a gene selected from the group consisting of CD47 and CD31.

### Immune Cell Recruitment

In some embodiments described herein, are provided platform cells that express and secrete an exogenous protein that binds to an immune cell and attracts the immune cell to the platform cell. In some embodiments, the cell comprises an exogenous nucleic acid that encodes a secretory agent that binds to an immune cell and attracts the immune cell, for instance innate or adaptive immune cell to the platform cell.

In some embodiments, the exogenous protein is a cytokine or chemokine. In some embodiments, the protein selected from the group consisting of SIP (sphingosine-1-phosphate), LPC (lysophosphatidylcholine), nucleotides (ATP or UTP) and CX3CL1 (CX3C motif chemokine ligand 1; fractalkine), CX3CL1, and ICAM3. IL-8/CXCL8 chemokines seem to be important for Neutrophil migration to CCL2, CXCL9, CXCL10 appear to recruit CTLs and monocytes.

### (3) Methods of Genetic Modification

Genetic modification of a platform cell or vaccine cell (e.g., knocking-in transgenes or knocking-out undesirable genes) can be achieved by any known genetic engineering techniques, for instance, but not restricted to endonucleases, including but are not limited to zinc-finger nucleases (ZFN), transcription activator-like effector nucleases (TALENs), and RNA-guided CRISPR-Cas9 nuclease (CRISPR/Cas9; Clustered Regular Interspaced Short Palindromic Repeats Associated 9).

### CRISPR System

The methods of making genetically engineered cells described herein can take advantage of a CRISPR system, including but not limited to knockout of NK cell inhibition ligands and knocking-in of NK cell activation ligands.

There are at least five types of CRISPR systems which all incorporate RNAs and Cas proteins. Types I, III, and IV assemble a multi-Cas protein complex that is capable of cleaving nucleic acids that are complementary to the crRNA. Types I and III both require pre-crRNA processing prior to assembling the processed crRNA into the multi-Cas protein complex. Types II and V CRISPR systems comprise a single Cas protein complexed with at least one guiding RNA.

The general mechanism and recent advances of CRISPR system are discussed in Cong, L. et al, “Multiplex genome engineering using CRISPR systems,” Science, 339(6121): 819-823 (2013); Fu, Y. et al., “High-frequency off-target mutagenesis induced by CRISPR-Cas nucleases in human cells,” Nature Biotechnology, 31, 822-826 (2013); Chu, V T et al. “Increasing the efficiency of homology-directed repair for CRISPR-Cas9-induced precise gene editing in mammalian cells,” Nature Biotechnology 33, 543-548 (2015); Shmakov, S. et al, “Discovery and functional characterization of diverse Class 2 CRISPR-Cas systems,” Molecular Cell, 60, 1-13 (2015); Makarova, K S et al, “An updated evolutionary classification of CRISPR-Cas systems,”, Nature Reviews Microbiology, 13, 1-15 (2015). Site-specific cleavage of a target DNA occurs at locations determined by both 1) base-pairing complementarity between the guide RNA and the target DNA (also called a protospacer) and 2) a short motif in the target DNA referred to as the protospacer adjacent motif (PAM). For example, an engineered cell can be generated using a CRISPR system, e.g., a type II CRISPR system. A Cas enzyme used in the methods disclosed herein can be Cas9, which catalyzes DNA cleavage. Enzymatic action by Cas9 derived from Streptococcus pyogenes or any closely related Cas9 can generate double stranded breaks at target site sequences which hybridize to 20 nucleotides of a guide sequence and that have a protospacer-adjacent motif (PAM) following the 20 nucleotides of the target sequence.

A CRISPR system can be introduced to a cell or to a population of cells using any means. In some embodiments, a CRISPR system may be introduced by electroporation or nucleofection. Electroporation can be performed for example, using the Neon® Transfection System (ThermoFisher Scientific) or the AMAXA® Nucleofector (AMAXA® Biosystems). Electroporation parameters may be adjusted to optimize transfection efficiency and/or cell viability. Electroporation devices can have multiple electrical wave form pulse settings such as exponential decay, time constant and square wave. Every cell type has a unique optimal Field Strength (E) that is dependent on the pulse parameters applied (e.g., voltage, capacitance and resistance). Application of optimal field strength causes electropermeabilization through induction of transmembrane voltage, which allows nucleic acids to pass through the cell membrane. In some embodiments, the electroporation pulse voltage, the electroporation pulse width, number of pulses, cell density, and tip type may be adjusted to optimize transfection efficiency and/or cell viability.

### Cas Protein

A vector can be operably linked to an enzyme-coding sequence encoding a CRISPR enzyme, such as a Cas protein (CRISPR-associated protein). In some embodiments, a nuclease or a polypeptide encoding a nuclease is from a CRISPR system (e.g., CRISPR enzyme). In some embodiments, the CRISPR enzyme directs cleavage of one or both strands at a target sequence. In some embodiments, the CRISPR enzyme mediates cleavage of both strands at a target DNA sequence (e.g., creates a double strand break in a target DNA sequence).

Non-limiting examples of Cas proteins can include Casl, CaslB, Cas2, Cas3, Cas4, Cas5, Cas6, Cas7, Cas8, Cas9 (also known as Csnl or Csxl2), CaslO, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, CsxlO, Csxl6, CsaX, Csx3, Csxl, CsxlS, Csfl, Csf2, CsO, Csf4, Cpf 1, c2c1, c2c3, Cas9HiFi, homologues thereof, or modified versions thereof. In some embodiments, a catalytically dead Cas protein can be used (e.g., catalytically dead Cas9 (dCas9)). An unmodified CRISPR enzyme can have DNA cleavage activity, such as Cas9. In some embodiments, a nuclease is Cas9. In some embodiments, a polypeptide encodes Cas9. In some embodiments, a nuclease or a polypeptide encoding a nuclease is catalytically dead. In some embodiments, a nuclease is a catalytically dead Cas9 (dCas9). In some embodiments, a polypeptide encodes a catalytically dead Cas9 (dCas9). A Cas protein can be a high fidelity Cas protein such as Cas9HiFi.

While S. pyogenes Cas9 (SpCas9) is commonly used as a CRISPR endonuclease for genome engineering, it may not be the best endonuclease for every target excision site. For example, the PAM sequence for SpCas9 (5′ NGG 3′) is abundant throughout the human genome, but an NGG sequence may not be positioned correctly to target a desired gene for modification. In some embodiments, a different endonuclease may be used to target certain genomic targets. In some embodiments, synthetic SpCas9-derived variants with non-NGG PAM sequences may be used. Additionally, other Cas9 orthologues from various species have been identified and these “non-SpCas9s” bind a variety of PAM sequences that can also be useful for the present disclosure. For example, the relatively large size of SpCas9 (approximately 4 kb coding sequence) means that plasmids carrying the SpCas9 cDNA may not be efficiently expressed in a cell. Conversely, the coding sequence for Staphylococcus aureus Cas9 (SaCas9) is approximately 1 kilo base shorter than SpCas9, possibly allowing it to be efficiently expressed in a cell. Similar to SpCas9, the SaCas9 endonuclease is capable of modifying target genes in mammalian cells in vitro and in mice in vivo.

Alternatives to S. pyogenes Cas9 may include RNA-guided endonucleases from the Cpf 1 family that display cleavage activity in mammalian cells. Unlike Cas9 nucleases, the result of Cpf1-mediated DNA cleavage is a double-strand break with a short 3′ overhang. Cpf1's staggered cleavage pattern may open up the possibility of directional gene transfer, analogous to traditional restriction enzyme cloning, which may increase the efficiency of gene editing. Like the Cas9 variants and orthologues described above, Cpf1 may also expand the number of sites that can be targeted by CRISPR to AT-rich regions or AT-rich genomes that lack the NGG PAM sites favored by SpCas9.

A vector that encodes a CRISPR enzyme comprising one or more nuclear localization sequences (NLSs), such as at least 1, 2, 3, 4, 5, 6, 7, 8, 9, 10, NLSs can be used. For example, a CRISPR enzyme can comprise at least 1, 2, 3, 4, 5, 6, 7, 8, 9, 10, NLSs at or near the ammo-terminus, at least 1, 2, 3, 4, 5, 6, 7, 8, 9, 10, NLSs at or near the carboxyl-terminus, or any combination of these (e.g., one or more NLS at the ammo-terminus and one or more NLS at the carboxyl terminus). When more than one NLS is present, each can be selected independently of others, such that a single NLS can be present in more than one copy and/or in combination with one or more other NLSs present in one or more copies. The NLS can be located anywhere within the polypeptide chain, e.g., near the N- or C-terminus. For example, the NLS can be within or within about 1, 2, 3, 4, 5, 10, 15, 20, 25, 30, 40, 50 amino acids along a polypeptide chain from the N- or C-terminus. Sometimes the NLS can be within or within about 50 amino acids or more, e.g., 100, 200, 300, 400, 500, 600, 700, 800, 900, or 1000 amino acids from the N- or C-terminus.

Any functional concentration of Cas protein can be introduced to a cell. For example, 15 micrograms of Cas mRNA can be introduced to a cell. In other cases, a Cas mRNA can be introduced from 0.5 micrograms to 100 micrograms. A Cas mRNA can be introduced from 0.5, 5, 10, 15, 20, 25, 30, 35, 40, 45, 50, 55, 60, 65, 70, 75, 80, 85, 90, 95, or 100 micrograms.

In some embodiments, a dual nickase approach may be used to introduce a double stranded break or a genomic break. Cas proteins can be mutated at known amino acids within either nuclease domains, thereby deleting activity of one nuclease domain and generating a nickase Cas protein capable of generating a single strand break. A nickase along with two distinct guide RNAs targeting opposite strands may be utilized to generate a double strand break (DSB) within a target site (often referred to as a “double nick” or “dual nickase” CRISPR system). This approach can increase target specificity because it is unlikely that two off-target nicks will be generated within close enough proximity to cause a DSB.

Guiding Polynucleic Acids (gRNA or gDNA)

A guiding polynucleic acid (or a guide polynucleic acid) can be DNA (gDNA) or RNA (gRNA). A guiding polynucleic acid can be single stranded or double stranded. In some embodiments, a guiding polynucleic acid can contain regions of single stranded areas and double stranded areas. A guiding polynucleic acid can also form secondary structures.

In some embodiments, the guide nucleic acid is a gRNA. In some embodiments, the gRNA comprises a guide sequence that specifies a target site and guides an RNA/Cas complex to a specified target DNA for cleavage. Site-specific cleavage of a target DNA occurs at locations determined by both 1) base-pairing complementarity between a gRNA and a target DNA (also called a protospacer) and 2) a short motif in a target DNA referred to as a protospacer adjacent motif (PAM). Similarly, a gRNA can be specific for a target DNA and can form a complex with a nuclease to direct its nucleic acid-cleaving activity.

In some embodiments, the gRNA comprises two RNAs, e.g., CRISPR RNA (crRNA) and transactivating crRNA (tracrRNA). In some embodiments, the gRNA comprises a single-guide RNA (sgRNA) formed by fusion of a portion (e.g., a functional portion) of crRNA and tracrRNA. In some embodiments, the gRNA comprises a dual RNA comprising a crRNA and a tracrRNA. In some embodiments, the gRNA comprises a crRNA and lacks a tracrRNA. In some embodiments, the crRNA hybridizes with a target DNA or protospacer sequence.

In some embodiments, the gRNA targets a nucleic acid sequence of or of about 20 nucleotides. In some embodiments, the gRNA targets a nucleic acid sequence of or of about 5, 10, 15, 16, 17, 18, 19, 20, 21, 22, 23, 24, 25, 30 or more nucleotides. In some embodiments, the gRNA binds a genomic region from about 1 base pair to about 20 base pairs away from a PAM. In some embodiments, the gRNA binds a genomic region from about 1, 2, 3, 4, 5, 6, 7, 8, 9, 10, 11, 12, 13, 14, 15, 16, 17, 18, 19, or up to about 20 base pairs away from a PAM. In some embodiments, the gRNA binds a genomic region within about 1, 2, 3, 4, 5, 6, 7, 8, 9, or 10 base pairs away from a PAM.

A guide RNA can also comprise a dsRNA duplex region that forms a secondary structure. For example, a secondary structure formed by a guide RNA can comprise a stem (or hairpin) and a loop. The length of a loop and a stem can vary. For example, a loop can range from about 3 to about 10 nucleotides in length, and a stem can range from about 6 to about 20 base pairs in length. A stem can comprise one or more bulges of 1 to about 10 nucleotides. The overall length of a second region can range from about 16 to about 60 nucleotides in length. For example, a loop can be or can be about 4 nucleotides in length and a stem can be or can be about 12 base pairs. A dsRNA duplex region can comprise a protein-binding segment that can form a complex with an RNA-binding protein, such as a RNA-guided endonuclease, e.g., Cas protein.

In some embodiments, a Cas protein, such as a Cas9 protein or any derivative thereof, is pre-complexed with a gRNA to form a ribonucleoprotein (RNP) complex. In some embodiments, the RNP complex is introduced into a cell to mediate editing.

In some embodiments, the gRNA is modified. The modifications can comprise chemical alterations, synthetic modifications, nucleotide additions, and/or nucleotide subtractions. The modifications can also enhance CRISPR genome engineering. A modification can alter chirality of a gRNA. In some embodiments, chirality may be uniform or stereopure after a modification. In some embodiments, the modification enhances stability of the gRNA.

In some embodiments, the modification is a chemical modification. A modification can be selected from 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′ deoxyribonucleoside analog purine, 2′ deoxyribonucleoside analog pyrimidine, ribonucleoside analog, 2′-O-methyl ribonucleoside analog, and sugar modified analogs, wobble/universal bases, fluorescent dye label, 2′ fluoro RNA, 2′ O-methyl RNA, methylphosphonate, phosphodiester DNA, phosphodiester RNA, phosphothioate DNA, phosphorothioate RNA, UNA, pseudouridine-5′-triphosphate, and 5-methylcytidine-5′-triphosphate, and any combination thereof.

In some embodiments, the modification comprise a phosphorothioate internucleotide linkage. In some embodiments, the gRNA comprises from 1 to 10, 1 to 5, or 1-3 phosphorothioate. In some embodiments, the gRNA comprises 1, 2, 3, 4, 5, 6, 7, 8, 9, 10, 11, 12, 13, 14, 15, 16, 17, 18, 19, or 20 phosphorothioate linkages. In some embodiments, the gRNA comprises phosphorothioate internucleotide linkages at the N terminus, C terminus, or both N terminus and C terminus. For example, in some embodiments, the gRNA comprises phosphorothioate internucleotide linkages between the N terminal 3-5 nucleotides, the C terminal 3-5 nucleotides, or both.

In some embodiments, the modification is a 2′-O-methyl phosphorothioate addition. In some embodiments, the gRNA comprises 1-10, 1-9, 1-8, 1-7, 1-6, 1-5, 1-4, 1-3, or 1-2 2′-O-methyl phosphorothioates. In some embodiments, the gRNA comprises from 1 to 10, 1 to 5, or 1-3 2′-O-methyl phosphorothioates. In some embodiments, the gRNA comprises 1, 2, 3, 4, 5, 6, 7, 8, 9, 10, 11, 12, 13, 14, 15, 16, 17, 18, 19, or 20 2′-O-methyl phosphorothioates. In some embodiments, the gRNA comprises 2′-O-methyl phosphorothioate internucleotide linkages at the N terminus, C terminus, or both N terminus and C terminus. For example, in some embodiments, the gRNA comprises 2′-O-methyl phosphorothioate internucleotide linkages between the N terminal 3-5 nucleotides, the C terminal 3-5 nucleotides, or both.

A gRNA can be introduced at any functional concentration. In some embodiments, 0.5 micrograms to 100 micrograms of the gRNA is introduced into a cell. In some embodiments, 0.5, 5, 10, 15, 20, 25, 30, 35, 40, 45, 50, 55, 60, 65, 70, 75, 80, 85, 90, 95, or 100 micrograms of the gRNA is introduced into a cell.

### Other Endonucleases

Other endonuclease based gene editing systems known in the art can be used to make an engineered cell described herein. For example, zinc finger nuclease systems and TALEN systems.

ZFNs are targeted nucleases comprising a nuclease fused to a zinc finger DNA binding domain. A “zinc finger DNA binding domain” or “ZFBD” is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but are not limited to, C2H2 zinc fingers, C3H zinc fingers, and C4 zinc fingers. A “designed” zinc finger domain is a domain not occurring in nature whose design/composition results principally from rational criteria, e.g., application of substitution rules and computerized algorithms for processing information in a database storing information of existing ZFN designs and binding data. A “selected” zinc finger domain is a domain not found in nature whose production results primarily from an empirical process such as phage display, interaction trap or hybrid selection. The most recognized example of a ZFN in the art is a fusion of the FokI nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-di-residues (RVD). The most recognized example of a TALEN in the art is a fusion polypeptide of the FokI nuclease to a TAL effector DNA binding domain.

Another example of a targeted nuclease that finds use in the methods described herein is a targeted Spoil nuclease, a polypeptide comprising a Spoil polypeptide having nuclease activity fused to a DNA binding domain, e.g., a zinc finger DNA binding domain, a TAL effector DNA binding domain, etc. that has specificity for a DNA sequence of interest. Additional examples of targeted nucleases suitable for the present invention include, but are not limited to Bxbl, phiC31, R4, PhiBTi, and WO/SPBc/TP901-1, whether used individually or in combination.

Any one of the aforementioned methods comprising genomically editing via use of an endonuclease can result in a genomic disruption. The genomic disruption can be sufficient to result in reduction or elimination of expression of the protein encoded by the gene. In some cases, a genomic disruption can also refer to the incorporation of an exogenous transgene into the cellular genome. In such cases, an exogenous transgene can also be detected. The genomic disruption can be detected in at least about 10%, 20%, 30%, 40%, 50%, 60%, 70%, 80%, 90% or 100% of cells tested. Detection can be performed by evaluating the disruption at the genomic level via sequencing, at the mRNA level, or protein level. Suitable methods include PCR, qPCR, flow cytometry, imaging, ELISA, NGS, and any combination thereof. In some cases, protein expression can be reduced by about 1 fold, 2 fold, 3 fold, 5 fold, 10 fold, 20 fold, 30 fold, 50 fold, 70 fold, 100 fold, 125 fold, 150 fold, 200 fold, 250 fold, 300 fold, 350 fold, 500 fold, or up to about 1000 fold as compared to a comparable method that lacks the use of the gene editing, such as with CRISPR.

### Transgenes

A transgene polynucleic acid encoding an exogenous protein or polypeptide that is knocked into a platform or vaccine cell described herein can be DNA or RNA, single-stranded or double stranded and can be introduced into a cell in linear or circular form. A transgene sequence(s) can be contained within a DNA minicircle, which may be introduced into the cell in circular or linear form. If introduced in linear form, the ends of a transgene sequence can be protected (e.g., from exonucleolytic degradation) by any method. For example, one or more dideoxynucleotide residues can be added to the 3′ terminus of a linear molecule and/or self-complementary oligonucleotides can be ligated to one or both ends. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A transgene can be flanked by recombination arms. In some instances, recombination arms can comprise complementary regions that target a transgene to a desired integration site. A transgene can also be integrated into a genomic region such that the insertion disrupts an endogenous gene. A transgene can be integrated by any method, e.g., non-recombination end joining and/or recombination directed repair. A transgene can also be integrated during a recombination event where a double strand break is repaired. A transgene can also be integrated with the use of a homologous recombination enhancer. For example, an enhancer can block non-homologous end joining so that homology directed repair is performed to repair a double strand break.

A transgene can be flanked by recombination arms where the degree of homology between the arm and its complementary sequence is sufficient to allow homologous recombination between the two. For example, the degree of homology between the arm and its complementary sequence can be 50% or greater. Two homologous non-identical sequences can be any length and their degree of non-homology can be as small as a single nucleotide (e.g., for correction of a genomic point mutation by targeted homologous recombination) or as large as 10 or more kilobases (e.g., for insertion of a gene at a predetermined ectopic site in a chromosome). Two polynucleotides comprising the homologous non-identical sequences need not be the same length.

A polynucleotide can be introduced into a cell as part of a vector molecule having additional sequences such as, for example, replication origins, promoters and genes encoding antibiotic resistance. Moreover, transgene polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)). A virus that can deliver a transgene can be an AAV virus.

A transgene is generally inserted so that its expression is driven by the endogenous promoter at the integration site, namely the promoter that drives expression of the endogenous gene into which a transgene is inserted. A transgene may comprise a promoter and/or enhancer, for example a constitutive promoter or an inducible or tissue/cell specific promoter. A minicircle vector can encode a transgene.

A transgene can be inserted into an endogenous gene such that all, some or none of the endogenous gene is expressed. For example, a transgene as described herein can be inserted into an endogenous locus such that some (N-terminal and/or C-terminal to a transgene) or none of the endogenous sequences are expressed, for example as a fusion with a transgene. In other cases, a transgene (e.g., with or without additional coding sequences such as for the endogenous gene) is integrated into any endogenous locus, for example a safe-harbor locus.

When endogenous sequences (endogenous or part of a transgene) are expressed with a transgene, the endogenous sequences can be full-length sequences (wild-type or mutant) or partial sequences. The endogenous sequences can be functional. Non-limiting examples of the function of these full length or partial sequences include increasing the serum half-life of the polypeptide expressed by a transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

### (4) Cell Compositions

Platform cells described herein can be stored long term for use in vaccine cells as appropriate. Specifically, these cells can be incorporated in appropriate compositions that are stable when frozen or cryopreserved. In some cases, provided are compositions for maintaining the pluripotency of engineered induced pluripotent stem cells (iPSCs) that are used as platform cells. In some cases, the composition comprises (i) engineered iPSC platform cells, and (ii) a pluripotency maintenance composition, for instance a small molecule composition comprising a MEK inhibitor, a GSK3 inhibitor and a ROCK inhibitor. In some embodiments, the platform cells are obtained from reprogramming engineered non-pluripotent cells, wherein the obtained iPSCs comprise the same targeted integration and/or in/del at selected sites in the engineered non-pluripotent cells. In some embodiments, the engineered iPSCs are obtained from engineering a clonal iPSC or a pool of iPSCs by introducing one or more targeted integration and/or in/del at one or more selected sites. In some other embodiments, the genome-engineered iPSCs are obtained from genome engineering by introducing one or more targeted integration and/or in/del at one or more selected sites to a pool of reprogramming non-pluripotent cells in contact with one or more reprogramming factors and optionally a small molecule composition comprising a TGFP receptor/ALK inhibitor, a MEK inhibitor, a GSK3 inhibitor and/or a ROCK inhibitor.

Engineered platform cells of the composition can comprise one or more exogenous polynucleotides encoding safety switch proteins, targeting modality, receptors, signaling molecules, transcription factors, pharmaceutically active proteins and peptides, drug target candidates, or proteins promoting engraftment, trafficking, homing, viability, self-renewal, persistence, and/or survival of the non-pluripotent cell reprogrammed iPSCs or derivative cells thereof; and/or in/dels at one or more endogenous genes associated with targeting modality, receptors, signaling molecules, transcription factors, drug target candidates, immune response regulation and modulation, or proteins suppressing engraftment, trafficking, homing, viability, self-renewal, persistence, and/or survival of the non-pluripotent cell reprogrammed iPSCs or derivative cells thereof.

In some embodiments, one or more exogenous polynucleotides encoding one or more exogenous polypeptides or proteins are operatively linked to (1) one or more exogenous promoters comprising CMV, EFla, PGK, CAQ UBC, or other constitutive, inducible, temporal-, tissue-, or cell type-specific promoters; or (2) one or more endogenous promoters comprised in selected sites in the platform cell comprising AAVS1, CCR5, ROSA26, collagen, HTRP, Hll, beta-2 microglobulin, GAPDH, TCR or RUNX1. In some embodiments, the composition further comprises one or more endonuclease capable of selected site recognition for introducing double strand break at selected sites.

### (5) Vaccine Cell Differentiation and Expansion

Vaccine cells described herein are made by further engineering the cells to comprise a nucleic acid encoding an exogenous microbial protein, or an antigenic fragment thereof, or express the exogenous microbial protein, or an antigenic fragment thereof. Thereby, the vaccine cells, when administered to a subject will induce an immune response specifically against the exogenous microbial protein.

Therefore, in one aspect, provided herein are genetically engineered cells that comprise i) a genomic disruption in at least one human leukocyte antigen (HLA) gene or at least one transcriptional regulator of an HLA gene; and ii) expression of an exogenous protein that binds to a phagocytic or cytolytic innate immune cell and stimulates activity (e.g., phagocytosis, cytolytic activity, proinflammatory cytokine secretion) of the innate immune cell; and iii) express an exogenous microbial protein or antigenic fragment thereof. In some embodiments, the vaccine cells comprise an antigen expression construct described herein.

The vaccine cells described herein can be any cell suitable for administration to a subject and delivery of a microbial protein. In some embodiments, the cells are differentiated from the platform cells. In some embodiments, the cells are differentiated from platform cells, wherein the platform cells are stem cells (e.g., iPSCs). In some embodiments, the cells are epithelial cells. In some embodiments, the cells are endothelial cells.

### Methods of Differentiation

In some embodiments, the vaccine cells are differentiated from the platform cells, wherein the platform cells are stem cells. In some embodiments, the stem cells are induced pluripotent stem cells. In some embodiments, the iPSCs are differentiated into epithelial cells or endothelial cells. In certain aspects, the iPSCs are differentiated into a cell type that has inherently low-immunogenicity to recipient T cells and allows the differentiated cells to be a focused target for NK cell-mediated vaccination. In certain additional aspects, the iPSCs are engineered to present kill-tags or suicide switch genes that can be activated to target the cell by administration of an antibody or small molecule.

Differentiation of pluripotent stem cells requires a change in the culture system, such as changing the stimuli agents in the culture medium or the physical state of the cells. The most conventional strategy utilizes the formation of embryoid bodies (EBs) as a common and critical intermediate to initiate the lineage-specific differentiation. “Embryoid bodies” are three-dimensional clusters that have been shown to mimic embryo development as they give rise to numerous lineages within their three-dimensional area. Through the differentiation process, typically a few hours to days, simple EBs (for example, aggregated pluripotent stem cells elicited to differentiate) continue maturation and develop into a cystic EB at which time, typically days to a few weeks, they are further processed to continue differentiation. EB formation is initiated by bringing pluripotent stem cells into close proximity with one another in three-dimensional multilayered clusters of cells, typically this is achieved by one of several methods including allowing pluripotent cells to sediment in liquid droplets, sedimenting cells into “U” bottomed well-plates or by mechanical agitation. To promote EB development, the pluripotent stem cell aggregates require further differentiation cues, as aggregates maintained in pluripotent culture maintenance medium do not form proper EBs. As such, the pluripotent stem cell aggregates need to be transferred to differentiation medium that provides eliciting cues towards the lineage of choice. EB-based culture of pluripotent stem cells typically results in generation of differentiated cell populations (ectoderm, mesoderm and endoderm germ layers) with modest proliferation within the EB cell cluster. Although proven to facilitate cell differentiation, EBs, however, give rise to heterogeneous cells in variable differentiation state because of the inconsistent exposure of the cells in the three-dimensional structure to differentiation cues from the environment. In addition, EBs are laborious to create and maintain. Moreover, cell differentiation through EB is accompanied with modest cell expansion, which also contributes to low differentiation efficiency.

The engineered iPSCs described herein can be differentiated using biomaterial scaffolds. Biomaterial scaffolds promote the viability and differentiation of stem cells seeded inside depending on the intrinsic properties of the material as well as the incorporation of specific chemical and physical cues into the material. Both natural and synthetic biomaterials can serve as the starting point for generating bioactive scaffolds for controlling stem cell differentiation into the desired tissue type. These scaffolds can take several different forms, which in turn have unique features. These scaffolds can also be combined to yield novel hybrid materials that certain formulations enable better cell survival. Suitable biomaterial scaffolds include but not limited to hydrogels, electrospun scaffolds, nano- and micro-particles using protein-based biomaterials (e.g., collagen, fibrin, silk, laminin, fibronectin and vitronectin), polysaccharide-based biomaterials (e.g., agarose, alginate, hyaluronan, chitosan, cellulose and its derivatives and decellularized extracellular matrix), synthetic biomaterials (e.g., poly (lactic-co-glycolic acid) (PLGA), poly (ethylene glycol) (PEG), poly caprolactone (PCL), polypyrrole (Ppy) and polydimethylsiloxane (PDMS)), and ceramic-based biomaterials.

In some cases, a method provided herein can yield reduced toxicity as compared to a comparable method. In some cases, toxicity can be reduced by about 1 fold, 2 fold, 3 fold, 4 fold, 5 fold, 6 fold, 7 fold, 8 fold, 9 fold, 10 fold, 11 fold, 15 fold, 20 fold, 25 fold, 50 fold, 100 fold, 300 fold, 500 fold, 800 fold, 1000 fold.

### Cell Types

In some cases, a method can comprise differentiating iPS cells. In some embodiments, a stem cell (e.g., iPSC) can be differentiated into an epithelial cell. In some cases, an iPS cell can be differentiated into a skin epithelial cell, lung epithelial cell, a gastrointestinal epithelial cell, a lung alveoli epithelial cell, mouth epithelial cell, vaginal epithelial cell, renal epithelial cell, renal tube epithelial cell, respiratory tract epithelial cell, bladder epithelial cell, urinary tract epithelial cell, blood vessel epithelial cell, brain epithelial cell, heart epithelial cell, ear epithelial cell, tongue epithelial cell, a cervical epithelial cell, a prostate epithelial cell, a breast epithelial cell, a uterus epithelial cell, tracheal epithelial cell, a large intestine epithelial cell, a small intestine epithelial cell, a colon epithelial cell, or a liver epithelial cell.

### APC Mimicry

In some cases, an iPS cell can be differentiated into an epithelial (dendritic) Antigen Presenting Cell (APC). By differentiating an iPS cell into a dendritic cell one can achieve APC mimicry thereby conferring upon a vaccine an MHC null and/or NK/Mo Innate Immunity+APC being presented to a subject's native MHC specific APC/innate immune system. This can result in a superior and/or safer immune antigenic response and/or naturalizing Ab production thereby conferring a lasting immunity post administration. In other cases, an iPS cell can be differentiated into a skin, lung, or GI/gut epithelial cell thus allowing for natural physiologic presentation of the immunogen to the host immune system. This may facilitate and/or enhance vaccine application for pulmonary application of the vaccine directly delivery to the lungs and/or p.o. per oral route of administration in addition to standard sub-cutaneous and intradermal and other skin and dermal based vaccine delivery methods.

### (6) Antigen Expression Constructs

Provided herein are antigen expression constructs and engineered cells comprising the constructs. In some embodiments, the antigen expression construct comprises a nucleic acid encoding an exogenous protein, or antigenic fragment thereof. In some embodiments, the exogenous protein comprises an exogenous antigenic protein. In some embodiments, the construct comprises a two or more exogenous proteins, or antigenic fragments thereof. In some embodiments, the nucleic acid is DNA or RNA. In some embodiments, the nucleic acid is cDNA. In some embodiments, the nucleic acid is mRNA. In some embodiments, the nucleic acid comprises a self-amplifying RNA. In some embodiments, the self amplifying RNA comprises a viral replicase. In some embodiments, the self-amplifying RNA comprises a subgenomic promoter region. In some embodiments, the self-amplifying RNA comprises a TAR. In some embodiments, the self-amplifying RNA comprises a sequence encoding an exogenous protein. The self-amplifying RNA promotes exponential replication of the exogenous protein.

In some embodiments, the exogenous protein is a microbial protein. In some embodiments, the microbial protein is a viral protein, a bacterial protein, a parasitic protein, or a protozoa protein.

In some embodiments, the cells or EVs provided herein are co-transfected with two or more nucleic acid constructs (e.g., a TAT-TAR system provided herein)

### COVID-19

In some embodiments, the microbial protein is a viral protein. In some embodiments, the viral protein is of a virus of order Nidovirales. In some embodiments, the viral protein is of a virus of family Coronaviridae. In some embodiments, the viral protein is of genus Alphacoronavirus, Betacoronavirus, Gammacoronavirus, and Deltacoronavirus. In some embodiments, the viral protein is of a virus of genus Betacoronavirus. In some embodiments, the viral protein is of a virus of subgenus Sarbecovirus. In some embodiments, the viral protein is of a virus of species severe acute respiratory syndrome-related coronavirus 2. In some embodiments, the viral protein is of a virus of strain severe acute respiratory syndrome coronavirus 2. In some embodiments, the viral protein is a spike protein of severe acute respiratory syndrome coronavirus 2.

In some embodiments, a cellular vaccine described herein is used to treat coronavirus disease 2019 (COVID-19). As a severe respiratory disease firstly reported in Wuhan, Hubei province, China, COVID-19 is also known as COVID-2019, 2019 novel coronavirus, or 2019-nCoV.

The COVID-19 disease is caused by the severe acute respiratory syndrome coronavirus 2 (SARS-CoV-2). SARS-CoV-2's genome has been sequenced and the order of genes (from 5′ to 3′) are as follows: replicase ORF1ab, spike (S), envelope (E), membrane (M) and nucleocapsid (N). Wu, F., Zhao, S., Yu, B., Chen, Y., Wang, W., Song, Z., Hu, Y., Tao, Z., Tian, J. Pei, Y., Yuan, M., Zhang, Y., Dai, F., Liu, Y., Wang, Q., Zheng, J., Xu, L., Holmes, E., & Zhang, Y., A new coronavirus associated with human respiratory disease in China. Nature 579, 265-269 (2020) (the entire contents of which is incorporated by reference herein for all purposes).

SARS-CoV-2 makes use of a densely glycosylated spike protein (S protein) to gain entry into host cells. The coronavirus spike protein is a trimeric class I fusion protein that exists in a metastable prefusion conformation that undergoes a substantial structural rearrangement to fuse the viral membrane with the host cell membrane. This process is triggered when the S1 subunit binds to a host cell receptor. Receptor binding destabilizes the prefusion trimer, resulting in shedding of the S1 subunit and transition of the S2 subunit to a stable post-fusion conformation. To engage a host cell receptor, the receptor-binding domain (RBD) of S1 undergoes hinge-like conformational movements that transiently hide or expose the determinants of receptor binding. These two states are referred to as the “down” conformation and the “up” conformation, where down corresponds to the receptor-inaccessible state and up corresponds to the receptor-accessible state, which is thought to be less stable. Because of the indispensable function of the S protein, it represents a target for antibody-mediated neutralization, and characterization of the prefusion S structure would provide atomic-level information to guide vaccine design and development. Wrapp, D., Wang, N., Corbett, K., Goldsmith, J., Hsieh, C., Abiona, O., Graham, B. & McLellan, J., Cryo-EM structure of the 2019-nCoV spike in the prefusion conformation, SCIENCE, 13 Mar. 2020: 1260-1263 (the entire contents of which is incorporated by reference herein for all purposes).

The amino acid sequence of the SARS-CoV-2 S protein is (obtained from NCBI):

>YP_009724390.1 surface glycoprotein
[Severe acute respiratory syndrome coronavirus 2]
(SEQ ID NO: 1)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGENCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT.

The SARS-CoV-2's spike protein is composed of about 1,273 amino acids and contain several domains. Wu et al., Wrapp et al., and Xia, S., Zhu, Y., Liu, M., Lan, Q., Xu, W., Wu, Y., Ying, T., Liu, S., Shi, Z., Jiang, S. & Lu, L., Fusion mechanism of 2019-nCoV and fusion inhibitors targeting HR1 domain in spike protein. Cell Mol Immunol (2020). https://doi.org/10.1038/s41423-020-0374-2. The spike protein contains: signal sequence (SS); N-terminal domain (NTD, 14-305 aa); receptor-binding domain (RBD, 319-541 aa); S1/S2 protease cleavage site (S1/S2, R685/S686); fusion peptide (FP, 788-806 aa from Zhu et al. or 816-833 aa from Wrapp et al.); heptad repeat 1 (HR1, 912-984 aa); central helix (CH, 986-1035 aa from Wrapp et al.); connector domain (CD, 1076-1141 aa from Wrapp et al.); heptad repeat 2 (HR2, 1163-1213 aa); transmembrane domain (TM, 1214-1237 aa); and cytoplasmic tail (CT, 1238-1273 aa). The NTD sequence is: SEQ ID NO: 2. The RBD sequence is: SEQ ID NO: 3. The FB sequence is: SEQ ID NO: 4 or SEQ ID NO: 5. The HR1 sequence is: SEQ ID NO: 6. The CH sequence is: SEQ ID NO: 7. The CD sequence is: SEQ ID NO: 8. The HR2 sequence is: SEQ ID NO: 9. The TM sequence is: SEQ ID NO: 10. The CT sequence is: SEQ ID NO: 11.

In some embodiments, the antigen expression construct comprises a nucleic acid sequence encoding a protein with at least 50%, 60%, 70%, 75%, 80%, 85%, 90%, 91%, 92%, 93%, 94%, 95%, 96%, 97%, 98%, 99%, or 100% identity to SEQ ID NO: 1, 2, 3, 4, 5, 6, 7, 8, 9, 10, or 11. In some embodiments, the antigen expression construct comprises a nucleic acid sequence encoding a protein with at least 50%, 60%, 70%, 75%, 80%, 85%, 90%, 91%, 92%, 93%, 94%, 95%, 96%, 97%, 98%, 99%, or 100% identity to SEQ ID NO: 1, 2, 3, 4, 5, 6, 7, 8, 9, 10, or 11, or an antigen fragment thereof that is at least 10, 15, 20, 25, 30, 40, 50, or 100 amino acid acids in length.

A phylogenetic and genetic comparison analysis of the S gene and its regions showed minor variations among strains. The RBD sequences of WHCV (WH-Human 1 coronavirus: the SARS-CoV-2 strain identified in Wu et al.) were more closely related to those of SARS-CoVs (73.8-74.9% amino acid identity) and SARS-like CoVs, including strains Rs4874, Rs7327 and Rs4231 (75.9-76.9% amino acid identity), that are able to use the human ACE2 receptor for cell entry. In addition, the RBD of the spike protein from WHCV was only one amino acid longer than the RBD of the spike protein from SARS-CoV. The previously determined crystal structure of the RBD of the spike protein of SARS-CoV complexed with human ACE2 (Protein Data Bank (PDB) 2AJF) revealed that regions 433-437 and 460-472 directly interact with human ACE2 and hence may be important in determining species specificity. Thus, the S protein is a primary target for the development of effective vaccines against SARS-CoV-2. In some embodiments, the inventors of the present application develop a cellular vaccine against SARS-CoV-2 using a living cell transfected with a construct containing SARS-CoV-2 S protein.

The nucleic acid sequence encoding the SARS-CoV-2 S protein is: NCBI reference number NC_045512.2:21563-25384 encoding the Severe acute respiratory syndrome coronavirus 2 isolate Wuhan-Hu-1, complete genome, Gene ID: 43740568 provided herein as SEQ ID NO: 12. When incorporating the nucleic acid sequence into the construct, the above sequence can be codon-optimized.

In some cases, a nucleic acid sequence to be incorporated into a construct provided herein may be modified. Modifications can comprise truncations of a sequence. For example, modifications can comprise deletion of the cytoplasmic tail, deletion of the transmembrane domain, deletion of a furin cleavage site, and any combination thereof. In some embodiments, the nucleic acid comprises a 5′ cap. In some embodiments, the nucleic acid comprises a 5′-untranslated region. In some embodiments, the nucleic acid comprises a 3′ poly-A tail. Modifications can also include additions of a sequence. Additions can comprise a trimerization tag, transgene sequences, or both. In some cases, modifications can also comprise mutations, for example a sequence encoding a proline mutation. Any number of modifications can be introduced such as 1, 2, 3, 4, 5, 6, 7, 8, 9, or up to about 10 mutations.

In some embodiments, the cells or EVs provided herein are co-transfected with an ARRDC1-TAT plasmid and a self-amplifying TAR-SARS-CoV2 spike RNA sequence.

### Other Pathogens

Any antigen or combination of antigens can be employed in connection with the embodiments disclosed herein by engineering the UVC platform to provide a polypathogenic and polyvalent vaccine cell. In some embodiments, the antigen expression construct comprises a nucleic acid encoding a microbial protein (or antigenic fragment thereof) of a rabies virus, Ebola virus, HIV, influenza virus, avian influenza virus, SARS coronavirus, herpes virus, Caliciviruses, hepatitis viruses, Zika virus, West Nile virus, La Crosse encephalitis, California encephalitis, Venezuelan equine encephalitis, Eastern equine encephalitis, Western equine encephalitis, Japanese encephalitis virus, St. Louis encephalitis virus, Yellow fever virus, Chikungunya virus or norovirus.

In some cases, an influenza antigen peptide may be utilized. In influenza viral antigen may be human or non-human. In some cases, an influenza viral antigen that is used originates from type A, B, C, and/or D. In some cases, an influenza viral antigen is A(H1N1), A(H3N2), B(Victoria), or B(Yamagata). In some cases, an influenza viral antigen can be from a non-human species, such as swine, bird, bat, bovine, canine, horse, poultry, feline, and the like.

In some embodiments, the antigen expression construct comprises a nucleic acid encoding a microbial protein (or antigenic fragment thereof) from a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (EBOV) (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus (CMV)), Chikungunya, Hantavirus, Hypoviridae, Iridoviridae, Leviviridae, Lipothrixviridae, Microviridae, Orthomyxoviridae (e.g., Influenza virus A, such as H1N1 strain, and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3. Viral antigens may be derived from a particular strain such as a papilloma virus, a herpes virus, i.e., herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, JC virus, West Nile Virus, cytomeglavirus, Epstein-Barr, rotavirus, rhinovirus, adenovirus, coxsackieviruses, equine encephalitis, BK virus, malaria, MuLV, VSV, HTLV, Japanese encephalitis, yellow fever, Rift Valley fever, and lymphocytic choriomeningitis.

In some cases, an antigen can be from a coronavirus and is SARS-CoV-2, SARS-CoV, and/or MERS-CoV or any variant thereof. In some cases, a viral peptide can be from a variant of SARS-CoV-2. In some cases, a variant of SARS-CoV-2 can comprise B.1.1.7 (or the U.K. variant), B.1.1.207, Cluster 5, B.1.351 (or RSA variant), P.1 (or Brazil variant), B.1.617 (or India variant), B.1525, NS3, WIV04/2019, or CAL.20C. In some embodiments, the B.1.617 variant includes a mutation in a spike protein comprising at least one of E154K, E484Q, L452R, P681R, Q1071H, or any combination thereof. In some embodiments, a variant of SARS-CoV-2 comprises lineage A.1, A.2, A.3, A.4, A.5, A.6, B.1, B.2, B.3, B.4, B.5, B.6, B.7, B.8, B.9, B.10, B.11, B.12, B.13, B.14, B.15, or B.16.

In some embodiments, the antigen expression construct comprises a nucleic acid encoding a microbial protein (or antigenic fragment thereof) from a virus of one or more of Influenza virus A, Influenza virus B, Influenza virus C, Isavirus, Thogotovirus and Quaranjavirus. Exemplary influenza A virus subtypes include H1N1, H1N2, H3N2, H3N1, H5N1, H2N2, and H7N7. Exemplary influenza virus antigens include one or more proteins or glycoproteins such as hemagglutinin, such as HA1 and HA2 subunits, neuraminidase, viral RNA polymerase, such as one or more of PB1, PB2 PA and PB1-F2, reverse transcriptase, capsid protein, non-structured proteins, such as NS 1 and NEP, nucleoprotein, matrix proteins, such as M1 and M2 and pore proteins. In some embodiments, Influenza A virus antigens include one or more of the Hemagglutinin (HA) or Neuraminidase (NA) glycoproteins or fragments of the HA or NA, including the antigenic sites of the Hemagglutinin HA1 glycoprotein. In an exemplary embodiment, MDNPs include RNA encoding the influenza A/WSN/33 HA protein.

In some embodiments, the antigen expression construct comprises a nucleic acid encoding a microbial protein (or antigenic fragment thereof) from a virus of one or more of Ebolavirus, for example, the Zaire ebolavirus (EBOV), Sudan ebolavirus (SUDV), Tai Forest ebolavirus (TAFV), Reston ebolavirus (RESTV), and Bundibugyo ebolavirus (BDBV). In an exemplary embodiment, MDNPs include RNA, such as repRNA, encoding the Zaire ebolavirus glycoprotein (GP), or one or more fragments of the Zaire ebolavirus glycoprotein (GP).

In some embodiments, the antigen expression construct comprises a nucleic acid encoding a microbial protein (or antigenic fragment thereof) from a virus of one or more of the genus Flavivirus, for example, the Zika virus (ZIKV).

In some cases, more than one nucleic acids are expressed in a cell vaccine. For example, at least 2, at least 3, or at least 4 can be comprised in a cellular vaccine. In some cases, at least 2 are expressed by a cellular vaccine and are from SARS-CoV-2 and influenza (H1N1).

In some embodiments, the antigen expression construct comprises a nucleic acid encoding a microbial protein (or antigenic fragment thereof) of Bacillus anthracis, Clostridium botulinum, Yersinia pestis, Variola major, Francisella tularensis, poxviridae, Burkholderia pseudomallei, Coxiella burnetiid, Brucella species, Burkholderia mallei, Chlamydia psittaci, Staphylococcus enterotoxin B, Diarrheagenic E. coli, Pathogenic Vibrios, Shigella species, Salmonella, Listeria monocytogenes, Campylobacter jejuni, Yersinia enterocolitica, Acinetobacter baumannii, Pseudomonas aeruginosa, Enterobacteriaceae, Enterococcus faecium, Staphylococcus aureus, Helicobacter pyloni, Campylobacter spp., Salmonellae, Neisseria gonorrhoeae, Streptococcus pneumoniae, Haemophilus influenzae or Shigella spp.

In some embodiments, the antigen expression construct comprises a nucleic acid encoding a microbial protein (or antigenic fragment thereof) of Cryptosporidium parvum, Cyclospora cayatanensis, Giardia lamblia, Entamoeba histolytica, Toxoplasma gondii, Naegleria fowleri or Balamuthia mandrillaris.

In some embodiments, a peptide or fragment thereof from another pathogen to be utilized in a vaccine can have from about 50%, 60%, 70%, 75%, 80%, 85%, 88%, 90%, 93%, 95%, 96%, 97%, 98%, 99%, or 10000 identity to any sequence from Table 5.

TABLE 5
Exemplary viral peptides and corresponding MHC alleles.

| SEQ | | | | | | MHC |
|---|---|---|---|---|---|---|
| ID | | | IEDB | | | Allele |
| NO: | | Protein | ID | Peptide | MHC Allele | Class |
| 63 | HIV 1 | Gag | 59613 | SLYNTVATL | HLA-A*02:01/ | I |
| | | | | | HLA-A*02:02/ | |
| | | | | | HLA-A*02:03/ | |
| | | | | | HLA-A*02:06/ | |
| | | | | | HLA-A*02:11/ | |
| | | | | | HLA-A*02:19/ | |
| | | | | | HLA-A2/ | |
| | | | | | HLA-B*15:01/ | |
| | | | | | HLA-A*02:05/ | |
| | | | | | HLA-A*02:14/ | |
| | | | | | HLA-A*68:02/ | |
| | | | | | HLA-A*69:01/ | |
| | | | | | HLA-B*07:02 | |
| 13 | HIV 1 | Gag | 33250 | KRWIILGLNK | HLA-B*27:05/ | I |
| | | | | | HLA-B*27:03/ | |
| | | | | | HLA-A*03:01/ | |
| | | | | | HLA-B*27:02/ | |
| | | | | | HLA-DRB1*01:01/ | |
| | | | | | Mamu-B*017:04/ | |
| | | | | | HLA-DRB1*01:03 | |
| 14 | HIV 1 | Gag | 21635 | GPGHKARVL | HLA-B*07:02/ | I |
| | | | | | H2-Dd/ | |
| | | | | | HLA-A*01:01/ | |
| | | | | | HLA-A*02:01/ | |
| | | | | | HLA-A*03:01/ | |
| | | | | | HLA-A*11:01/ | |
| | | | | | HLA-A*31:01/ | |
| | | | | | HLA-A*69:01/ | |
| | | | | | HLA-B*15:01/ | |
| | | | | | HLA-B*27:05/ | |
| | | | | | HLA-B*40:01/ | |
| | | | | | HLA-B*58:01 | |
| 15 | HIV 1 | Gag | 29804 | KAFSPEVIPMF | HLA-B*57:01/ | I |
| | | | | | HLA-B*57:03 | |
| 16 | HIV 1 | Gag | 69360 | VLAEAMSQV | HLA-A*02:01/ | I |
| | | | | | HLA-A*02:02/ | |
| | | | | | HLA-A*02:03/ | |
| | | | | | HLA-A*02:06/ | |
| | | | | | HLA-A*68:02/ | |
| | | | | | HLA-A*02:11/ | |
| | | | | | HLA-A*02:19/ | |
| | | | | | HLA-A*69:01 | |
| 17 | HIV 1 | Gag | 131070 | SLFNTVATL | HLA-A*02:01 | I |
| 18 | HIV 1 | Nef | 56620 | RYPLTFGWCF | HLA-A*24:02 | I |
| 19 | HIV 1 | Nef | 5295 | AVDLSHFLK | HLA-A*11:01/ | I |
| | | | | | HLA-A*03:01/ | |
| | | | | | HLA-A*01:01/ | |
| | | | | | HLA-A*02:01/ | |
| | | | | | HLA-A*24:02 | |
| 20 | HIV 1 | Nef | 52760 | QVPLRPMTYK | HLA-A*03:01/ | I |
| | | | | | HLA-A*11:01/ | |
| | | | | | HLA-A*02:01/ | |
| | | | | | HLA-A*31:01/ | |
| | | | | | HLA-A*33:01/ | |
| | | | | | HLA-A*68:01/ | |
| | | | | | HLA-A*01:01/ | |
| | | | | | HLA-A*02:02/ | |
| | | | | | HLA-A*02:05/ | |
| | | | | | HLA-A*24:02 | |
| 21 | HIV 1 | Nef | 102046 | RYPLTFGW | HLA-A*24:02 | I |
| 22 | HIV 1 | Nef | 193060 | RFPLTFGWCF | HLA-A*24:02 | I |
| 23 | HIV 1 | Envelope | 53935 | RGPGRAFVTI | H2-Dd/H2-Db/ | I |
| | | glycoprotein | | | H2-Ld/H2-Kb/ | |
| | | gp160 | | | H2-Kd/ | |
| | | | | | HLA-A*02:01/ | |
| | | | | | HLA-A*02:01/ | |
| | | | | | HLA-A2/ | |
| | | | | | HLA-A*02:02/ | |
| | | | | | HLA-A*02:03/ | |
| | | | | | HLA-A*02:06/ | |
| | | | | | HLA-A*68:02 | |
| 24 | HIV 1 | Env gp160 | 32201 | KLTPLCVTL | HLA-A*02:01/ | I |
| | | | | | HLA-A*02:02/ | |
| | | | | | HLA-A*02:03/ | |
| | | | | | HLA-A*02:06/ | |
| | | | | | HLA-A*02:11/ | |
| | | | | | HLA-A*02:19/ | |
| | | | | | HLA-A*68:02/ | |
| | | | | | HLA-A*69:01 | |
| 25 | HIV 1 | Env gp160 | 54226 | RIQRGPGRAFVTIGK | HLA-DQA1*03:01/ | II |
| | | | | | DQB1*03:02 | |
| 26 | HIV 1 | Env gp160 | 53114 | RAIEAQQHL | HLA-C*12:02/ | I |
| | | | | | HLA-B*40:01/ | |
| | | | | | HLA-A*31:01/ | |
| | | | | | HLA-A*69:01/ | |
| | | | | | HLA-B*07:02/ | |
| | | | | | HLA-B*15:01/ | |
| | | | | | HLA-B*58:01/ | |
| | | | | | HLA-C*03:01/ | |
| | | | | | HLA-A*01:01/ | |
| | | | | | HLA-A*02:01/ | |
| | | | | | HLA-A*03:01/ | |
| | | | | | HLA-A*11:01/ | |
| | | | | | HLA-B*27:05 | |
| 27 | HIV 1 | Env gp160 | 54226 | RIQRGPGRAFVTIGK | HLA-DQA1*03:01/ | II |
| | | | | | DQB1*03:02 | |
| 28 | HIV 1 | Env gp160 | 67245 | TVYYGVPVWK | HLA-A*11:01/ | I |
| | | | | | HLA-A*03:01/ | |
| | | | | | HLA-A*31:01/ | |
| | | | | | HLA-A*68:01/ | |
| | | | | | HLA-A*33:01 | |
| 29 | HIV 1 | Env gp160 | 54730 | RLRDLLLIVTR | HLA-A*11:01/ | I |
| | | | | | HLA-A*03:01/ | |
| | | | | | HLA-A*01:01/ | |
| | | | | | HLA-A*02:01/ | |
| | | | | | HLA-A*24:02/ | |
| | | | | | HLA-A*33:03/ | |
| | | | | | HLA-A*07:02 | |
| 30 | Zaire | Nucleoprotein | 16888 | FLSFASLFL | HLA-A*02:01/ | I |
| | ebola virus | | | | HLA-A*24:02/ | |
| | | | | | HLA-B*15:01/ | |
| | | | | | HLA-C*03:03/ | |
| | | | | | HLA-A*03:01/ | |
| | | | | | HLA-A*25:01/ | |
| | | | | | HLA-A*26:01/ | |
| | | | | | HLA-A*80:01/ | |
| | | | | | HLA-A*18:01/ | |
| | | | | | HLA-B*18:01/ | |
| | | | | | HLA-B*27:03/ | |
| | | | | | HLA-B*46:01/ | |
| | | | | | HLA-B*57:01 | |
| 31 | Zaire | Nucleoprotein | 17527 | FQQTNAMVT | HLA-A*02:01/ | I |
| | ebola virus | | | | HLA-B*15:01 | |
| 32 | Zaire | Nucleoprotein | 32188 | KLTEAITAA | HLA-A*02:02/ | I |
| | ebola virus | | | | HLA-B*15:01 | |
| 33 | Zaire | Nucleoprotein | 54673 | RLMRTNFLI | HLA-A*02:01/ | I |
| | ebola virus | | | | HLA-A*24:02/ | |
| | | | | | HLA-A*03:01/ | |
| | | | | | HLA-A*11:01/ | |
| | | | | | HLA-A*08:01/ | |
| | | | | | HLA-B*15:01/ | |
| | | | | | HLA-B*07:02/ | |
| | | | | | HLA-A*25:01/ | |
| | | | | | HLA-A*26:01/ | |
| | | | | | HLA-B*18:01/ | |
| | | | | | HLA-B*46:01 | |
| 34 | Zaire | Nucleoprotein | 75566 | YQNNLEEI | HLA-A*24:02/ | I |
| | ebola virus | | | | HLA-A*02:01/ | |
| | | | | | HLA-B*15:01 | |
| 35 | Zaire | Envelope | 91144 | ATDVPSATK | HLA-A*11:01/ | I |
| | ebola virus | glycoprotein | | | HLA-A*01:01/ | |
| | | | | | HLA-A*03:01/ | |
| | | | | | HLA-A*24:02 | |
| 36 | Zaire | Envelope | 54480 | RLASTVIYR | HLA-A*03:01/ | I |
| | ebola virus | glycoprotein | | | HLA-A*11:01/ | |
| | | | | | HLA-A*02:01/ | |
| | | | | | HLA-A*31:01/ | |
| | | | | | HLA-A*02:03/ | |
| | | | | | HLA-A*02:12/ | |
| | | | | | HLA-A*02:19/ | |
| | | | | | HLA-A*23:01/ | |
| | | | | | HLA-A*24:03/ | |
| | | | | | HLA-A*25:01/ | |
| | | | | | HLA-A*26:01/ | |
| | | | | | HLA-A*68:02/ | |
| | | | | | HLA-A*69:01/ | |
| | | | | | HLA-A*80:01/ | |
| | | | | | HLA-B*15:01/ | |
| | | | | | HLA-B*15:17/ | |
| | | | | | HLA-B*18:01/ | |
| | | | | | HLA-B*27:03/ | |
| | | | | | HLA-B*39:01/ | |
| | | | | | HLA-B*46:01/ | |
| | | | | | HLA-B*51:01/ | |
| | | | | | HLA-B*57:01 | |
| 37 | Zaire | Envelope | 66646 | TTIGEWAFW | HLA-A*24:02/ | I |
| | ebola virus | glycoprotein | | | HLA-A*32:07/ | |
| | | | | | HLA-A*32:15/ | |
| | | | | | HLA-A*68:23/ | |
| | | | | | HLA-B*15:42/ | |
| | | | | | HLA-B*45:06/ | |
| | | | | | HLA-B*58:01/ | |
| | | | | | HLA-B*83:01/ | |
| | | | | | HLA-C*04:01/ | |
| | | | | | HLA-A*02:01/ | |
| | | | | | HLA-A*02:03/ | |
| | | | | | HLA-A*02:11/ | |
| | | | | | HLA-A*02:12/ | |
| | | | | | HLA-A*02:16/ | |
| | | | | | HLA-A*02:19/ | |
| | | | | | HLA-A*03:01/ | |
| | | | | | HLA-A*26:01/ | |
| | | | | | HLA-A*68:02/ | |
| | | | | | HLA-A*69:01/ | |
| | | | | | HLA-A*80:01/ | |
| | | | | | HLA-B*15:01/ | |
| | | | | | HLA-B*18:01/ | |
| | | | | | HLA-B*27:03/ | |
| | | | | | HLA-B*39:01/ | |
| | | | | | HLA-B*46:01 | |
| 38 | Zaire | Envelope | 91362 | GFRSGVPPK | HLA-A*03:01/ | I |
| | ebola virus | glycoprotein | | | HLA-A*11:01/ | |
| | | | | | HLA-B*15:01 | |
| 39 | Zaire | Envelope | 91766 | NQDGLICGL | HLA-A*02:01 | I |
| | ebola virus | glycoprotein | | | | |
| 40 | Zika Virus | Genome | 569587 | IGVSNRDFV | H2-Db/H2-Kb | I |
| | | polyprotein | | | | |
| 41 | Zika Virus | Genome | 741567 | IRCIGVSNRDFVEGMSGGTW | HLA-DRB1*01:01/ | II |
| | | polyprotein | | | HLA-DTB1*03:01// | |
| | | | | | HLA-DTB1*04:01/ | |
| | | | | | HLA-DTB1*07:01/ | |
| | | | | | HLA-DTB1*15:01/ | |
| | | | | | HLA-DTB5*01:01/ | |
| | | | | | HLA-DTB1*11:01 | |
| 42 | Zika Virus | Genome | 741871 | QPENLEYRIMLSVHGSQHSG | HLA-DRB5*01:01/ | II |
| | | polyprotein | | | HLA-DRB1*03:01/ | |
| | | | | | HLA-DRB1*04:01/ | |
| | | | | | HLA-DRB1*07:01/ | |
| | | | | | HLA-DRB1*11:01/ | |
| | | | | | HLA-DRB1*15:01/ | |
| | | | | | HLA-DRB5*01:01 | |
| 43 | Zika Virus | Genome | 741599 | KGVSYSLCTAAFTFTKIPAE | HLA-DRB1*01:01/ | II |
| | | polyprotein | | | HLA-DRB1*04:01/ | |
| | | | | | HLA-DRB1*07:01/ | |
| | | | | | HLA-DRB1*11:01/ | |
| | | | | | HLA-DRB1*15:01/ | |
| | | | | | HLA-DRB5*01:01/ | |
| | | | | | HLA-DRB1*03:01 | |
| 44 | Zika Virus | Genome | 741402 | FEATVRGAKRMAVLGDTAWD | HLA-DRB1*01:01/ | II |
| | | polyprotein | | | HLA-DRB1*03:01/ | |
| | | | | | HLA-DRB1*07:01/ | |
| | | | | | HLA-DRB1*11:01/ | |
| | | | | | HLA-DRB1*15:01/ | |
| | | | | | HLA-DRB5*01:01// | |
| | | | | | HLA-DRB1*04:01 | |
| 45 | Zika Virus | Genome | 741533 | HRSGSTIGKAFEATVRGAKR | HLA-DRB1*01:01/ | II |
| | | polyprotein | | | HLA-DRB1*04:01/ | |
| | | | | | HLA-DRB1*11:01/ | |
| | | | | | HLA-DRB1*15:01/ | |
| | | | | | HLA-DRB5*01:01/ | |
| | | | | | HLA-DRB1*03:01/ | |
| | | | | | HLA-DRB1*07:01 | |
| 46 | Influenza A | Hemagglutinin | 125913 | ELLVLLENERTLDYHDS | HLA-DRB1*04:01 | II |
| | (H1N1, 2009) | | | | | |
| 47 | Influenza A | Hemagglutinin | 125913 | ELLVLLENERTLDYHDS | HLA-DRB1*04:01 | II |
| 48 | Influenza A | Matrix | 67496 | TYVLSIIPSGPLKAEIAQRL | HLA-DRB1*04:01 | II |
| | | protein 1 | | | | |
| 49 | Influenza A | Matrix | 124495 | LYKKLKREITF | HLA-A*24:02 | I |
| | | protein 1 | | | | |
| 50 | Influenza A | Neuraminidase | 126100 | GFEMIWDPNGWTGTDN | HLA-DRB1*04:01 | II |
| 51 | Influenza A | Neuraminidase | 126167 | GQASYKIFRIEKGKIVK | HLA-DRB1*04:01 | II |
| 52 | Influenza A | Neuraminidase | 126199 | GWAIYSKDNSVRIGSKG | HLA-DRB1*04:01 | II |

### Cancer Peptides

In some embodiments, the antigen expression construct comprises a nucleic acid encoding a peptide (or a fragment thereof) associated with a cancer or a tumor. In some embodiments, the nucleic acid encodes a full-length protein or a fragment or derivative thereof. Exemplary peptides can be neoantigens or oncoproteins. In some embodiments, the peptide or fragment thereof comprises at least one of 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, b-Catenin, bcr-abl, bcr-abl p190 (e1a2), bcr-abl p210 (b2a2), bcr-abl p210 (b3a2), BING-4, CAG-3, CAIX, CAMEL, CISH, Caspase-8, CD171, CD19, CD20, CD22, CD23, CD24, CD30, CD33, CD38, CD44v7/8, CDC27, CDK-4, CEA, CLCA2, Cyp-B, DAM-10, DAM-6, DEK-CAN, EGFRvIII, EGP-2, EGP-40, ELF2, Ep-CAM, EphA2, EphA3, erb-B2, erb-B3, erb-B4, ES-ESO-1a, ETV6/AML, FBP, fetal acetylcholine receptor, FGF-5, FN, G250, GAGE-1, GAGE-2, GAGE-3, GAGE-4, GAGE-5, GAGE-6, GAGE-7B, GAGE-8, GD2, GD3, GnT-V, Gp100, gp75, Her-2, HLA-A*0201-R170I, HMW-MAA, HSP70-2 M, HST-2 (FGF6), HST-2/neu, hTERT, iCE, IL-11Ra, IL-13Ru2, KDR, KIAA0205, K-RAS, L1-cell adhesion molecule, LAGE-1, LDLR/FUT, Lewis Y, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, κ-light chain, or any combination thereof.

In some embodiments, a peptide comprises a neoantigen peptide. For example, a neoantigen can be a peptide that arises from polypeptide generated from genomic sequence that comprises an E805G mutation in ERBB2IP. Neoantigen and neoepitopes can be identified by whole-exome sequencing. In some cases, a gene that can comprise a mutation that gives rise to a neoantigen or neoepitope peptide can be ABL1, ACOl 1997, ACVR2A, AFP, AKT1, ALK, ALPPL2, ANAPC1, APC, ARID1A, AR, AR-v7, ASCL2, β2M, BRAF, BTK, C150RF40, CDH1, CLDN6, CNOT1, CT45A5, CTAG1B, DCT, DKK4, EEF1B2, EEF1DP3, EGFR, EIF2B3, env, EPHB2, ERBB3, ESR1, ESRP1, FAM11 IB, FGFR3, FRG1B, GAGE1, GAGE 10, GATA3, GBP3, HER2, IDH1, JAK1, KIT, KRAS, LMAN1, MABEB 16, MAGEA1, MAGEA10, MAGEA4, MAGEA8, MAGEB 17, MAGEB4, MAGEC1, MEK, MLANA, MLL2, MMP13, MSH3, MSH6, MYC, NDUFC2, NRAS, NY-ESO, PAGE2, PAGE5, PDGFRa, PIK3CA, PMEL, pol protein, POLE, PTEN, RAC1, RBM27, RNF43, RPL22, RUNX1, SEC31A, SEC63, SF3B 1, SLC35F5, SLC45A2, SMAP1, SMAP1, SPOP, TFAM, TGFBR2, THAP5, TP53, TTK, TYR, UBR5, VHL, XPOT.

In some embodiments, the peptide(s) or fragment(s) thereof are derived from a polypeptide, a polypeptide generated from a nucleic acid sequence, or a neoantigen derived from at least one of A1CF, ABI1, ABL1, ABL2, ACKR3, ACSL3, ACSL6, ACVR1, ACVR1B, ACVR2A, AFDN, AFF1, AFF3, AFF4, AKAP9, AKT1, AKT2, AKT3, ALDH2, ALK, AMER1, ANK1, APC, APOBEC3B, AR, ARAF, ARHGAP26, ARHGAP5, ARHGEF10, ARHGEF10L, ARHGEF12, ARID1A, ARID1B, ARID2, ARNT, ASPSCR1, ASXL1, ASXL2, ATF1, ATIC, ATM, ATP1A1, ATP2B3, ATR, ATRX, AXIN1, AXIN2, B2M, BAP1, BARD1, BAX, BAZ1A, BCL10, BCL11A, BCL11B, BCL2, BCL2L12, BCL3, BCL6, BCL7A, BCL9, BCL9L, BCLAF1, BCOR, BCORL1, BCR, BIRC3, BIRC6, BLM, BMP5, BMPR1A, BRAF, BRCA1, BRCA2, BRD3, BRD4, BRIP1, BTG1, BTK, BUB1B, C15orf65, CACNA1D, CALR, CAMTA1, CANT1, CARD11, CARS, CASP3, CASP8, CASP9, CBFA2T3, CBFB, CBL, CBLB, CBLC, CCDC6, CCNB1IP1, CCNC, CCND1, CCND2, CCND3, CCNE1, CCR4, CCR7, CD209, CD274, CD28, CD74, CD79A, CD79B, CDC73, CDH1, CDH10, CDH11, CDH17, CDK12, CDK4, CDK6, CDKN1A, CDKN1B, CDKN2A, CDKN2C, CDX2, CEBPA, CEP89, HCHD7, CHD2, CHD4, CHEK2, CHIC2, CHST11, CIC, CIITA, CLIP1, CLP1, CLTC, CLTCL1, CNBD1, CNBP, CNOT3, CNTNAP2, CNTRL, COL1A1, COL2A1, COL3A1, COX6C, CPEB3, CREB1, CREB3L1, CREB3L2, CREBBP, CRLF2, CRNKL1, CRTC1, CRTC3, CSF1R, CSF3R, CSMD3, CTCF, CTNNA2, CTNNB1, CTNND1, CTNND2, CUL3, CUX1, CXCR4, CYLD, CYP2C8, CYSLTR2, DAXX, DCAF12L2, DCC, DCTN1, DDB2, DDIT3, DDR2, DDX10, DDX3X, DDX5, DDX6, DEK, DGCR8, DICER1, DNAJB1, DNM2, DNMT1, DNMT3A, DROSHA, EBF1, ECT2L, EED, EGFR, EIF1AX, EIF3E, EIF4A2, ELF3, ELF4, ELK4, ELL, ELN, EML4, EP300, EPAS1, EPHA3, EPHA7, EPS15, ERBB2, ERBB3, ERBB4, ERC1, ERCC2, ERCC3, ERCC4, ERG, ESR1, ETNK1, ETV1, ETV4, ETV5, ETV6, EWSR1, EXT1, EXT2, EZH2, EZR, FAM131B, FAM135B, FAM46C, FAM47C, FANCA, FANCC, FANCD2, FANCE, FANCF, FANCG, FAS, FAT1, FAT3, FAT4, FBLN2, FBXO11, FBXW7, FCGR2B, FCRL4, FEN1, FES, FEV, FGFR1, FGFR1OP, FGFR2, FGFR3, FGFR4, FH, FHIT, FIP1L1, FKBP9, FLCN, FLI1, FLNA, FLT3, FLT4, FNBP1, FOXA1, FOXL2, FOXO1, FOXO3, FOXO4, FOXP1, FOXR1, FSTL3, FUBP1, FUS, GAS7, GATA1, GATA2, GATA3, GLI1, GMPS, GNA11, GNAQ, GNAS, GOLGA5, GOPC, GPC3, GPC5, GPHN, GRIN2A, GRM3, H3F3A, H3F3B, HERPUD1, HEY1, HIF1A, HIP1, HIST1H3B, HIST1H4I, HLA-A, HLF, HMGA1, HMGA2, HNF1A, HNRNPA2B1, HOOK3, HOXA11, HOXA13, HOXA9, HOXC11, HOXC13, HOXD11, HOXD13, HRAS, HSP90AA1, HSP90AB1, ID3, IDH1, IDH2, IGF2BP2, IKBKB, IKZF1, IL2, IL21R, IL6ST, IL7R, IRF4, IRS4, ISX, ITGAV, ITK, JAK1, JAK2, JAK3, JAZF1, JUN, KAT6A, KAT6B, KAT7, KCNJ5, KDM5A, KDM5C, KDM6A, KDR, KDSR, KEAP1, KIAA1549, KIF5B, KIT, KLF4, KLF6, KLK2, KMT2A, KMT2C, KMT2D, KNL1, KNSTRN, KRAS, KTN1, LARP4B, LASP1, LCK, LCP1, LEF1, LEPROTL1, LHFPL6, LIFR, LMNA, LMO1, LMO2, LPP, LRIG3, LRP1B, LSM14A, LYL1, LZTR1, MAF, MAFB, MALT1, MAML2, MAP2K1, MAP2K2, MAP2K4, MAP3K1, MAP3K13, MAPK1, MAX, MB21D2, MDM2, MDM4, MDS2, MECOM, MED12, MEN1, MET, MGMT, MITF, MKL1, MLF1, MLH1, MLLT1, MLLT10, MLLT11, MLLT3, MLLT6, MN1, MNX1, MPL, MSH2, MSH6, MSI2, MSN, MTCP1, MTOR, MUC1, MUC16, MUC4, MUTYH, MYB, MYC, MYCL, MYCN, MYD88, MYH11, MYH9, MYO5A, MYOD1, N4BP2, NAB2, NACA, NBEA, NBN, NCKIPSD, NCOA1, NCOA2, NCOA4, NCOR1, NCOR2, NDRG1, NF1, NF2, NFATC2, NFE2L2, NFIB, NFKB2, NFKBIE, NIN, NKX2-1, NONO, NOTCH1, NOTCH2, NPM1, NR4A3, NRAS, NRG1, NSD1, NSD2, NSD3, NT5C2, NTHL1, NTRK1, NTRK3, NUMA1, NUP214, NUP98, NUTM1, NUTM2A, NUTM2B, OLIG2, OMD, P2RY8, PABPC1, PAFAH1B2, PALB2, PATZ1, PAX3, PAX5, PAX7, PAX8, PBRM1, PBX1, PCBP1, PCM1, PD-1, PDCD1LG2, PDGFB, PDGFRA, PDGFRB, PDL1, PER1, PHF6, PHOX2B, PICALM, PIK3CA, PIK3CB, PIK3R1, PIM1, PLAG1, PLCG1, PML, PMS1, PMS2, POLD1, POLE, POLG, POT1, POU2AF1, POU5F1, PPARG, PPFIBP1, PPM1D, PPP2R1A, PPP6C, PRCC, PRDM1, PRDM16, PRDM2, PREX2, PRF1, PRKACA, PRKAR1A, PRKCB, PRPF40B, PRRX1, PSIP1, PTCH1, PTEN, PTK6, PTPN11, PTPN13, PTPN6, PTPRB, PTPRC, PTPRD, PTPRK, PTPRT, PWWP2A, QKI, RABEP1, RAC1, RAD17, RAD21, RAD51B, RAF1, RALGDS, RANBP2, RAP1GDS1, RARA, RB1, RBM10, RBM15, RECQL4, REL, RET, RFWD3, RGPD3, RGS7, RHOA, RHOH, RMI2, RNF213, RNF43, ROBO2, ROS1, RPL10, RPL22, RPL5, RPN1, RSPO2, RSPO3, RUNX1, RUNX1T1, S100A7, SALL4, SBDS, SDC4, SDHA, SDHAF2, SDHB, SDHC, SDHD, SEPT5, SEPT6, SEPT9, SET, SETBP1, SETD1B, SETD2, SF3B1, SFPQ, SFRP4, SGK1, SH2B3, SH3GL1, SHTN1, SIRPA, SIX1, SIX2, SKI, SLC34A2, SLC45A3, SMAD2, SMAD3, SMAD4, SMARCA4, SMARCB1, SMARCD1, SMARCE1, SMC1A, SMO, SND1, SNX29, SOCS1, SOX2, SOX21, SOX9, SPECC1, SPEN, SPOP, SRC, SRGAP3, SRSF2, SRSF3, SS18, SS18L1, SSX1, SSX2, SSX4, STAG1, STAG2, STAT3, STAT5B, STAT6, STIL, STK11, STRN, SUFU, SUZ12 SYK, TAF15, TAL1, TAL2, TBL1XR1, TBX3, TCEA1, TCF12, TCF3, TCF7L2, TCL1A, TEC, TERT, TET1, TET2, TFE3, TFEB, TFG, TFPT, TFRC, TGFBR2, THRAP3, TLX1, TLX3, TMEM127, TMPRSS2, TNC, TNFAIP3, TNFRSF14, TNFRSF17, TOP1, TP53, TP63, TPM3, TPM4, TPR, TRAF7, TRIM24, TRIM27, TRIM33, TRIP11, TRRAP, TSC1, TSC2, TSHR, U2AF1, UBR5, USP44, USP6, USP8, VAV1, VHL, VTI1A, WAS, WDCP, WIF1, WNK2, WRN, WT1, WWTR1, XPA, XPC, XPO1, YWHAE, ZBTB16, ZCCHC8, ZEB1, ZFHX3, ZMYM2, ZMYM3, ZNF331, ZNF384, ZNF429, ZNF479, ZNF521, ZNRF3, ZRSR2, or any combination thereof.

### Methods of Delivery

The antigen expression constructs described herein can be delivered to a target cell by any suitable means, e.g., any potentially be stably integrated into the cell genome at designated sites via genome engineering, such as safe harbor sites, for constitutive and predictable expression. An antigen expression construct can be targeted into a preferred genomic location. In some cases, an antigen expression construct can be stably integrated into a cellular genome. In some cases, an antigen expression construct is integrated into a safe harbor site, MHC locus, TCR locus, HLA locus, inhibitory receptor locus, and any combination thereof. Non-limiting examples of safe harbors can include HPRT, AAVS SITE (e.g., AAVS1, AAVS2, ETC.), CCR5, or Rosa26. In some cases, an antigen expression construct is transiently expressed. Conventional viral and non-viral based gene transfer methods can be used to introduce nucleic acids into cells. Methods of non-viral delivery of nucleic acids include electroporation, lipofection, nucleofection, gold nanoparticle delivery, microinjection, biolistics, virosomes, liposomes, immunoliposomes, polycation or lipid-nucleic acid conjugates, naked DNA, mRNA, artificial virions, and agent-enhanced uptake of DNA. Sonoporation using, e.g., the Sonitron 2000 system (Rich-Mar), can also be used for delivery of nucleic acids.

Non-viral vector delivery systems can include DNA plasmids, naked nucleic acids, nucleic acids complexed with a delivery vehicle such as a liposome or poloxamer, and delivery of an mRNA.

In one embodiment, the antigen expression construct is electroporated into the cell. In some embodiments, the antigen expression construct comprises mRNA and the mRNA is electroporated into the cell. Additional exemplary nucleic acid delivery systems include those provided by AMAXA Biosystems (Cologne, Germany), Life Technologies (Frederick, Md.), MAXCYTE, Inc. (Rockville, Md.), BTX Molecular Delivery Systems (Holliston, Mass.) and Copernicus Therapeutics Inc. (see for example U.S. Pat. No. 6,008,336). Lipofection reagents are sold commercially (e.g., TRANSFECTAM® and LIPOFECTIN®). Delivery can be to cells (ex vivo administration) or target tissues (in vivo administration).

The pathogen protein encoding polynucleotides and compositions comprising the polynucleotides described herein can be delivered using vectors containing sequences encoding one or more of the proteins. Any vector systems can be used including but not limited to plasmid vectors, retroviral vectors, lentiviral vectors, adenovirus vectors, poxvirus vectors, herpesvirus vectors and adeno-associated virus vectors, etc. Viral vector delivery systems can include DNA and RNA viruses, which have either episomal or integrated genomes after delivery to the cell.

### Suicide Gene

In some cases, a cell provided herein can comprise a genomic integration of a “kill-switch” suicide gene. A suicide gene can allow for removal of the cell by treatment with a drug that selectively kills those cells comprising the suicide gene. Inclusion of a suicide gene in a cell can also allow for increased safety when utilizing cells provided herein for treatment.

In some cases, a suicide gene may be incorporated into a cellular product. A suicide gene allows for the elimination of gene modified cells in the case of an adverse event, self-reactivity of infused cells, eradication of infection, and the like. In some embodiments, the suicide gene is introduced to a random genomic position, or a targeted locus (e.g., a metabolic gene locus, DNA/RNA replication gene locus, safe harbor, MHC locus, HLA locus, TCR locus, exhaustion locus, inhibitory receptor locus (PD-1, CTLA-4, Tim 3, CISH, and the like). Non-limiting examples of safe harbors can include HPRT, AAVS SITE (e.g., AAVS1, AAVS2, ETC.), CCR5, or Rosa26. In some cases, a suicide gene may be driven by an exogenous promoter or take advantage of an endogenous promoter of an integrated locus.

Various suicide genes are known in the art and can be utilized in the cellular compositions provided herein. Exemplary suicide genes can be: thymidine kinase/Ganciclovir, cytosine deaminase/5-fluorocytosine, nitroreductase/CB1954, carboxypeptidase G2/nitrogen mustard, cytochrome P450/oxazaphosphorine, purine nucleoside phosphorylase/6-methylpurine deoxyriboside (PNP/MEP), (HRP/IAA), and combinations thereof. In a specific embodiment, a suicide gene is an inducible caspase-9 gene (see US Pre-Grant Patent Publication No. US 2013/0071414, which suicide genes are incorporated by reference herein). Other suicide genes include a gene that encodes any one or more of: a conformationally intact binding epitope for pharmaceutical-grade anti-EGFR monoclonal antibody, cetuximab (Erbitux); EGFRt, a caspase polypeptide (e.g., iCasp9; Straathof et al., Blood 105:4247-4254, 2005; Di Stasi et al., N. Engl. ./. Med. 365: 1673-1683, 2011; Zhou and Brenner, Exp. Hematol. pii: S0301-472X (16)30513-6. doi: 10.1016/j.exphem.2016.07.011), RQR8 (Philip et al., Blood 124: 1277-1287, 2014), a 10-amino acid tag of the human c-myc protein (Myc) (Kieback et al., Proc. Natl. Acad. Sci. USA 105:623-628, 2008), as discussed herein, and a marker/safety switch polypeptide, such as RQR (CD20+CD34; Philip et al., 2014). In some embodiments, the suicide gene is sr39TK, which allows elimination of cells by the introduction of ganciclovir. This gene may also be used to image gene modified cells using positron emission tomography to localized cells in the recipient/host. A suicide gene may also be a chemically induced caspase, dimerization induced by a small molecule/chemically induced dimerizer (CID). The suicide gene may also be a selectable surface marker (CD 19 or CD20 or CD34 or EGFR or LNGFR, etc.) allowing the cells to be eliminated by introduction of an antibody through antibody dependent cellular cytotoxicity, complement cascade, etc.

In some cases, a suicide gene can be included within a vector comprising a viral antigen peptide provided herein. In other cases, a suicide gene is separately introduced into a cell, using for example a CRISPR system, a viral system, electroporation, transfection, transduction, and any combination thereof. In some cases, a suicide gene is knocked into a targeted locus.

### (7) Methods of Vaccinating a Subject

In one aspect, provided herein are methods of immunizing a subject against a pathogen by administering a population of vaccine cells described herein tailored to induce an adaptive immune response against the pathogen in the subject (e.g., the vaccine cells comprise a protein or antigen fragment of the pathogen.

In some embodiments, the pathogen is a virus, bacteria, or parasite.

In some embodiments, the pathogen is a virus. In some embodiments, the virus is a rabies virus, Ebola virus, HIV, influenza virus, avian influenza virus, SARS coronavirus, herpes virus, Caliciviruses, hepatitis viruses, zika virus, West Nile virus, La Crosse encephalitis, California encephalitis, Venezuelan equine encephalitis, Eastern equine encephalitis, Western equine encephalitis, Japanese encephalitis virus, St. Louis encephalitis virus, Yellow fever virus, Chikungunya virus, or norovirus.

In some embodiments, the virus is of order Nidovirales. In some embodiments, the virus is of family Coronaviridae. In some embodiments, the virus is of genus Alphacoronavirus, Betacoronavirus, Gammacoronavirus, and Deltacoronavirus. In some embodiments, the virus is of genus Betacoronavirus. In some embodiments, the virus is of subgenus Sarbecovirus. In some embodiments, the virus is of species severe acute respiratory syndrome-related coronavirus 2. In some embodiments, the virus is of strain severe acute respiratory syndrome coronavirus 2. In some embodiments, the virus is of severe acute respiratory syndrome coronavirus 2.

In some embodiments, the pathogen is a bacteria. In some embodiments, the bacteria is Bacillus anthracis, Clostridium botulinum, Yersinia pestis, Variola major, Francisella tularensis, poxviridae, Burkholderia pseudomallei, Coxiella burnetiid, Brucella species, Burkholderia mallei, Chlamydia psittaci, Staphylococcus enterotoxin B, Diarrheagenic E. coli, Pathogenic Vibrios, Shigella species, Salmonella, Listeria monocytogenes, Campylobacter jejuni, Yersinia enterocolitica, Acinetobacter baumannii, Pseudomonas aeruginosa, Enterobacteriaceae, Enterococcus faecium, Staphylococcus aureus, Helicobacter pylon, Campylobacter spp., Salmonellae, Neisseria gonorrhoeae, Streptococcus pneumoniae, Haemophilus influenzae or Shigella spp.

In some embodiments, the pathogen is a parasite. In some embodiments, the parasite is Cryptosporidium parvum, Cyclospora cayatanensis, Giardia lamblia, Entamoeba histolytica, Toxoplasma gondii, Naegleria fowleri or Balamuthia mandrillaris.

The cellular vaccine described herein may be administered by any suitable delivery route well known in the art, include but not limited to intramuscular injection, intradermal injection, intravenous injection or subcutaneous injection. In some embodiments, the vaccine is administered locally. In some embodiments, the vaccine is administered systemically. In some embodiments, the vaccine is administered using a pen-injector device, such as is used for at-home delivery of epinephrine, could be used to allow self-administration of the vaccine. In some cases, a vaccine is administered via post intradermal/SQ injection.

In some embodiments, a vaccine is administered locally. In some embodiments, the vaccine is administered subcutaneously. In some embodiments, the vaccine is self-administered by a patient.

In some cases, a vaccine is administered via a pulmonary system. In some cases, a vaccine is inhaled. In some cases, the vaccine is administered via inhalation. In some cases, the vaccine is inhaled and is able to access the lungs. In some cases, the vaccine is inhaled and able to access the airways. In some cases, the vaccine is administered orally. In some cases, the vaccine is administered orally and is able to access the gastrointestinal tract. In some cases a vaccine may be ingested orally via the GI system. In some cases, a vaccine is applied to the skin. In some cases the vaccine is administered through the skin. In some embodiments, the vaccine is administered via subcutaneous injection. In some embodiments, the vaccine is administered via dermal injection. In some embodiments, the vaccine is administered via intradermal injection. The use of such delivery devices may be particularly amenable to large scale immunization campaigns such as would be required during a pandemic.

### (8) Kits

Any of the compositions described herein may be comprised in a kit. In a non-limiting example, a vaccine may be in a kit, any type of cells may be provided in the kit, and/or reagents for manipulation of vaccines and/or cells may be provided in the kit. The components are provided in suitable container means.

The kits may comprise a suitably aliquoted composition. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits also will typically include a means for containing the components in close confinement for commercial sale. Such containers may include injection or blow-molded plastic containers into which the desired vials are retained.

However, the components of the kit may be provided as dried powder(s). When reagents and/or components are provided as a dry powder, the powder can be reconstituted by the addition of a suitable solvent. It is envisioned that the solvent may also be provided in another container means.

### Exemplary Embodiments

Some embodiments provide a genetically engineered human cell comprising (a) a genomic disruption in at least one human leukocyte antigen (HLA) gene or at least one transcriptional regulator of an HLA gene; and (b) an exogenous nucleic acid encoding a cell surface protein that binds to a protein expressed on the surface of a phagocytic or cytolytic immune cell, or a functional fragment or functional variant of the cell surface protein, wherein the binding results in the activation of phagocytic or cytolytic activity of the immune cell.

In some embodiments, the genomic disruption inhibits expression of an HLA protein encoded by the at least one HLA gene on the surface of the genetically engineered human cell. In some embodiments, the genomic disruption results in a reduction of HLA or MHC mediated T cell activation and/or proliferation as compared to a comparable cell lacking the genomic disruption. In some embodiments, the genomic disruption results in less HLA or MHC mediated T cell activation and/or proliferation as compared to a comparable cell lacking the genomic disruption. In some embodiments, the comparable cell comprises a human cell lacking the genomic disruption. In some embodiments, the comparable cell comprises a human cell expressing the HLA gene. In some embodiments, the comparable cell comprises the genetically engineered human cell lacking the disruption.

In some embodiments, the genomic disruption completely inhibits expression of an HLA protein encoded by the at least one HLA gene on the surface of the genetically engineered human cell.

In some embodiments, the genomic disruption in at least one human leukocyte antigen (HLA) gene or at least one transcriptional regulator of an HLA gene results in a reduction of HLA or MHC mediated T cell activation or proliferation upon administration of the genetically engineered human cells to a subject as compared to administration of comparable cells without the genomic disruption in at least one human leukocyte antigen (HLA) gene or at least one transcriptional regulator of an HLA gene. In some embodiments, the genomic disruption in at least one human leukocyte antigen (HLA) gene or at least one transcriptional regulator of an HLA gene results in a reduction of HLA or MHC mediated T cell activation or proliferation as compared to comparable cells without the genomic disruption in at least one human leukocyte antigen (HLA) gene or at least one transcriptional regulator of an HLA gene.

In some embodiments, the genomic disruption is in an HLA class I gene. In some embodiments, the HLA class I gene is an HLA-A gene, HLA-B gene, HLA-C gene, or (3-microglobulin gene. In some embodiments, the HLA class I gene is a β-microglobulin gene.

In some embodiments, the genomic disruption is in an HLA class II gene. In some embodiments, the HLA class II gene is an HLA-DP gene, HLA-DM gene, HLA-DOA gene, HLA-DOB gene, HLA-DQ gene, HLA-DR gene.

In some embodiments, at least one transcriptional regulator of the HLA gene is a CIITA gene, RFX5 gene, RFXAP gene, or RFXANK gene. In some embodiments, the HLA gene is a CIITA gene.

In some embodiments, the genetically engineered human cell comprises a genomic disruption in at least one HLA class I gene or the at least one transcriptional regulator of the HLA class I gene and a genomic disruption in at least one HLA class II gene or the at least one transcriptional regulator of the HLA class II gene.

In some embodiments, the genetically engineered human cell comprises a genomic disruption in at least one HLA class I transcriptional regulator gene and a genomic disruption in at least one HLA class II transcriptional regulator.

In some embodiments, the immune cell is an innate immune cell. In some embodiments, the innate immune cell is an NK cell, a macrophage, a dendritic cell, a neutrophil, or an eosinophil. In some embodiments, the innate immune cell is an NK cell.

In some embodiments, the binding results in the activation of cytolytic activity of the NK cell.

In some embodiments, the cell surface protein is a ligand that specifically binds to a natural killer (NK) cell activating receptor expressed on the surface of an NK cell. In some embodiments, the cell surface protein is selected from the group consisting of MICA, MICB, ULBP1, ULBP2, ULBP3, ULBP4, ULBP5, ULBP6, CD155, CD112 (Nectin-2), B7-H6, Necl-2, and immunoglobulin Fc.

In some embodiments, the cell surface protein is a natural killer (NK) cell activating ligand. In some embodiments, the natural killer cell activating ligand is selected from the group consisting of MICA, MICB, ULBP1, ULBP2, ULBP3, ULBP4, ULBP5, ULBP6, CD155, CD112 (Nectin-2), B7-H6, and Necl-2.

In some embodiments, the cell comprises an exogenous nucleic acid encoding a secretory protein that binds to a receptor expressed on the surface of a phagocytic or cytolytic immune cell, or a functional fragment or functional variant of the secretory protein, wherein the protein attracts the immune cell towards the genetically engineered human cell.

In some embodiments, the genetically engineered human cell comprises a nucleic acid encoding an exogenous protein, an antigenic fragment thereof, or a suicide gene. In some embodiments, the exogenous protein comprises a microbial protein

In some embodiments, the microbial protein comprises a nucleocapsid phosphoprotein comprising at least about 85% sequence identity to SEQ ID NO: 54.

In some embodiments, the microbial protein is secreted by the genetically engineered human cell, expressed on the surface of the genetically engineered human cell, or expressed within the cytoplasm of the genetically engineered human cell.

In some embodiments, the microbial protein is a viral, bacterial, parasitic, or protozoa protein. In some embodiments, the microbial protein is a viral protein. In some embodiments, the viral protein is of a virus of order Nidovirales. In some embodiments, the viral protein is of a virus of family Coronaviridae. In some embodiments, the viral protein is of a virus of subfamily Orthocoronavirinae. In some embodiments, the viral protein is of a virus of genus Alphacoronavirus, Betacoronavirus, Gammacoronavirus, and Deltacoronavirus. In some embodiments, the viral protein is of a virus of genus Betacoronavirus. In some embodiments, the viral protein is of a virus of subgenus Sarbecovirus. In some embodiments, the viral protein is of a virus of species severe acute respiratory syndrome-related coronavirus 2. In some embodiments, the viral protein is of a virus of strain severe acute respiratory syndrome coronavirus 2. In some embodiments, the viral protein is a spike protein of severe acute respiratory syndrome coronavirus 2. In some embodiments, the viral protein is a spike protein of SEQ ID NO: 1. In some embodiments, the viral protein is a spike protein encoded by SEQ ID NO: 53.

In some embodiments, the viral protein is of a virus selected from a group that comprises: influenza, Epstein-Barr virus (EBV), mega virus, Norwalk virus, coxsackie virus, middle east respiratory syndrome-related coronavirus, severe acute respiratory syndrome-related coronavirus, SARS-Cov-2 virus, hepatitis B, varicella zoster virus, parvovirus, adenovirus, Marburg virus, Ebola virus, Rabies, Smallpox, HIV, Hantavirus, Dengue, Rotavirus, MERS-CoV, mumps virus, cytomegalovirus (CMV), Herpes virus, papillomavirus, chikungunya virus, or any combination thereof.

In some embodiments, the genetically engineered human cell is differentiated from a stem cell. In some embodiments, the stem cell is an induced pluripotent stem cell (iPSC), an embryonic stem cell (ESC), an adult stem cell (ASC), a pluripotent stem cell (PSC), or a hematopoietic stem and progenitor cell (HSPC). In some embodiments, the stem cell is an induced pluripotent stem cell (iPSC).

In some embodiments, the genetically engineered human cell is an epithelial cell or endothelial cell. In some embodiments, the genetically engineered human cell is not a cancer cell.

In some embodiments, the genetically engineered human cell has been irradiated.

In some embodiments, the genetically engineered human cell is a stem cell.

In some embodiments, the stem cell is an induced pluripotent stem cell (iPSC), an embryonic stem cell (ESC), an adult stem cell (ASC), a pluripotent stem cell (PSC), or a hematopoietic stem and progenitor cell (HSPC). In some embodiments, the stem cell is an induced pluripotent stem cell (iPSC). In some embodiments, the genetically engineered human cell is incapable of proliferation in vitro, in vivo, or both.

In some embodiments, the genetically engineered human cell is for use in a vaccine.

In some embodiments, the at least one genomic disruption is mediated by an endonuclease. In some embodiments, the endonuclease is a CRISPR endonuclease, a Zinc finger nuclease (ZFN), or a Transcription Activator-Like Effector Nuclease (TALEN).

In some embodiments, the at least one genomic disruption is mediated by a CRISPR system that comprises an endonuclease and a guide RNA (gRNA), wherein the gRNA comprises an RNA sequence complementary to a DNA sequence of the at least one HLA gene or at least one transcriptional regulator of the HLA gene.

Some embodiments provide a genetically engineered human cell comprising: (a) a genomic disruption in at least one human leukocyte antigen (HLA) gene or at least one transcriptional regulator of an HLA gene; (b) a nucleic acid encoding an exogenous cell surface protein that binds to a protein expressed on the surface of a phagocytic or cytolytic immune cell, or a functional fragment or functional variant of the exogenous cell surface protein, wherein the binding results in the activation of phagocytic or cytolytic activity of the immune cell; and (c) a nucleic acid encoding an exogenous antigenic protein, or an antigenic fragment thereof.

In some embodiments, the exogenous antigenic protein, or antigenic fragment thereof, is a microbial protein, or an antigenic fragment thereof. In some embodiments, the exogenous antigenic protein comprises a nucleocapsid phosphoprotein comprising at least about 85% sequence identity to SEQ ID NO: 54.

In some embodiments, the microbial protein is secreted by the genetically engineered human cell, expressed on the surface of the genetically engineered human cell, or expressed within the cytoplasm of the genetically engineered human cell.

In some embodiments, the microbial protein is a viral, bacterial, parasitic, or protozoa protein. In some embodiments, the microbial protein is a viral protein. In some embodiments, the viral protein is of a virus of order Nidovirales. In some embodiments, the viral protein is of a virus of family Coronaviridae. In some embodiments, the viral protein is of a virus of subfamily Orthocoronavirinae. In some embodiments, the viral protein is of a virus of genus Alphacoronavirus, Betacoronavirus, Gammacoronavirus, and Deltacoronavirus. In some embodiments, the viral protein is of a virus of genus Betacoronavirus. In some embodiments, the viral protein is of a virus of subgenus Sarbecovirus. In some embodiments, the viral protein is of a virus of species severe acute respiratory syndrome-related coronavirus 2. In some embodiments, the viral protein is of a virus of strain severe acute respiratory syndrome coronavirus 2. In some embodiments, the viral protein is a spike protein of severe acute respiratory syndrome coronavirus 2. In some embodiments, the viral protein is a spike protein of SEQ ID NO: 1. In some embodiments, the viral protein is a spike protein encoded by SEQ ID NO: 53.

In some embodiments, the viral protein is from a virus selected from a group that comprises: influenza, Epstein-Barr virus (EBV), mega virus, Norwalk virus, coxsackie virus, middle east respiratory syndrome-related coronavirus, severe acute respiratory syndrome-related coronavirus, SARS-Cov-2 virus, hepatitis B, varicella zoster virus, parvovirus, adenovirus, Marburg virus, Ebola virus, Rabies, Smallpox, HIV, Hantavirus, Dengue, Rotavirus, MERS-CoV, mumps virus, cytomegalovirus (CMV), Herpes virus, papillomavirus, chikungunya virus, or any combination thereof.

In some embodiments, the genetically engineered human cell is differentiated from a stem cell. In some embodiments, the stem cell is an induced pluripotent stem cell (iPSC), an embryonic stem cell (ESC), an adult stem cell (ASC), a pluripotent stem cell (PSC), or a hematopoietic stem and progenitor cell (HSPC). In some embodiments, the stem cell is an induced pluripotent stem cell (iPSC).

In some embodiments, the genetically engineered human cell is an epithelial cell or endothelial cell. In some embodiments, the genetically engineered human cell is not a cancer cell. In some embodiments, the genetically engineered human cell has been irradiated.

In some embodiments, the immune cell is an innate immune cell. In some embodiments, the innate immune cell is an NK cell, a macrophage, a dendritic cell, a neutrophil, or an eosinophil. In some embodiments, the innate immune cell is an NK cell.

Some embodiments provide a population of genetically engineered human cells as disclosed herein.

Some embodiments provide a pharmaceutical composition comprising the genetically engineered human cell as disclosed herein, and an excipient. Some embodiments provide a unit dosage form comprising a composition or genetically engineered human cell as disclosed herein.

Some embodiments provide a method of making a population of genetically engineered human stem cells, the method comprising: obtaining a population of human stem cells; inducing a genomic disruption in at least one HLA gene or at least one transcriptional regulator of the HLA gene; and introducing a nucleic acid encoding an exogenous cell surface protein that binds to a protein expressed on the surface of a phagocytic or cytolytic immune cell, or a functional fragment or functional variant of the exogenous cell surface protein, wherein the binding results in the activation of phagocytic or cytolytic activity of the immune cell; to thereby produce a population of genetically engineered stem cells.

In some embodiments, the genomic disruption inhibits expression of an HLA protein encoded by the at least one HLA gene on the surface of the cell. In some embodiments, the genomic disruption inhibits expression of an HLA protein encoded by the at least one HLA gene on the surface of the cell for a period of time sufficient to interact with a protein expressed on the surface of an immune cell.

In some embodiments, the at least one genomic disruption is mediated by an endonuclease. In some embodiments, the endonuclease is a CRISPR endonuclease, a Zinc finger nuclease (ZFN), or a Transcription Activator-Like Effector Nuclease (TALEN). In some embodiments, the at least one genomic disruption is mediated by a CRISPR system that comprises an endonuclease and a guide RNA (gRNA), wherein the gRNA comprises an RNA sequence complementary to a DNA sequence of the at least one HLA gene or at least one transcriptional regulator of an HLA gene.

In some embodiments, the genomic disruption is a single strand DNA break or a double strand DNA break.

In some embodiments, the method further comprises introducing a nucleic acid encoding a microbial protein, or an antigenic fragment thereof.

In some embodiments, the microbial protein comprises a nucleocapsid phosphoprotein comprising at least about 85% sequence identity to SEQ ID NO: 54. In some embodiments, the microbial protein is secreted by the genetically engineered human cell, expressed on the surface of the genetically engineered human cell, or expressed within the cytoplasm of the genetically engineered human cell.

In some embodiments, the microbial protein is a viral, bacterial, or parasitic protein. In some embodiments, the microbial protein is a viral protein.

In some embodiments, the stem cells are induced pluripotent stem cell (iPSC), an embryonic stem cell (ESC), an adult stem cell (ASC), a pluripotent stem cell (PSC), or a hematopoietic stem and progenitor cell (HSPC). In some embodiments, the stem cells are induced pluripotent stem cell (iPSC).

In some embodiments, the method comprises differentiating the population of genetically engineered human stem cells. In some embodiments, the cells are differentiated into epithelial cells or endothelial cells.

In some embodiments, the immune cell is an innate immune cell. In some embodiments, the innate immune cell is an NK cell, a macrophage, a dendritic cell, a neutrophil, or an eosinophil. In some embodiments, the innate immune cell is an NK cell.

Some embodiments provide a method of making a population of terminally differentiated genetically engineered human cells, the method comprising: obtaining a population of human stem cells; inducing a genomic disruption in at least one HLA gene or at least one transcriptional regulator of the HLA gene; introducing a nucleic acid encoding an exogenous cell surface protein that binds to a protein expressed on the surface of a phagocytic or cytolytic immune cell, or a functional fragment or functional variant of the exogenous cell surface protein, wherein the binding results in the activation of phagocytic or cytolytic activity of the immune cell, thereby producing a population of genetically engineered human stem cells; and differentiating the population of genetically engineered human stem cells into a population of terminally differentiated genetically engineered human cells.

In some embodiments, the population of genetically engineered human stem cells are differentiated into epithelial cells or endothelial cells.

Some embodiments provide a method of immunizing a human subject against a microbe, the method comprising administering to the subject the genetically engineered human cell as disclosed herein, the composition as disclosed herein, or the pharmaceutical composition as disclosed herein.

Some embodiments provide a method of immunizing a human subject against a microbe, the method comprising administering to the subject a population of genetically engineered human cells comprising: (a) a genomic disruption in at least one HLA gene or at least one transcriptional regulator of an HLA gene; (b) a nucleic acid encoding an exogenous cell surface protein that binds to a protein expressed on the surface of a phagocytic or cytolytic immune cell, or a functional fragment or functional variant of the exogenous cell surface protein, wherein the binding results in the activation of phagocytic or cytolytic activity of the immune cell; and (c) a nucleic acid encoding a microbial protein, or an antigenic fragment thereof.

In some embodiments, the binding results in immune cell mediated lysis or phagocytosis of at least a portion of the population of genetically engineered human cells.

In some embodiments, the administering results in the subject mounting an adaptive immune response against the microbe.

In some embodiments, the administering results in an increase in activation and/or proliferation of T cells that express a T cell receptor that specifically binds the microbial protein or an antigenic fragment thereof.

In some embodiments, the administering results in an increase in activation and/or proliferation of B cells that express a B cell receptor that specifically binds the microbial protein or an antigenic fragment thereof.

In some embodiments, the administering results in an increase in circulating antibodies that specifically bind the microbial protein or antigenic fragment thereof.

In some embodiments, the microbial protein or antigenic fragment thereof, is secreted by the genetically engineered human cell, expressed on the surface of the genetically engineered human cell, or expressed within the cytoplasm of the genetically engineered human cell.

In some embodiments, the microbial protein is a viral, bacterial, or parasitic protein. In some embodiments, the microbial protein is a viral protein. In some embodiments, the viral protein is of a virus of family Coronaviridae. In some embodiments, the viral protein is of a virus of genus Alphacoronavirus, Betacoronavirus, Gammacoronavirus, and Deltacoronavirus. In some embodiments, the viral protein is of a virus of genus Betacoronavirus. In some embodiments, the viral protein is of a virus of species severe acute respiratory syndrome-related coronavirus 2. In some embodiments, the viral protein is of a virus of strain severe acute respiratory syndrome coronavirus 2. In some embodiments, the viral protein is a spike protein of severe acute respiratory syndrome coronavirus 2. In some embodiments, the viral protein is a spike protein of SEQ ID NO: 1. In some embodiments, the viral protein is a spike protein encoded by SEQ ID NO: 53.

In some embodiments, the viral protein is from a virus selected from the group that comprises: influenza, Epstein-Barr virus (EBV), mega virus, Norwalk virus, coxsackie virus, middle east respiratory syndrome-related coronavirus, severe acute respiratory syndrome-related coronavirus, SARS-CoV-2 virus, hepatitis B, varicella zoster virus, parvovirus, adenovirus, Marburg virus, Ebola virus, Rabies, Smallpox, HIV, Hantavirus, Dengue, Rotavirus, MERS-CoV, mumps virus, cytomegalovirus (CMV), Herpes virus, papillomavirus, chikungunya virus, and any combination thereof.

In some embodiments, the population of genetically engineered human cells are administered intramuscularly or subcutaneously.

In some embodiments, the immune cell is an innate immune cell. In some embodiments, the innate immune cell is an NK cell, a macrophage, a dendritic cell, a neutrophil, or an eosinophil. In some embodiments, the innate immune cell is an NK cell.

In some embodiments, the genetically engineered human cells further comprise a suicide gene.

In some embodiments, the microbial protein comprises a nucleocapsid phosphoprotein comprising at least about 85% sequence identity to SEQ ID NO: 54.

Some embodiments provide a method of immunizing a subject, the method comprising administering to the subject a population of genetically engineered mammalian cells comprising: (a) a genomic disruption in at least one MHC gene or at least one transcriptional regulator of an MHC gene, wherein the disruption results in a reduction of activation of T cell proliferation compared to the genetically engineered human cell without the disruption; and (b) a nucleic acid encoding an exogenous cell surface protein that binds to a protein expressed on the surface of a phagocytic or cytolytic immune cell, or a functional fragment or functional variant of the exogenous cell surface protein, wherein the binding results in the activation of phagocytic or cytolytic activity of the immune cell.

In some embodiments, the immunizing is specific for an antigen, and wherein the genetically engineered mammalian cells further comprise a nucleic acid encoding the antigen or a fragment thereof.

In some embodiments, the immunizing is specific for an antigen, and wherein the genetically engineered mammalian cells further comprise the antigen or a fragment thereof.

In some embodiments, the activation results in immune cell mediated lysis or phagocytosis of at least a portion of the population of genetically engineered mammalian cells.

In some embodiments, the administration results in the subject mounting an adaptive immune response against the antigen. In some embodiments, the administration results in an increase in activation and/or proliferation of T cells that express a T cell receptor that specifically binds a peptide of the antigen. In some embodiments, the administration results in an increase in activation and/or proliferation of B cells that express a B cell receptor that specifically binds a peptide of the antigen. In some embodiments, the administration results in an increase in circulating antibodies that specifically bind the antigen.

In some embodiments, the antigen is secreted by the genetically engineered mammalian cell, expressed on the surface of the genetically engineered mammalian cell, or expressed within the cytoplasm of the genetically engineered mammalian cell.

In some embodiments, the antigen is a viral, bacterial, fungal, or parasitic protein. In some embodiments, the viral protein is of a virus of family Coronaviridae. In some embodiments, the viral protein is of a virus of genus Alphacoronavirus, Betacoronavirus, Gammacoronavirus, or Deltacoronavirus. In some embodiments, the viral protein is of a virus of genus Betacoronavirus.

In some embodiments, the viral protein is of a virus of species severe acute respiratory syndrome-related coronavirus 2. In some embodiments, the viral protein is of a virus of strain severe acute respiratory syndrome coronavirus 2. In some embodiments, the viral protein is a spike protein of severe acute respiratory syndrome coronavirus 2. In some embodiments, the viral protein is a spike protein of SEQ ID NO: 1. In some embodiments, the viral protein is a spike protein encoded by SEQ ID NO: 53 or SEQ ID NO: 61.

In some embodiments, the viral protein is from a virus selected from the group that comprises at least one of influenza, Epstein-Barr virus (EBV), mega virus, Norwalk virus, coxsackie virus, middle east respiratory syndrome-related coronavirus, severe acute respiratory syndrome-related coronavirus, SARS-CoV-2 virus, hepatitis B, varicella zoster virus, parvovirus, adenovirus, Marburg virus, Ebola virus, Rabies, Smallpox, HIV, Hantavirus, Dengue, Rotavirus, MERS-CoV, mumps virus, cytomegalovirus (CMV), Herpes virus, papillomavirus, chikungunya virus, or any combination thereof.

In some embodiments, the antigen comprises a protein or peptide associated with a cancer or a tumor. In some embodiments, the antigen comprises a neoantigen.

In some embodiments, the population of genetically engineered cells are administered intramuscularly or subcutaneously.

In some embodiments, the immune cell is an innate immune cell. In some embodiments, the innate immune cell is an NK cell, a macrophage, a dendritic cell, a neutrophil, or an eosinophil. In some embodiments, the innate immune cell is an NK cell. In some embodiments, the genetically engineered mammalian cells further comprise a suicide gene.

In some embodiments, the genetically engineered mammalian cells comprise genetically engineered human cells, and the MHC gene comprises an HLA gene.

Some embodiments provide a genetically engineered human cell comprising: a genomic disruption in at least one human leukocyte antigen (HLA) gene or at least one transcriptional regulator of an HLA gene; and a genomic modification that results in overexpression of at least a portion of a human arrestin domain containing protein 1 [ARRDC1] or a functional variant thereof, wherein the overexpression is in an amount sufficient to result in increased extracellular vesicle formation as compared to a comparable cell that lacks the genomic modification. In some embodiments, the genomic modification comprises an insertion, one or more nucleobase substitutions, or a deletion.

In some embodiments, the insertion comprises an insertion of an exogenous nucleic acid, wherein the exogenous nucleic acid comprises a region that encodes for an arrestin domain containing protein (ARRDC1).

In some embodiments, the exogenous nucleic acid is a DNA or an RNA.

In some embodiments, the exogenous nucleic acid further comprises a region encoding for a nucleic acid binding motif.

In some embodiments, the exogenous nucleic acid encodes a first region comprising an ARRDC1 gene or a functional variant thereof; and a second region comprising a nucleic acid binding motif.

In some embodiments, the exogenous nucleic acid comprises a ARRDC1 gene or a functional variant thereof comprising a sequence that is at least 85% identical to SEQ ID NO: 56.

In some embodiments, the nucleic acid binding motif comprises a transactivator of transcription (Tat) gene.

In some embodiments, the region comprising a nucleic acid binding motif, wherein the nucleic acid binding motif comprises a nucleic acid sequence of that is at least 85% identical to SEQ ID NO: 57.

In some embodiments, the insertion or the deletion is mediated by an endonuclease. In some embodiments, the endonuclease is a CRISPR endonuclease, a Zinc finger nuclease (ZFN), or a Transcription Activator-Like Effector Nuclease (TALEN).

In some embodiments, the exogenous nucleic acid further comprises a region encoding for a microbial RNA. In some embodiments, the genetically engineered human cell further comprises a microbial RNA.

In some embodiments, the microbial RNA is a viral RNA, a bacterial RNA, a fungal RNA, or a parasite RNA. In some embodiments, the microbial RNA encodes for a viral protein. In some embodiments, the microbial RNA is derived from the order Nidovirales. In some embodiments, the microbial RNA is derived from the family Coronaviridae. In some embodiments, the microbial RNA is derived from the subfamily Orthocoronavirinae.

In some embodiments, the microbial RNA is derived from a virus of the genus Alphacoronavirus, Betacoronavirus, Gammacoronavirus, and Deltacoronavirus. In some embodiments, the microbial RNA is derived from a virus of the genus Betacoronavirus. In some embodiments, the microbial RNA is derived from a virus of subgenus Sarbecovirus. In some embodiments, the microbial RNA is an RNA derived from a severe acute respiratory syndrome-related (SARS) coronavirus (CoV). In some embodiments, the severe acute respiratory syndrome-related coronavirus is SARS-CoV2. In some embodiments, the microbial RNA encodes for a spike protein of severe acute respiratory syndrome coronavirus 2. In some embodiments, the microbial RNA encodes for a spike protein, a nucleocapsid protein, or a glycoprotein.

In some embodiments, the spike protein comprises and amino acid sequence that is at least 85% identical to SEQ ID NO: 1. In some embodiments, the spike protein is encoded by SEQ ID NO: 53 or SEQ ID NO: 61.

In some embodiments, the genomic modification comprises an insertion of an exogenous nucleic acid, wherein the exogenous nucleic acid comprises a nucleic acid comprising a region that is derived from a virus; and wherein the virus is selected from a group consisting of: a severe acute respiratory syndrome-related (SARS) coronavirus (CoV), an influenza virus, an Epstein-Barr virus (EBV), a megavirus, a Norwalk virus, a Coxsackie virus, a middle east respiratory syndrome (MERS)-related coronavirus, a SARS-CoV-2 virus, a hepatitis B virus, a varicella zoster virus, a parvovirus, an adenovirus, a Marburg virus, an Ebola virus, a Rabies virus, a Smallpox virus, a human immunodeficiency virus (HIV), a Hantavirus, a Dengue virus, a rotavirus, a MERS-CoV, a mumps virus, a cytomegalovirus (CMV), a Herpes virus, a papillomavirus, a Chikungunya virus, a respiratory syncytial virus (RSV), a variant, or any combination thereof.

In some embodiments, the exogenous nucleic acid further comprises a hairpin RNA. In some embodiments, the hairpin RNA comprises at least one functional region, and wherein the at least one functional region binds to the exogenous nucleic acid. In some embodiments, the hairpin RNA is a TAR. In some embodiments, the TAR comprises a nucleic acid sequence of: SEQ ID NO 56. In some embodiments, the hairpin RNA binds at least a portion of a TAT protein.

In some embodiments, the genetically engineered human cell is a stem cell or an in vitro-differentiated cell. In some embodiments, the in vitro-differentiated cell is differentiated from an induced pluripotent stem cell (iPSC), an embryonic stem cell (ESC), an adult stem cell (ASC), a pluripotent stem cell (PSC), or a hematopoietic stem and progenitor cell (HSPC). In some embodiments, the stem cell is an induced pluripotent stem cell (iPSC), an embryonic stem cell (ESC), an adult stem cell (ASC), a pluripotent stem cell (PSC), or a hematopoietic stem and progenitor cell (HSPC). In some embodiments, the genetically engineered human cell is an epithelial cell, an endothelial cell, or a peripheral blood mononuclear cell (PBMC). In some embodiments, the genetically engineered human cell is not a cancer cell. In some embodiments, the genetically engineered human cell has been irradiated. In some embodiments, a genetically engineered human cell provided herein or a composition provided herein for use in a vaccine.

In some embodiments, the genetically engineered human cell further comprises an exogenous nucleic acid encoding for a cell surface protein that binds to a protein expressed on the surface of a phagocytic or cytolytic immune cell, or a functional fragment or functional variant of the cell surface protein, wherein the binding results in the activation of phagocytic or cytolytic activity of the immune cell.

In some embodiments, the genomic disruption completely inhibits expression of an HLA protein encoded by the at least one HLA gene on the surface of the genetically engineered human cell. In some embodiments, the genomic disruption is in an HLA class I gene. In some embodiments, the HLA class I gene is an HLA-A gene, HLA-B gene, HLA-C gene, or (3-microglobulin gene. In some embodiments, the HLA class I gene is a β-microglobulin gene. In some embodiments, the genomic disruption is in an HLA class II gene. In some embodiments, the HLA class II gene is an HLA-DP gene, HLA-DM gene, HLA-DOA gene, HLA-DOB gene, HLA-DQ gene, HLA-DR gene. In some embodiments, the at least one transcriptional regulator of the HLA gene is a CIITA gene, RFX5 gene, RFXAP gene, or RFXANK gene. In some embodiments, the transcriptional regulator of the HLA gene is a CIITA gene or a B2M gene. In some embodiments, the genetically engineered human cell comprises a genomic disruption in at least one HLA class I gene or the at least one transcriptional regulator of the HLA class I gene and a genomic disruption in at least one HLA class II gene or the at least one transcriptional regulator of the HLA class II gene. In some embodiments, the genetically engineered human cell comprises a genomic disruption in at least one HLA class I transcriptional regulator and a genomic disruption in at least one HLA class II transcriptional regulator.

In some embodiments, the immune cell is an innate immune cell. In some embodiments, the innate immune cell is an NK cell, a macrophage, a dendritic cell, a neutrophil, or an eosinophil. In some embodiments, the innate immune cell is an NK cell. In some embodiments, the binding results in the activation of cytolytic activity of the NK cell. In some embodiments, the cell surface protein is a ligand that specifically binds to a natural killer (NK) cell activating receptor expressed on the surface of an NK cell. In some embodiments, the cell surface protein is selected from the group consisting of: MICA, MICB, ULBP1, ULBP2, ULBP3, ULBP4, ULBP5, ULBP6, CD155, CD112 (Nectin-2), B7-H6, Necl-2, and immunoglobulin Fc. In some embodiments, the cell surface protein is a natural killer (NK) cell activating ligand. In some embodiments, the natural killer cell activating ligand is selected from the group consisting of MICA, MICB, ULBP1, ULBP2, ULBP3, ULBP4, ULBP5, ULBP6, CD155, CD112 (Nectin-2), B7-H6, and Necl-2.

In some embodiments, the genetically engineered human cell comprises an exogenous nucleic acid encoding a secretory protein that binds to a receptor expressed on the surface of a phagocytic or cytolytic immune cell, or a functional fragment or functional variant of the secretory protein, wherein the protein attracts the immune cell towards the genetically engineered human cell.

In some embodiments, the genetically engineered human cell further comprises more than one nucleic acid encoding an exogenous protein, an antigenic fragment thereof, or a suicide gene.

Some embodiments provide a genetically engineered human cell comprising: a genomic modification that results in overexpression of at least a portion of a human arrestin domain containing protein 1 [ARRDC1] or a functional variant thereof, wherein the overexpression is in an amount sufficient to result in increased vesicle formation as compared to a comparable cell that lacks the genomic modification; and an exogenous nucleic acid encoding a cell surface protein, wherein the cell surface protein binds to a protein expressed on the surface of a phagocytic or cytolytic immune cell, or a functional fragment or functional variant of the cell surface protein, wherein the binding results in the activation of phagocytic or cytolytic activity of the immune cell.

In some embodiments, the genomic modification comprises an insertion, a substitution, or a deletion. In some embodiments, the insertion comprises an endonuclease-mediated insertion of an exogenous nucleic acid that encodes at least a portion of an ARRDC1 gene. In some embodiments, the nucleic acid further encodes for at least a portion of a TAT protein.

In some embodiments, the genetically engineered human cell further comprises an exogenous nucleic acid that encodes for a protein or an antigen. In some embodiments, the antigen is a viral antigen. In some embodiments, the antigen comprises an amino acid sequence that is at least 85% identical to SEQ ID NO: 1.

In some embodiments, the exogenous nucleic acid further encodes at least a portion of a TAR nucleic acid.

In some embodiments, the genetically engineered human cell further comprises an exogenous nucleic acid encoding for a protein that binds to an immune cell surface protein, a functional fragment, or a functional variant of the immune cell surface protein. In some embodiments, the immune cell surface protein is on the surface of a phagocytic immune cell or cytolytic immune cell. In some embodiments, the binding results in the activation of a phagocytic immune cell or a cytolytic immune cell. In some embodiments, the phagocytic immune cell is a macrophage, a neutrophil, a dendritic cell, or a B lymphocyte. In some embodiments, the cytolytic immune cell is a T cell or an eosinophil.

Some embodiments provide a population of engineered cells comprising a plurality of the genetically engineered human cell provided herein.

Some embodiments provide a composition comprising (a) a population of extracellular vesicles, wherein the population of extracellular vesicles are produced by the genetically engineered human cell provided herein or the population of engineered cells provided herein; and (b) a pharmaceutically acceptable carrier, wherein the population of engineered cells comprise a microbial RNA.

In some embodiments, the microbial RNA is a viral RNA. In some embodiments, the microbial RNA encodes for a spike protein, a nucleocapsid protein, or a glycoprotein. In some embodiments, the microbial RNA is derived from a severe acute respiratory syndrome-related (SARS) coronavirus (CoV), an influenza virus, an Epstein-Barr virus (EBV), a megavirus, a Norwalk virus, a Coxsackie virus, a middle east respiratory syndrome (MERS)-related coronavirus, a SARS-Cov-2 virus, a hepatitis B virus, a varicella zoster virus, a parvovirus, an adenovirus, a Marburg virus, an Ebola virus, a Rabies virus, a Smallpox virus, a human immunodeficiency virus (HIV), a Hantavirus, a Dengue virus, a rotavirus, a MERS-CoV, a mumps virus, a cytomegalovirus (CMV), a Herpes virus, a papillomavirus, a Chikungunya virus, a respiratory syncytial virus (RSV), a variant, or any combination thereof. In some embodiments, the microbial RNA encodes for a SARS-CoV2 spike protein. In some embodiments, the microbial RNA encodes for a an RSV glycoprotein.

In some embodiments, the pharmaceutically acceptable carrier is an emulsion, a suspension, a polymer, a diluent, a hydrogel, an aqueous sterile injection solution, or anon-aqueous sterile injection solution.

In some embodiments, the population of engineered cells further comprises extracellular vesicles, wherein the extracellular vesicles are isolated from a cell culture of the population of engineered cells.

Some embodiments provide a composition comprising an extracellular vesicle produced by a genetically engineered human cell, the extracellular vesicle comprising: an exogenous nucleic acid, wherein the exogenous nucleic acid encodes for an antigen; and wherein the exogenous nucleic acid is targeted to the extracellular vesicle by a nucleic acid-protein complex comprising: an arrestin domain containing protein 1 (ARRDC1) or a fragment thereof; a nucleic acid binding motif, and a hairpin RNA.

In some embodiments, the nucleic acid binding motif comprises a Tat. In some embodiments, the Tat is operably linked to the hairpin RNA. In some embodiments, the hairpin RNA is a TAR. In some embodiments, the exogenous nucleic acid is linked to the hairpin RNA.

In some embodiments, the nucleic acid-protein complex comprises: (i) an arrestin domain containing protein 1 (ARRDC1) or a fragment thereof; (ii) a Tat; and (iii) a TAR, wherein the TAR is linked to the exogenous nucleic acid.

In some embodiments, the genetically engineered human cell overexpresses at least a portion of ARRDC1 or a fragment thereof. In some embodiments, the genetically engineered human cell is an immune cell, a stem cell, or an in vitro-differentiated cell. In some embodiments, the genetically engineered human cell further comprises a genomic disruption in at least one human leukocyte antigen (HLA) gene or at least one transcriptional regulator of an HLA gene. In some embodiments, the extracellular vesicle comprises a diameter of at least about 40 nanometers (nm). In some embodiments, the extracellular vesicle comprises a diameter of at least about 40 nm up to 1000 nm.

Some embodiments provide a method of inducing an immune response to an antigen in a subject, the method comprising: administering to a subject the population of genetically engineered human cells provided herein, thereby inducing an immune response to an antigen.

Some embodiments provide a method of inducing an immune response to an antigen in a subject, the method comprising: administering to a subject any one of the compositions provided herein, thereby inducing an immune response to an antigen.

In some embodiments, the administering is systemic administration or local administration. In some embodiments, the administering is intravenous administration. In some embodiments, the antigen comprises a viral antigen, a bacterial antigen, a fungal antigen, or a parasite antigen. In some embodiments, the antigen comprises a viral antigen, and wherein the viral antigen comprises at least a portion of a spike protein, a nucleocapsid protein, or a glycoprotein. In some embodiments, the antigen is a SARS-CoV-2 spike protein antigen.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Examples

### Example 1. CRISPR Genetic Engineering of iPSCs to Knockout Both MHC Class I and II Genes

The parental iPS cell line is transfected using the Lonza nucleofection system with Cas9 protein precomplexed with gRNAs targeting genes essential for the expression of MHC class I and II (such as B2M and CIITA). Cells are allowed to recover from transfection and grown in complete growth media for 72 hours before analysis of the targeted loci by PCR and sequencing across the modified region. Loss of MHC I and MHC II is confirmed by surface expression by flow cytometry in iPSC cells stimulated with IFNg (and in differentiated cells generated from these iPS Cells). FIG. 7.

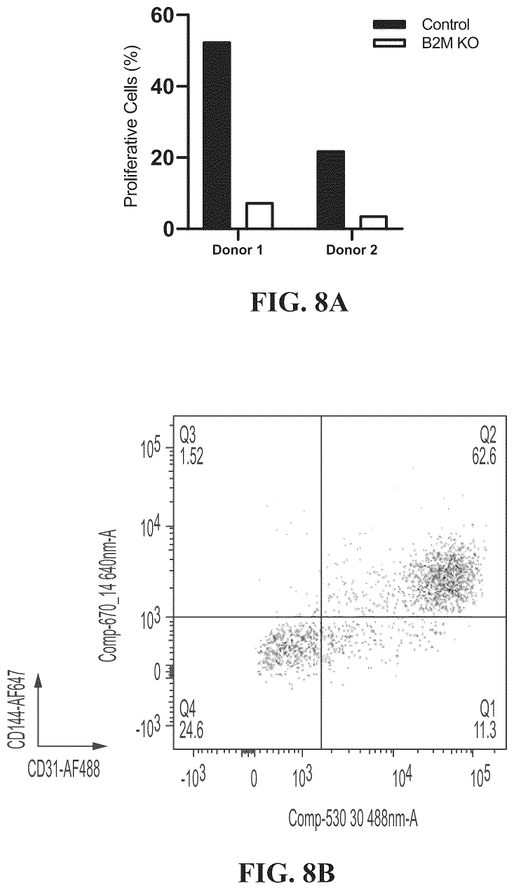

In a further experiment, control cells or B2M knock out platform cells from two different donors are cultured with T cells. MHCI deficient iPSC cells fail to activate the proliferation of MHC-mismatched T cells as compared to control iPS cells, FIG. 8A. Further experiments will measure NK cell killing of modified cells to demonstrate elevated cytolysis in the absence of MHCI.

### Example 2. CRISPR Genetic Engineering of MHC-Null iPSCs from Example 1 to Genomically Integrate Critical NK Cell Activation Ligand Genes or Lytic-Associated Genes

### Activation Ligand Genes

MHC-null iPS cells are transfected with Cas9 and gRNA complexes targeting regions of the genome for targeted integration of either plasmid based DNA donors of rAAV templates carrying transgenes for the stimulation, activation or recruitment of innate immune cells. Target sites include genomic safe harbor sites, or genes that repress or inhibit the stimulation, activation or recruitment of innate immune cells to be inactivated via genomic cleavage and insertion of donor templates. Donor templates are designed to express the cDNAs of the ligands with constitutive promoter and terminator sequences.

### Lysis Genes

MHC-null iPS platform cells are transfected with Cas9 and gRNA complexes targeting regions of the genome for targeted integration of either plasmid based DNA donors of rAAV templates carrying transgenes for the lytic signals recognized by innate immune cells, exemplary signals in Table 6. In addition to a lack of MHC-I, innate immune cells such as NK cells, can be effectively activated to kill target iPS cells by a secondary activating signal in the form of a cell surface ligand that interacts with the NKG2D receptor on the surface of NK cells, such as those in Table 6. Target sites include genomic safe harbor sites, or genes that repress or inhibit the stimulation, activation or recruitment of innate immune cells to be inactivated via genomic cleavage and insertion of donor templates. Donor templates are designed to express the cDNAs of the ligands with constitutive promoter and terminator sequences.

Generation of Endothelial Cells from Platform Cells

To differentiate towards the endothelial lineage iPS cells were grown on Vitronectin coated plates and fed with base media of RPMI of B27(-insulin), Glutamax™ Penicillin/Streptomycin containing on day 0-2 6 μM CHIR99021, 10 ng/ml BMP4, and 100 μg/ml AA2P (stage1), followed by 50 ng/mL VEGF165+20 ng/mL FGF+10 μM SB431542 from days 2-7 (stage 2). Results of Day 7 Flow Cytometry for CD31+CD144+ endothelial cells is shown in FIG. 8B.

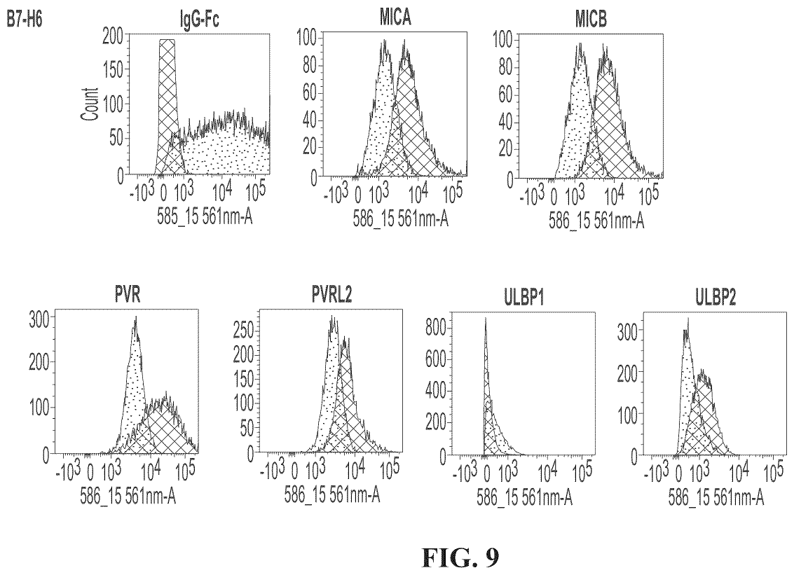

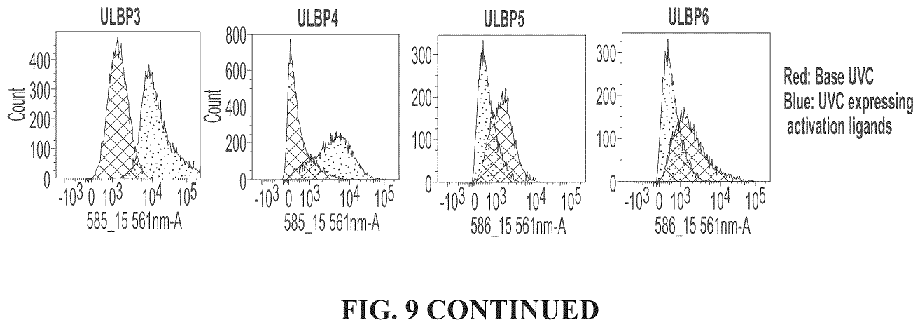

48 hours post-transfection flow cytometry is obtained on iPSC-derived endothelial cells overexpressing NK-activating ligands, data shown in FIG. 9.

TABLE 6
Genes that encode NK cell activating ligands

| Target | Gene | |
|---|---|---|
| 1 | MICA | MICA and MICB molecules act as key ligands |
| 2 | MICB | for activating receptor natural killer cell (NK) |
| | | group 2, member D (NKG2D) and promote NK |
| | | cell-mediated recognition and cytolysis. |
| 3 | PVR | PVR overexpressed on tumor cells increases the |
| 4 | PVRL2 | activation of NK cells and elimination of tumor |
| | | cells via its interaction with DNAM-1 |
| 5 | ULBP1 | ULBP1-6 are recognized by a single NK |
| 6 | ULBP2 | activating receptor, NKG2D |
| 7 | ULBP3 | |
| 8 | ULBP4 | |
| 9 | ULBP5 | |
| 10 | ULBP6 | |
| 11 | CMV pp65 | Viral antigens to be used as a positive control as |
| 12 | B7-H6 | they have been shown to activate NK cells |
| 13 | InfA-HA | |
| 14 | infA-NA | |
| 15 | IgG-FC | “reverse FC” expressed on cell surface to mimic |
| | | an opsonised cell having been coated with |
| | | antibody and thus a target for phagocytosis |

### Example 3. Transfection of the Engineered Cellular Vaccine Cells, Such as Platform Cells, with SARS-Cov-2 Spike Protein or S1 Subunit Expression Constructs with Desired Modification

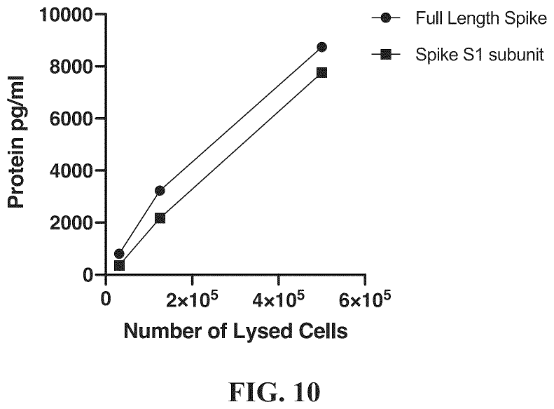

DNA donors (plasmids, linear DNA or rAAV donors) expressing the cDNA for the spike protein or the S1 subunit were transfected into cellular vaccine cells (iPSC or differentiated cells) using Lonza nucleofector, Thermo Neon or any other lipid-based transfection. Endothelial cells expressing the SARS-CoV-2 Spike protein variants were lysed, and lysates analyzed for spike protein antigen by ELISA. Both protein antigen variants could be detected abundantly and showed a dose-dependent increase with vaccine cell number, FIG. 10.

In an exemplary strategy, DNA sequences encoding the Spike antigen variant expression construct are inserted into the AAVS1 safe-harbor site using CRISPR gene engineering.

### Example 4. Coculture of the Transfected Cells in Example 3 with Donor-Derived NK Cells and Analysis of NK-Mediated Cytolysis of the Antigen Loaded Vaccine Cells by Standard Ex Vivo NK-Killing Assays and/or Analysis of Killing by Confocal Imaging

Cellular Vaccine cells are cocultured with PBMC derived NK cells at varying effector:target (E:T) ratios for several days. NK-mediated cell lysis is measured using CyQUANT LDH® Cytotoxicity Assay to measure live and dead cells using a plate reader. NK cell degranulation is also measured by analysis of NK cell CD107a expression by flow cytometry.

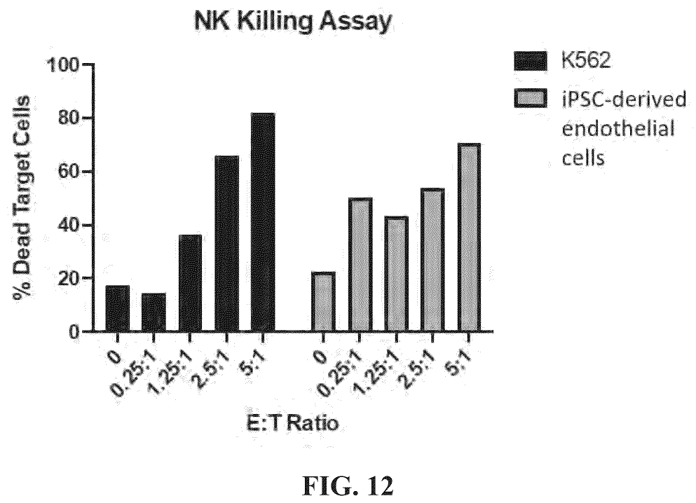

In another assay, 24 hours before performing an NK cell killing assay, iPS derived Endothelial Cells (differentiated from platform cells) were harvested with TRYPLE® and seeded into GELTREX®-coated 96 well plate wells (2×10⁴/well) and incubated overnight in stage 2 endothelial differentiation medium. On the day of the assay K562 cells were seeded out into 96 well plates at (2×10⁴/well) and both endothelial cells and K562 cells were stained with Cell Tracker Blue dye. Primary NK cells were added to the wells at 0; 0.25:1; 1.25:1; 2.5:1; or 5:1 and incubated for 4 hours containing RPMI, 10% FCS with 200 IU/ml IL2 and 10 ng/ml IL15. Samples were then run on a flow cytometer using 7AAD staining to identify dead cells within the Cell Tracker™ Blue target population, FIG. 12.

The data demonstrates effective NK Cell killing in the absence of MHC-I. NK Cell assays measuring the cytolytic killing of MHC-I deficient, iPSC derived Endothelial cells (differentiated from platform cells), show robust, dose dependent lysis that is equivalent to the gold-standard K562 cell line for NK killing. Platform cells or cells differentiated or derived therefrom can be engineered to express NK activating ligands to potentiate this targeted cell lysis further and ensure robust and rapid cytolysis when administered in vivo.

### Example 5. Comparing the Engineered Vaccine Cells with Non-Engineered Cells

NK-mediated killing and degranulation assays are performed as outlined above comparing cellular vaccine cells (iPSC and differentiated cells) to the same cell type without engineering of either ligands to activate the innate immune system or MHC I & II knockout.

### Example 6. Detection of SARS-Cov-2 Spike Protein in the Culture Media Upon NK Cell-Mediated Lysis

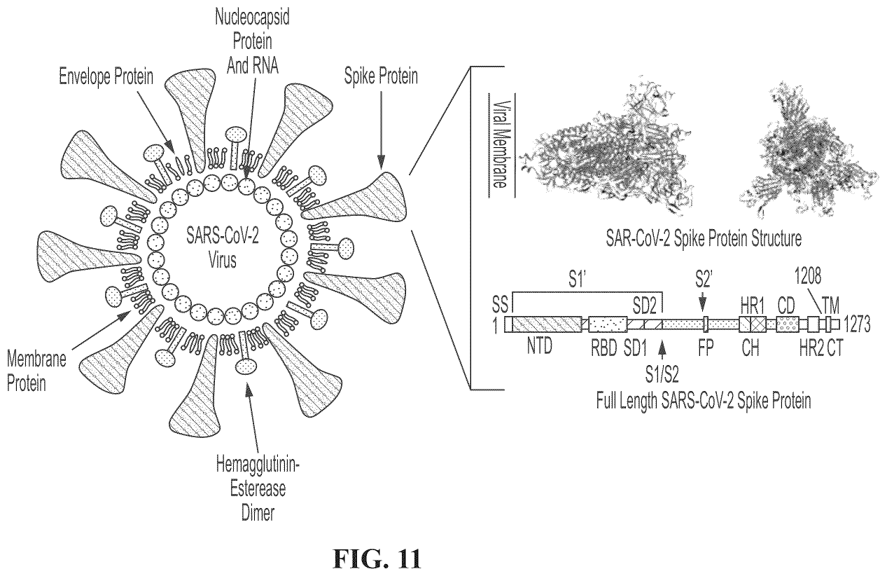

The release of the SARS-Cov-2 spike protein, exemplary schematic at FIG. 11, expressed by the cellular vaccine cells is detected in the supernatant using a Spike protein specific ELISA kit, such as those provided on the world-wide web at <sinobiological.com/elisa-kits/cov-spike-kit40591>.

### Non-Human Primate Study

10 adult rhesus macaques (6-12 years old) are inoculated with a total of 1.1×10⁶ PFU (Group 1; N=3), 1.1×10⁵ PFU (Group 2; N=3), or 1.1×10⁴ PFU (Group 3; N=3) SARS-CoV-2, administered as 1 ml by the intranasal (IN) route and 1 ml by the intratracheal (IT) route. 10 comparable macaques receive control inoculations. Following viral challenge, viral RNA level are assessed by RT-PCR in multiple anatomic compartments, such as bronchoalveolar lavage and nasal swabs.

SARS-CoV-2-specific humoral and cellular immune responses are detected in the animals by evaluating binding antibody responses to the SARS-CoV-2 Spike (S) protein by ELISA and neutralizing antibody (NAb) responses using both a pseudovirus neutralization assay and a live virus neutralization assay. Antibody responses are evaluated against the receptor binding domain (RBD), the prefusion S ectodomain (S), and the nucleocapsid (N). Additionally, the presence of various immune responses are evaluated: antibody-dependent complement deposition (ADCD), antibody-dependent cellular phagocytosis (ADCP), antibody-dependent neutrophil phagocytosis (ADNP), and antibody-dependent NK cell degranulation (NK CD107a) and cytokine secretion (NK MIP1β, NK IFNγ).

### Example 7. A Universal Vaccine Cell (UVC) for SAR-CoV-2

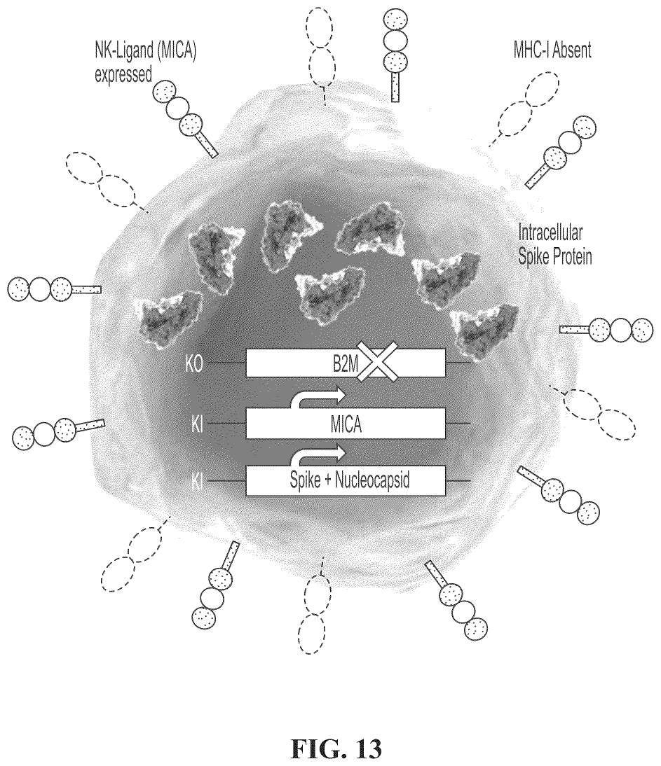

As shown in FIG. 13, the UVC is MHC-I deficient (B2M KO) and does not express MHC-II. The lack of expression of MHC-I potentiates the lysis of the UVC by NK cells. Expression of the NK ligand MICA also further potentiates the NK cell engagement and the UVC cytolysis. The UVC expresses a high level of intracellular SARS-CoV-2 spike protein and nucleocapsid proteins. Upon cell lysis by the NK cell, these proteins are released into the immune microenvironment.

The UVC does not express MHC-II, preventing it from presenting any peptide (e.g., a SARS-CoV-2 spike protein peptide) to any recipient immune cells and unable to be stimulated by IFNγ.

At the site of vaccination, the UVC will activate the innate immune cell (e.g., NK cells) to trigger its own lysis. The spike and nucleocapsid protein will then be released following the apoptosis of the UVC. Phagocytosis and pinocytosis of the UVC apoptotic bodies will enable APCs to present the spike protein and nucleocapsid peptides to the adaptive immune system through MHC presentation. The UVC expresses both a full-length SARS-CoV-2 spike protein and a full-length nucleocapsid protein. To ensure a robust response by the adaptive immune system, the UVC is engineered to express a full-length SARS-CoV-2 spike protein with disrupted furin cleavage sites and two proline residues substitutions. The nucleotide sequence encoding this spike protein is shown in SEQ ID NO: 53 and includes the cDNA of the RSA Spike protein without the furin cleavage site.

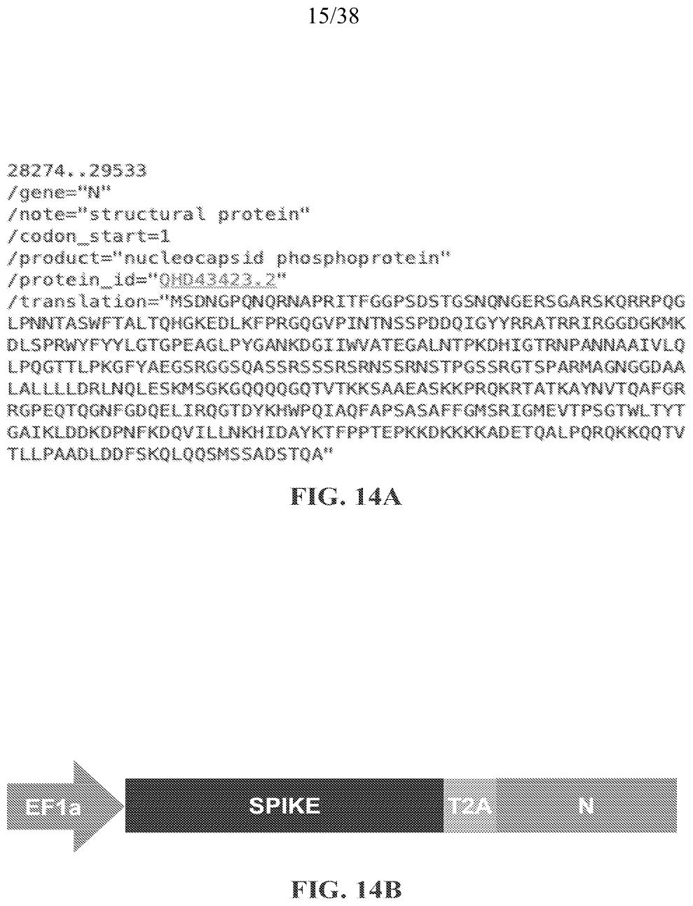

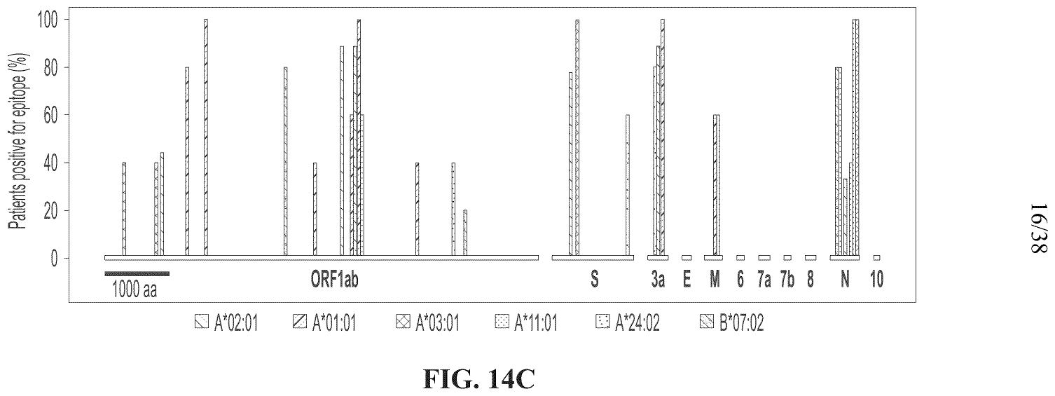

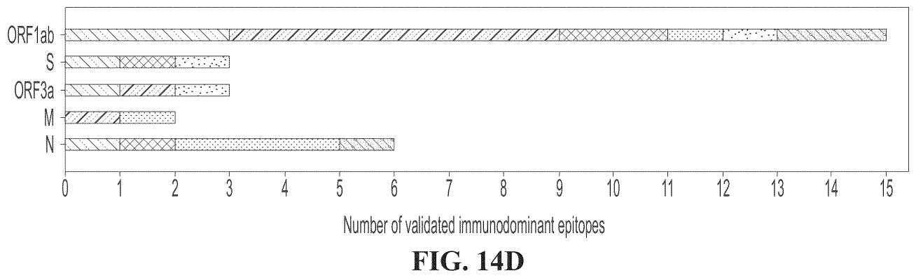

These modifications allow the spike protein to remain intact and a natural cell surface active conformation, because its multiple subunits will dissociate inside the host. The amino acid sequence of the nucleocapsid protein is shown in FIG. 14A or SEQ ID NO: 54.

As shown in FIG. 14B, an EF1a promoter are used to drive and ensure the maximum expression of the SARS-CoV-2 spike protein and the nucleocapsid protein. To maintain a 1:1 expression ratio, the two proteins are expressed from the same transcript with a T2A peptide cleave sequence connecting them.

### Example 8. Polyvalent SARS-CoV-2 UVC Design

A genome-wide screening technology called T-Scan, described in Kula et al., “T-Scan: A Genome-wide Method for the Systematic Discovery of T Cell Epitopes.” 2019, Cell, 178:1016-1028.e3, which is herein incorporated by reference in its entirety for all purposes, was used to determine the global landscape of CD8+ T cell recognition of SARS-CoV-2 in an unbiased fashion: CD8+ T cells were co-cultured with a genome-wide library of target cells (modified HEK293 cells), engineered to express a single HLA allele. Each target cell in the library also expressed a unique coronavirus-derived 61-amino acid (aa) protein fragment. These fragments were processed naturally by the target cell, and the appropriate peptide epitopes were displayed on major histocompatibility complex (MHC) class I molecules on the cell surface. When a CD8+ T cell encountered its target in the co-culture, it secreted cytotoxic granules into the target cell, inducing the apoptosis of its target. Early apoptotic cells were then isolated from the co-culture, and the expression cassettes were sequenced, revealing the identity of the protein fragment. To optimize sorting and isolating rare recognized target cells, the target cells were engineered to express a Granzyme B (GzB)-activated fluorescent reporter as described previously as well as a GzB-activated version of the scramblase enzyme XKR8, which drives rapid and efficient transfer of phosphatidylserine to the outer membrane of early apoptotic cells. Early apoptotic cells were then enriched by magnetic-activated cell sorting with Annexin V, followed by fluorescence-activated sorting with the fluorescent reporter.

A library of 61-aa protein fragments that tiled across all 11 open reading frames (ORFs) of SARS-CoV-2 in 20-aa steps. To capture the known genetic diversity of SARS-CoV-2, all protein-coding variants from the 104 isolates that had been reported as of Mar. 15, 2020 and the complete set of ORFs (ORFeome) of SARS-CoV and the four endemic coronaviruses that cause the common cold (betacoronaviruses HKU1 and OC43 and alphacoronaviruses NL63 and 229E) were included. Known immunodominant antigens from CMV, EBV, and influenza virus were included as positive controls. Each protein fragment with a unique nucleotide barcode to provide internal replicates in our screens was represented 10 times for a final library size of 43,420 clones.

As shown in FIG. 14C, broad reactivity CD8+ T cells to many SARS-CoV-2 proteins, including ORF1ab, S, N, M, and ORF3a, were observed. As shown in FIG. 14D, 3 of the 29 epitopes were located in the spike protein. Most epitopes (15 of 29) were located in ORF1ab, and the highest density of epitopes were located in the N protein. Shared epitopes in the S protein for HLA-A*02:01, HLA-A*03:01, and HLA-A*24:02 but not for HLA-A*01:01, HLA-A* 11:01, or HLA-B*07:02 were observed. Only one recurrent response in the RBD of the S protein (KCY on HLA-A*03:01).

### Example 9: Gene Expression in a CRISPR Engineered UVC

The expression levels of various proteins in the UVC was examined.

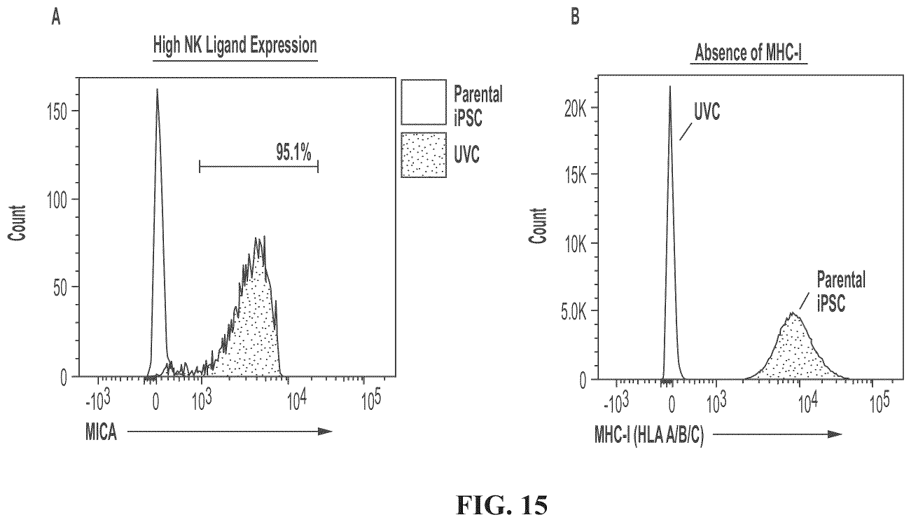

Using CRISPR, a NK ligand MICA was knocked in the UVC genome, and the B2M locus was knocked out to eliminate the expression of MHC-I. Flow cytometry analysis was used to examine the expression level of MICA and MHC-I in the UVC and the parental iPSC. As shown in FIG. 15, most cells in the UVC population showed a high level expression of MICA and minimal expression of MHC-I, compared to the control parent IPSC.

### Example 10: Effective Lysis of UVCs by NK Cells

The UVC can induce effective lysis by the NK cell.

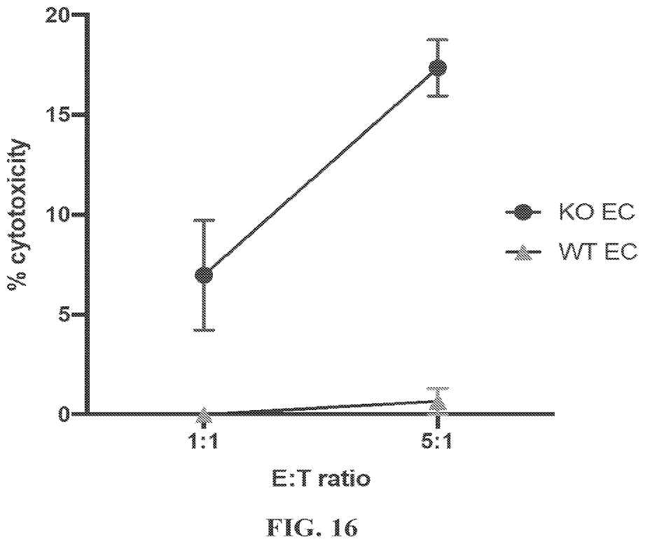

To measure cell lysis of the UVC, a flow cytometry-based NK cytotoxicity assay using Calcein AM (CAM) staining of the NK cells, described in Jang et al., “An Improved Flow Cytometry-Based Natural Killer Cytotoxicity Assay Involving Calcein AM Staining of Effector Cells.” 2012, Ann. Clin. Lab. Sci. Winter; 42(1):42-9, which is herein incorporated by reference in its entirety for all purposes, was used. Macaque NK cells (effector) were stained with CAM and seeded with a fixed number of MHC-I deficient (B2M KO) endothelial cells (EC) as target cells derived from the UVC IPSC. The cells were mixed with an E:T ratio of 1:1 or 5:1. Wildtype ECs were used as a control. Forward scatter profiles with CAM staining were used to distinguish the NK cells and the EC cells. Propidium iodide was used to detect the amount of dead EC cells. The percentage of cytotoxicity was scored as the percentage of dead cells in the total amount of EC cells. As shown in FIG. 16, the NK cell induced an increased amount of lysis in B2M KO-EC than that of WT-EC in either E:T ratio.

Therefore, the UVC can induce effective lysis in vitro by the monkey NK cell.

### Example 11: Additional Responses from NK Cells by NK Ligands

The NK ligand can induce additional responses from the NK cell.

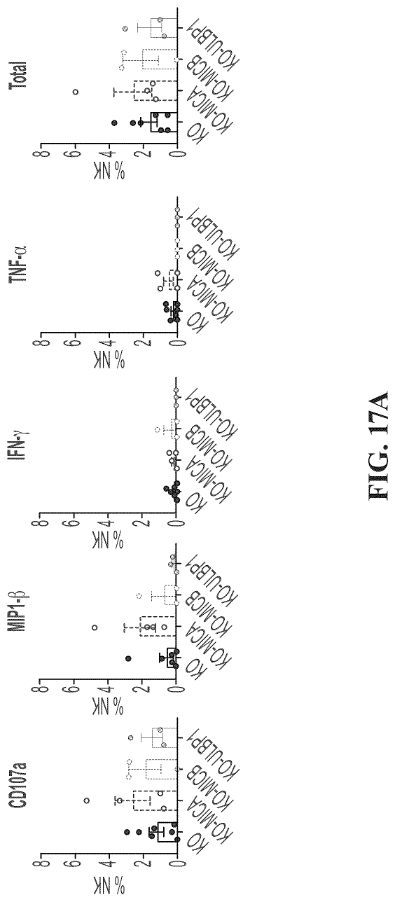

To show that the NK ligand can increase the NK cell, intracellular cytokine staining (ICS) was used to determine the expression of CD107a, MIP1-β, IFN-γ, or TNF-α in an MHC-I deficient UVC (KO), UVC transfected with a MICA expression construct (KO-MICA), UVC transfected with a MICB expression construct (KO-MICB), or UVC transfected with a ULBP1 expression construct (KO-ULBP1). Nucleofection was used to transfect the UVC. For the MICA and MICB construct, the transfection efficiency was about 40 to 70%. The expression construct drove a high level expression of the respective NK ligand in the transfected UVC. As shown in FIG. 17A, KO-MICA increased the total amounts of NK cells with CD107a or MIP1-β expression, while KO-MICA increased the total amounts of NK cells with CD107a expression, compared to that of KO. As illustrated in FIG. 17B, SPICE analysis shows that when responding to the UVC, MICA or MICB also increased the amount of NK cells expressing multiple cytokines. Addition of the NK ligand increases the NK cell response to the MHC-I deficient UVC.

### Example 12: Cell Surface Expression of SARS-CoV-2 Spike Antigens on UVC Cells

The UVC has a robust expression of the SARS-CoV-2 spike protein.

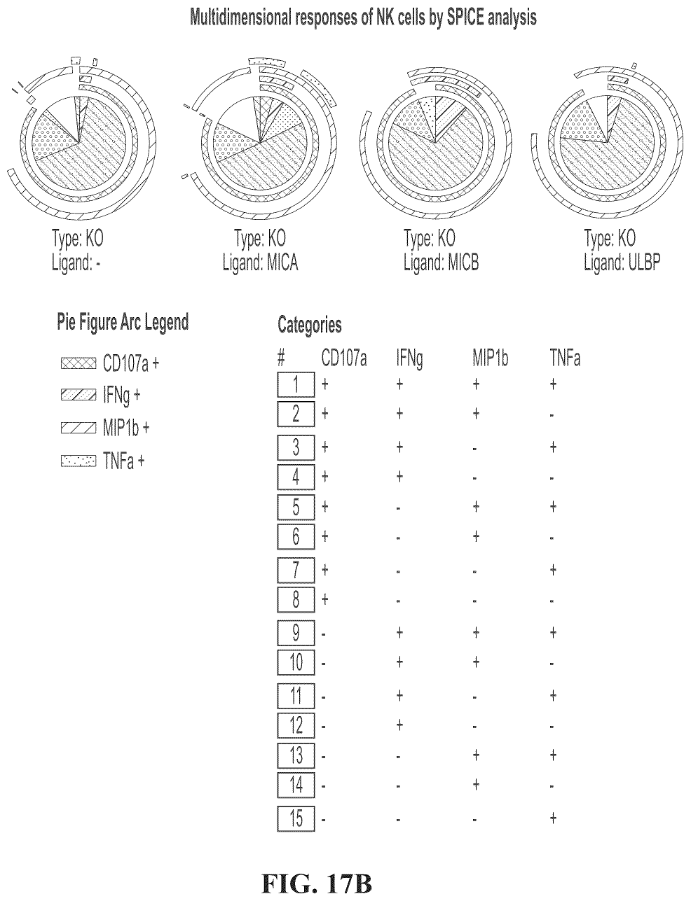

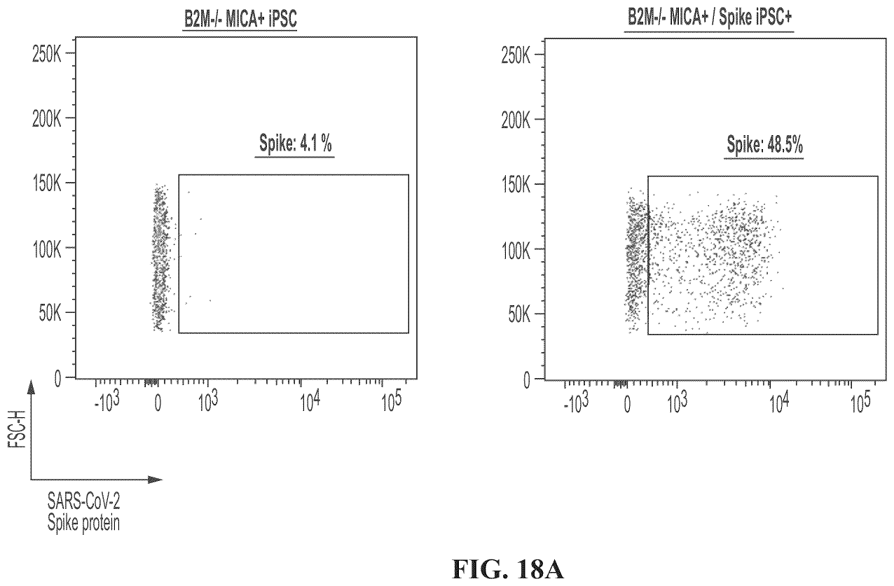

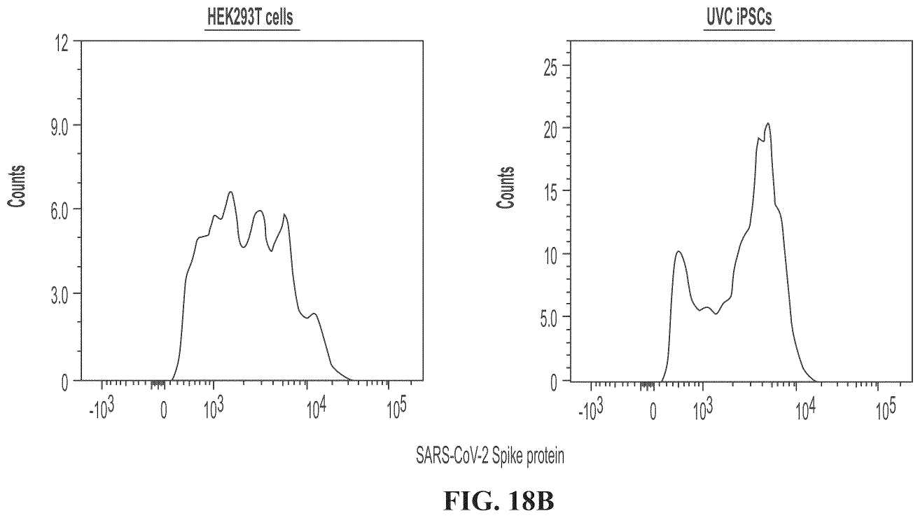

The SARS-CoV-2 spike protein knock-in construct and a MICA knock-in constructed were integrated into the genome of the UVC iPSC with B2M knocked out (B2M−/−). As shown in FIG. 18A, almost half of the engineered UVC iPSC population expressed a high amount of the spike protein. The level of the spike protein expression in the UVC iPSC was similar to HEK293T cells with transient transfection of a spike protein expression construct, as shown in FIG. 18B.

Multivalent antigens (e.g., other SARS-CoV-2 variant spike proteins such as the RSA variant listed in SEQ ID NO: 53; or other proteins such as the nucleocapsid proteins listed in SEQ ID NO: 54) can also be engineered in the UVC.

### Example 13: UVC Non-Human Primate (NHP) Pilot-1 Study

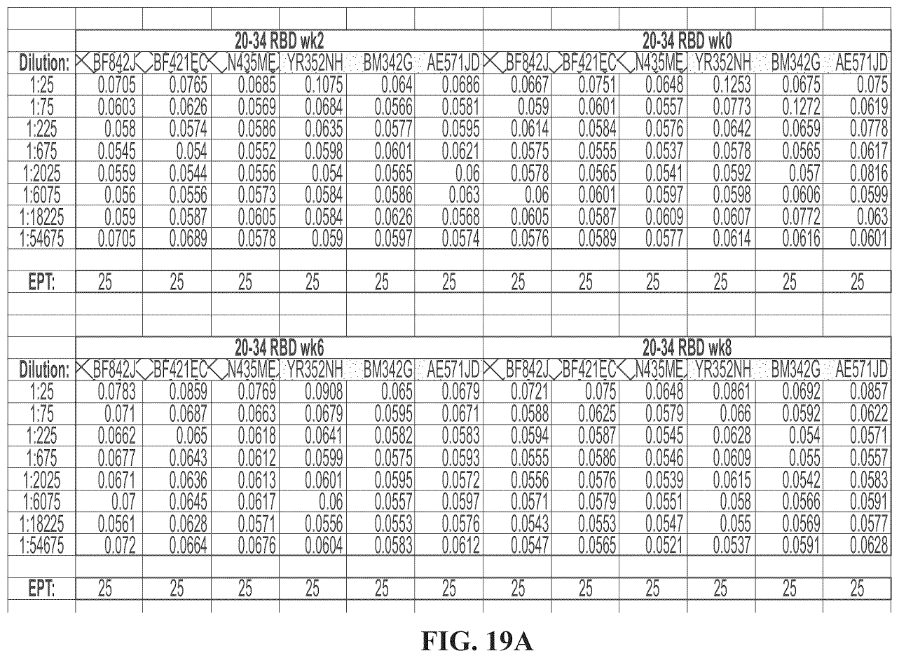

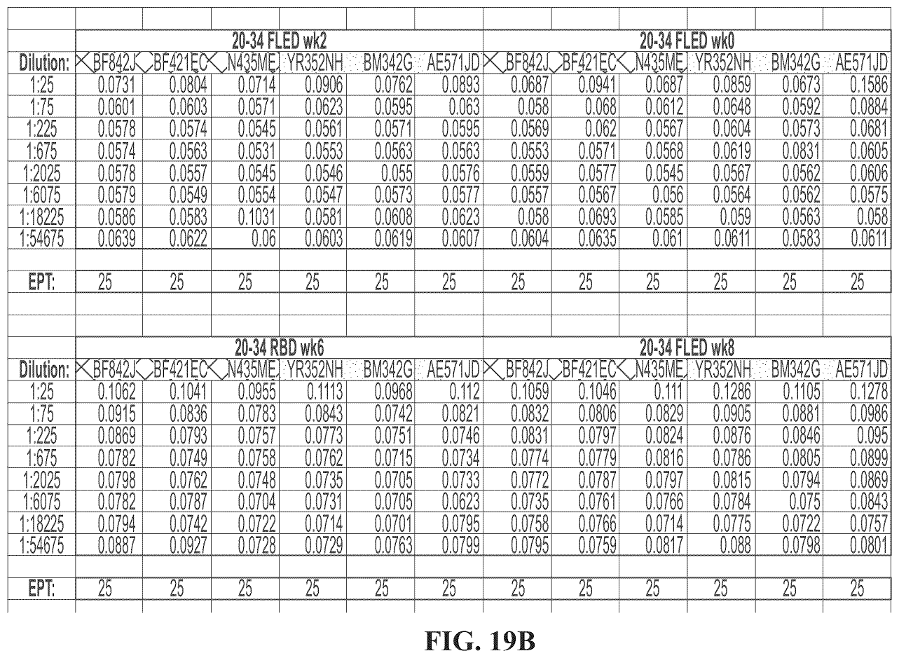

6 monkeys negative for SARS-CoV-2 were administered B2M knock-out, MICA knock-in UVC expressing SARS-CoV-2 spike protein, variant or domain thereof. FIG. 19A and FIG. 19B show the results of antibody ELISA performed at 0, 2, 6, and 8 weeks post vaccination for both the receptor binding domain (RBD) (FIG. 19A) and the full-length SARS-CoV-2 spike protein (FIG. 19B).

RBD-specific and full-length SARS-CoV-2 spike protein-specific binding antibodies were assessed by ELISA as previously described in Chandrashekar, A. et al., Science 369, 812-817 (2020) and Yu, J. et al, Science 369, 806-811 (2020). In brief, 96-well plates were coated with 1 μg ml⁻¹ SARS-CoV-2 RBD or full-length protein (A. Schmidt, MassCPR) in 1×DPBS and incubated at 4° C. overnight. After incubation, plates were washed once with wash buffer (0.05% Tween 20 in 1×DPBS) and blocked with 350 μl casein block per well for 2-3 h at room temperature. After incubation, block solution was discarded and plates were blotted dry. Serial dilutions of heat-inactivated serum diluted in casein block were added to wells and plates were incubated for 1 h at room temperature, before three further washes and a 1-h incubation with a 1:1,000 dilution of anti-macaque IgG HRP (NIH NHP Reagent Program) at room temperature in the dark. Plates were then washed three times, and 100 μl of SeraCare KPL TMB SureBlue Start solution was added to each well; plate development was halted by the addition of 100 μl SeraCare KPL TMB Stop solution per well. The absorbance at 450 nm was recorded using a VersaMax or Omega microplate reader.

### Example 14. Genetic Engineering of iPS Cells to Create a Cellular Vaccine to Deliver the SARS-CoV-2 Spike Antigen

To create a cellular vaccine platform to deliver abundant viral antigens and simultaneously engage host innate immune cells to present these antigens to lymphocytes, a cell with a hyper-immunogenic phenotype was created. Human iPS cells were selected as an embodiment of the UVC cell line due to their stable genetics, non-transformed phenotype, ease of genetic engineering and capacity for rapid scalable propagation. IPS cells also retained the unique ability for programmable differentiated into any cell lineage, thus retaining the future opportunity to explore differentiation of the UVC into different cell types that may have enhanced or different immunogenic properties.

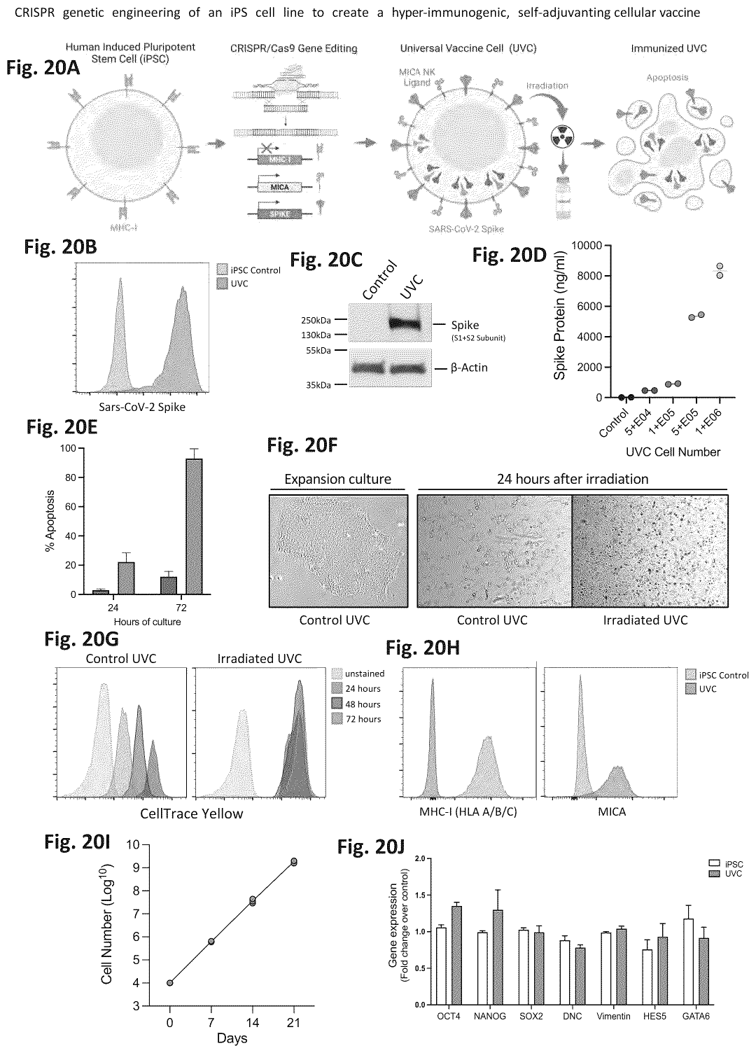

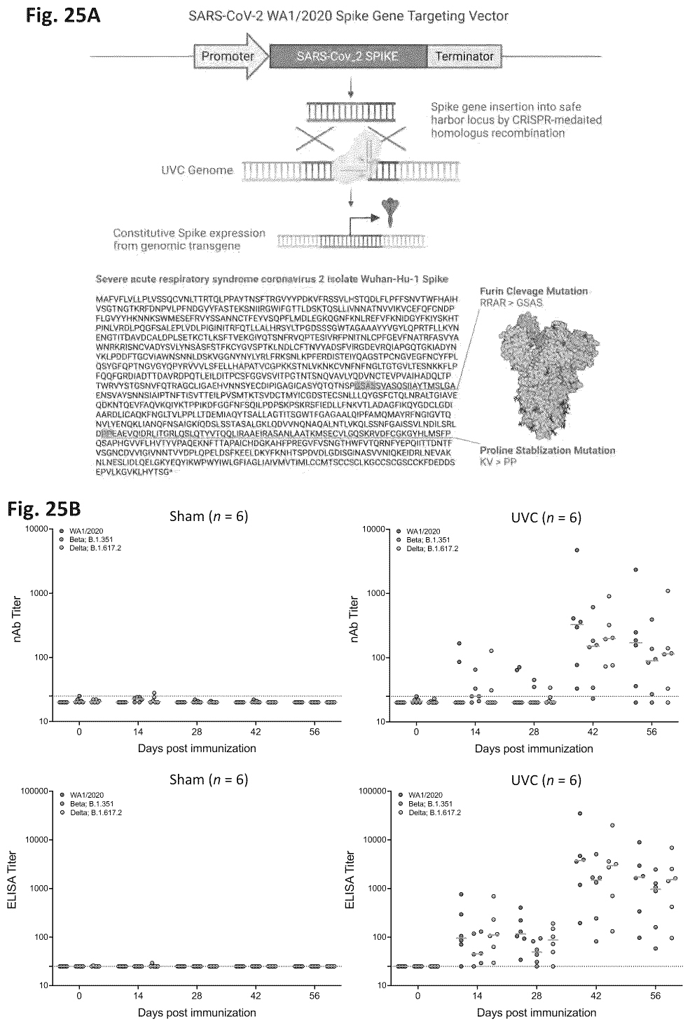

In some embodiments, iPS cells were genetically engineered to create an immunogenic phenotype by stable integration of the SARS-CoV-2 full length spike antigen into the AAVS1 safe-harbor locus using CRISPR/Cas9 gene editing (FIG. 20A). The WA1/2020 variant of SARS-CoV-2 was selected and spike antigen sequence with mutation of the furin cleavage site and proline-stabilizing mutations that is identical to that in the current emergency-use authorized vaccines being deployed globally to vaccinate against COVID-19 (FIG. 25A). By including the spike transmembrane domain sequence in the gene encoding this antigen, high levels of the viral spike were detected on the cell surface of the engineered iPS cells (FIG. 20B). Spike protein was also readily observed when measured in engineered cell lysates by western blotting (FIG. 20C). The yield of antigen released upon lysis was quantified using a spike-specific ELISA assay and an abundant and dose-dependent release of protein from the cells was observed, which would equate to approximately ˜20 micrograms of spike antigen protein delivered in a 1×10⁸ cell vaccine dose of UVC (FIG. 20D)

To ensure robust delivery of this immunodominant antigen to the recipient immune system, an apoptosis-inducing lethal irradiation step was incorporated during vaccine manufacture by exposing the UVC cells to a 10 Gy dose of gamma radiation prior to cryopreservation and vaccination. Thus, in certain embodiments, when subjects are immunized with the UVC, the cells would undergo apoptosis and release the SARS-CoV-2 spike antigen into immune microenvironment via production of apoptotic bodies (FIG. 20A). While not wishing to be bound by any particular theory, it is expected that in some embodiments, these apoptotic bodies will be phagocytosed by innate immune cells and antigen-presenting cells and presented to T and B lymphocytes to generate a spike antigen-specific immune response.

In addition to creating a mechanism for delivery of immunogenic antigens via apoptotic bodies, the irradiation of the UVC can be considered a safety feature as it renders the cells unable to proliferate or persist in vivo upon vaccination. In support of this, a robust elevation in the proportion of apoptotic cells was observed after 24 and 72 hours of culture of irradiated UVC, both using apoptotic dyes and flow cytometry (FIG. 20E) and by observation of cell morphology under the microscope (FIG. 20F). Furthermore, unlike non-irradiated UVC, irradiation prevented any detectible proliferation of the cells over 72-hours in culture as measured by proliferation dyes using flow cytometry (FIG. 20G).

### Incorporation of NK Cell Activation Signals by Genetic Engineering to Create a Self-Adjuvanting Vaccine Cell.

In addition to the immunogenicity expected from apoptosis and release of immunogenic antigens upon vaccination, the immunogenic potential was further increased in some embodiments by incorporating a self-adjuvanting phenotype to the UVC. As a form of physiological cell death, apoptosis is generally non-inflammatory. Therefore, to promote effective local inflammation and engage the innate immune system that can mobilize effector cells, in certain embodiments the UVC was engineered to mimic a virally infected cell to be recognized and rapidly lysed by host innate immune cells, principally NK cells. Many viruses attempt to evade immune recognition by limiting MHC-I cell surface expression to reduce the presentation of viral antigens to CD8+ T cells. This “missing-self” signal can aid in the activation of NK calls and promote cytolysis, and therefore the iPS cells were engineered to completely remove MHC-I molecules from the cell surface via CRISPR knockout of the 02 microglobulin (B2M) gene, a critical component of MHC class I molecules (FIG. 20H).

The UVC was further engineered in a specific embodiment by using CRISPR to integrate a gene expression cassette in a safe-harbor locus to drive constitutive expression of an NK cell activating natural killer group 2 member D (NKG2D) ligand on their cell surface, namely, the human MICA gene (MHC class I polypeptide-related sequence A), a potent activator of NK cells. Using flow cytometry, abundant levels of MICA could be detected on the surface of the engineered UVC (FIG. 20H).

### Rapid Growth Kinetics of Engineered UVC.

Prior to irradiation and cryopreservation of the UVC ready for immunization, the growth kinetics of the cells were evaluated to confirm the capacity for rapid, scalable proliferation that would be needed for a vaccine technology to address the needs of a pandemic. IPS cells are known to have a relatively short doubling times in the range of 18-20 hours, and similar kinetics were observed with an average exponential growth of >50-fold over a 7-day period unrestricted culture (FIG. 20I). Thus, from a starting UVC number of 1×10⁶ cells, the vaccine can be expanded in certain embodiments to provide millions of doses in under 8 weeks, and even quicker if adapted to bioreactor manufacturing.

The consistent rapid cell growth seen with the UVC and their morphological similarities to unmodified iPS cells, suggested they reattained broad characteristic of the iPS cells from which they are derived. The stem cell characteristics of the UVC after genetic engineering and rapid expansion were assessed to confirm that the cells have retained their original stemness-gene expression signatures without acquiring any detectible or obvious changes in phenotype beyond those introduced by genetic engineering. The expanded UVC expressing the SARS-CoV-2 spike antigen, human MICA ligand and CRISPR knockout of B2M, showed a similar level of expression of three important pluripotent transcription factors, NANOG, OCT4 and SOX2, suggesting they have retained a stem-cell like transcriptional profile (FIG. 20J). Engineered UVC also showed similar expression to control iPS cells for genes (DNC, Vimentin, HES5 and GATA6) that are known to increase expression as iPS cells differentiate into mesoderm, endoderm and ectoderm lineage, confirming the UVC have a consistent undifferentiated, iPS cell gene expression profile, morphology, and growth characteristics.

Taken together, these data demonstrate that the engineered UVC as described herein has the capacity to deliver abundant, full-length spike protein antigen in the context of an irradiated, apoptotic cellular vehicle. Via the genetically engineered absence of MHC-I and overexpression of MICA, the UVC has the potential for NK cell activation upon immunization, which removes the need for excipient adjuvants to promote inflammation and immunogenicity. The method was tested in vitro to measure the activation of NK cells by the UVC and the NK-mediated UVC cytolysis.

### Human and Primate NK Cell Cytolysis of Universal Vaccine Cells.

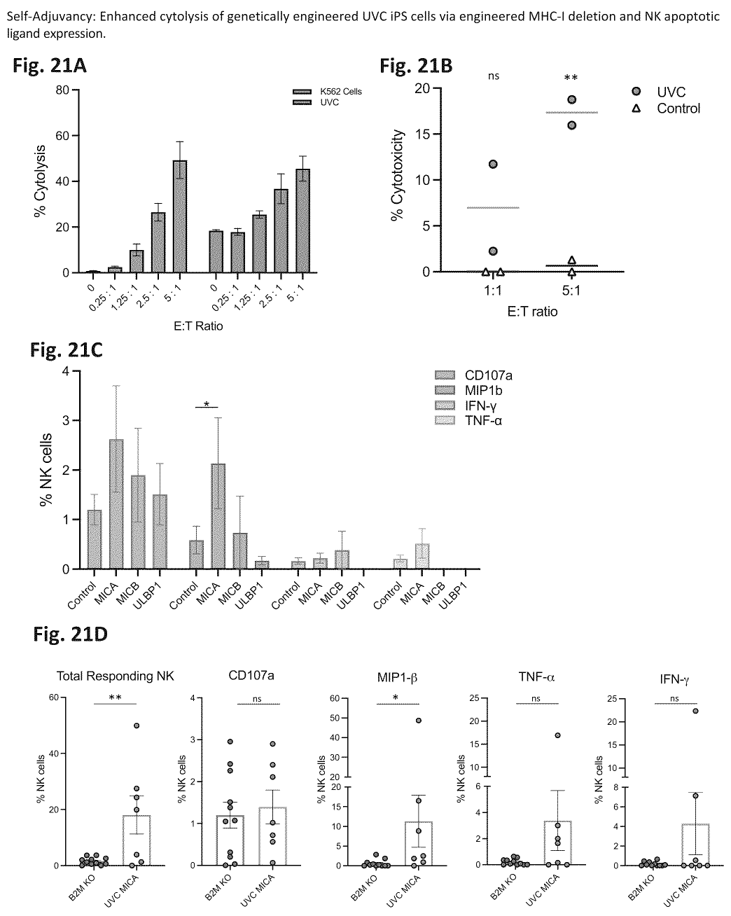

To further explore the impact of MHC-I loss and overexpression of NK cell ligands on recognition and killing of the UVC by NK cells, a series of in vitro NK cell activation and cytolysis assays were performed. When MHC-I was removed via B2M knockout alone, the UVC were robustly killed by human NK cells, which increased in an E:T ratio-dependent manner (FIG. 21A). The level of UVC cytolysis was compared to that observed with the MHC-deficient K562 leukemia cell line, known to be potent targets for NK cell killing, and found a similar level of cytolysis confirming the MHC-I deficient UVC are readily targeted by NK cells. This analysis was extended to macaque NK cells and found that while control iPS cells (expressing MHC-I) show low levels of killing, the MHC-I Knockout UVC were lysed more readily by the NK cells (FIG. 21B).

To assess the relative contribution of overexpressing NK activating ligands on UVC cytolysis by macaque NK cells, an analysis was performed of UVC cells transiently overexpressing different NKG2D ligands, including MICA, MICB and UL16 binding protein 1 (ULBP1). While the levels of macrophage inflammatory protein-1β (MIP-1β) was significantly elevated when MICA was overexpressed, proinflammatory and activation markers for NK cells were generally the same regardless of ligand overexpression (FIG. 21C). With stable overexpression of MICA by CRISPR engineering, a significant increase in total responding macaque NK cells was confirmed.

Collectively these data demonstrate robust cytolysis of the UVC by both human and non-human primate NK cells and show the potent impact of loss of MHC-I on NK cell-mediated lysis.

### Immunogenicity of Universal Vaccine Cells in Vaccinated Macaques.

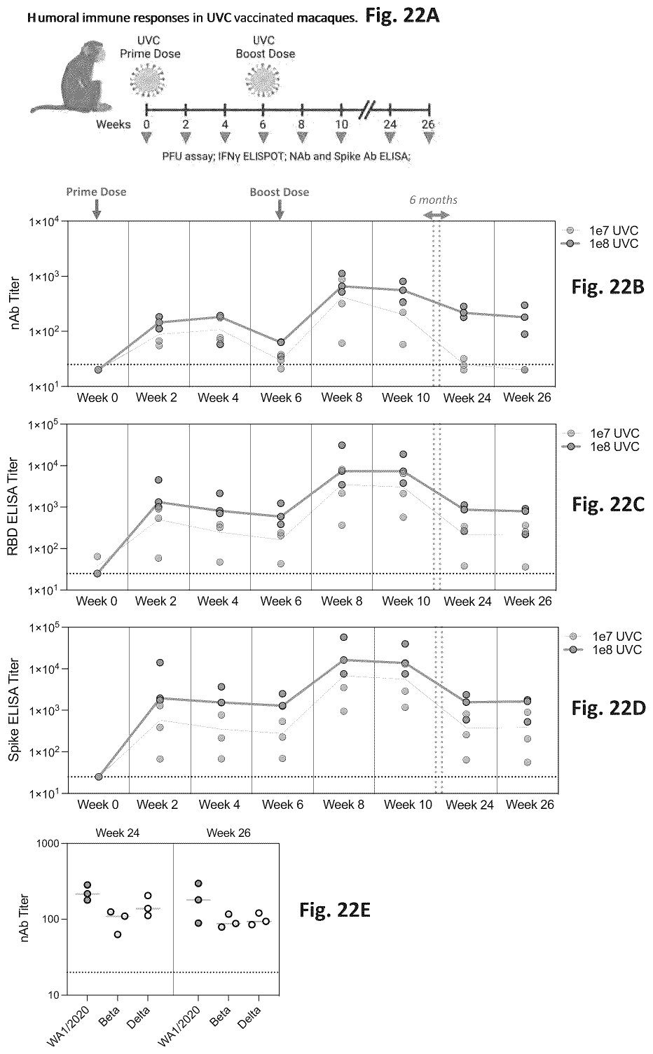

To evaluate the immunogenicity of the UVC and the vaccine's ability to engender a humoral immune response, cynomolgus macaques were immunized and followed the production of neutralizing and spike-specific antibodies over a 10-week period, and a duration follow up at 6 months. 9 macaques, aged 6-12 years old, were immunized with either 1×10⁷ UVC (n=3) or 1×10⁸ UVC (n=3) expressing the WA1/2020 SARS-COV-2 spike antigen, and sham controls (n=3). Macaques received a prime dose immunization by the intramuscular route without adjuvant at week 0, followed by a boost dose immunization (same cell number as prime dose) at week 6 (FIG. 22A). Neutralizing antibody responses were assessed using a pseudovirus neutralization assay, and neutralizing antibodies were observed in all UVC vaccinated macaques at week 2 that further increased by week 4 (FIG. 22B). The higher dose of 1×10⁸ UVC resulted in the most robust titers of neutralizing antibodies at all timepoints tested. Following boost dose immunization with the UVC at week 6, neutralizing antibody titers elevated further, reaching close to 1×10³ titers with the higher 1×10⁸ cell dose. Six months after the initial UVC immunization, neutralizing antibody showed a durable response, and levels in macaques immunized with the 1×10⁸ UVC dose remained elevated beyond that seen with the initial prime UVC dose. Robust spike-specific and receptor-binding-domain (RBD)-specific antibody titers were also observed, as measured by enzyme-linked immunosorbent assay (ELISA) in vaccinated macaques (FIGS. 22C and 22D). These antibody responses and durability at 6 months in the 1×10⁸ dose, were like those seen with the neutralizing antibody titers, and thus collectively demonstrating that the UVC vaccine engenders a robust humoral response against the SARS-CoV-2 spike antigen with demonstrable durability. At 6 months after immunization, detectible levels of neutralizing antibodies against Beta and Delta variants were also observed, suggesting humoral immunity is also generated against SARS-CoV-2 variants (FIG. 22E).

Humoral Immune Responses in Vaccinated Macaques after Heterologous SARS-CoV-2 Challenge.

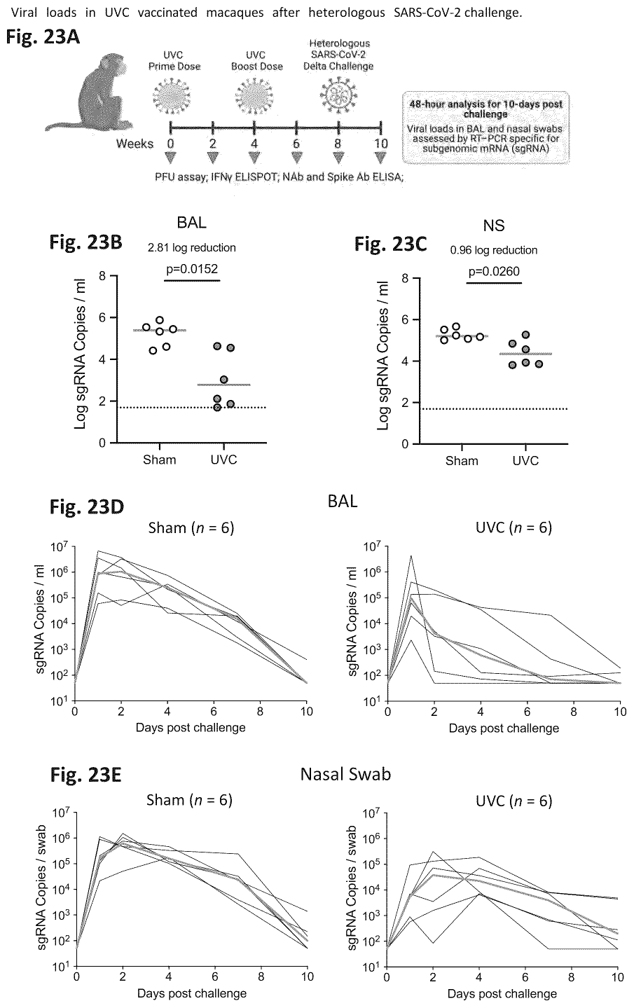

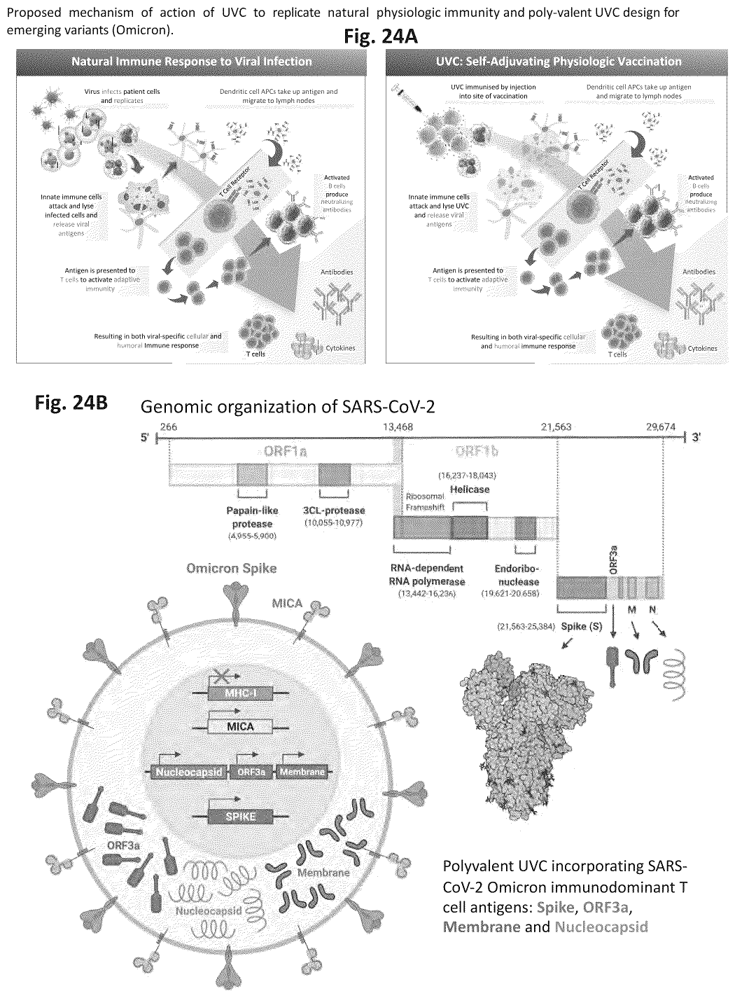

In a second non-human primate study, rhesus macaques, aged 6-12 years old were immunized, with the higher 1×10⁸ dose of UVC (n=6) expressing the SARS-CoV-2 WA1/2020 spike antigen, and sham controls (n=6), and this time followed the production of neutralizing and spike-specific antibodies over an 8-week period (FIG. 23A). At week 8 the macaques were challenged with 1.0×10⁵ 50% tissue culture infectious dose (TCID₅₀) of heterologous SARS-CoV-2 B.1.617.2 (Delta) by the intranasal and intratracheal routes. Viral loads in BAL and nasal swabs were assessed over 10-days by reverse transcription PCR (RT-PCR) specific for subgenomic mRNA (sgRNA), which measures replicating virus. Sham controls showed a median peak of 5.39 (range 4.60-5.88) log₁₀[sgRNA (copies per ml)] in BAL samples (FIGS. 23B and 23D). Partial protection was observed in macaques immunized with the UVC as a significantly lower level of virus was detected in BAL samples, with a median peak of 2.78 (range 1.70-4.63) log₁₀[sgRNA (copies per ml)], representing a 2.81 log reduction in virus in UVC vaccinated animals. A significant reduction in virus (0.96 log reduction) was also observed in nasal swabs from UVC immunized macaques when compared to sham controls, albeit lower in magnitude than seen when comparing BAL samples (FIGS. 23C and 23E).

While neutralizing antibody titers specific for the WA1/2020 variant spike was high in both macaque immunization studies (FIG. 22), the titers specific for other SARS-CoV-2 variants (Beta and Delta) was lower, which is to be expected given the divergence in antigen protein sequence. Thus, the partial protection seen in animals immunized with WA1/2020 spike UVC and challenged with B.1.617.2 (Delta) SARS-CoV-2, is also expected given the heterologous nature of the challenge. In certain embodiments, a robust and complete reduction in virus is predicted from animals immunized with UVC and challenged with SARS-CoV-2 in which the antigen and variant are matched.

Collectively these data demonstrate that a prime and boost dose of 1×10⁸ WA1/2020 Spike expressing UVC promote a robust antigen-specific antibody response with significant levels of neutralizing antibodies and durability, and this can lead to partial protective immunity in a heterologous WA1/2020 versus Delta SARS-CoV-2 virus challenge.

### Example 15. Exemplary Methods Employed in Connection with Example 14

### iPS Cell Culture, Irradiation, and Cryopreservation

Human iPS cells (Thermo Fisher) were cultured on vitronectin-coated T225 cm² flasks using complete mTesSR Plus® medium (StemCell Technologies) supplemented with 1% penicillin/streptomycin, Rock inhibitor (StemCell Technologies) at 1:1000 dilution. For drug selection, G148 was used at 500 μg/ml and puromycin at 5 μg/ml (Sigma-Aldrich). Cultures were maintained at 37° C., 5% CO2 in a humidified incubator. Harvesting of engineered UVC was performed using Accutase™ (StemCell Technologies) and cells were counted using a CellDrop™ cell counter (DeNovix). Cells were irradiated at a total single dose of 10 Gy, before centrifugation at 300×g for 10 minutes followed by resuspension in 100 μl of CryoStor-CS10 freezing media (StemCell Technologies). The UVC preparations for use in non-human primate studies were analyzed for endotoxin levels (Wickham Laboratories Ltd) and absence of Mycoplasma (Mycoplasma Experience Ltd).

### CRISPR Genetic Engineering

CRISPR sgRNAs targeting the human B2M gene, PPP1R12C (AAVS1), and the ROSAβgeo26 locus were designed and validated for indel formation at the selected genomic site. Up to 6 sgRNAs per target gene were tested and the most efficient sgRNA was selected containing 2′-O-methyl and 3′ phosphorothioate modifications to the first three 5′ and the last three 3′ nucleotides (Synthego). 2×10⁶ UVC cells were electroporated using a Neon Nucleofector (Lonza) in Buffer P3 (Lonza) with Cas9 protein (IDT) precomplexed with sgRNA, in a total volume of 100 μl using electroporation program CM138. Gene targeting vectors carrying an expression cassette for expression of human MICA or the SARS-CoV-2 WA1/2020 Spike gene, targeting the Rosa26 and AAVS1 locus respectively, were co-electroporated at 4 μg. Indels introduced by CRISPR editing were detected by PCR and Sanger sequence using DNA primers designed to amplify a 600-900 base pair region surrounding the sgRNA target site. A minimum of 24 hours after electroporation, genomic DNA was extracted using the DirectPCR™ Lysis solution (Viagen Biotech) containing Proteinase K and target regions were amplified by PCR using the GoTaq™ G2 PCR master mix (Promega). Correct and unique amplification of the target regions was verified by agarose gel electrophoresis before purifying PCR products using the QIAquick™ PCR Purification Kit (Qiagen). For analysis by TIDE, PCR amplicons were Sanger sequenced (Eurofins or Genewiz) and paired .ab1 files of control versus edited samples were analyzed using Synthego's ICE tool (available on the world wide web at ice.synthego.com).

### Intracellular Spike Protein Staining

Engineered UVC were harvested and then fixed and permeabilized using BD Cytofix/Cytoperm Fixation/Permeabilization Solution (ThermoFisher). Cells were then stained for intracellular spike protein using an Anti-SARS-CoV-2 Spike Glycoprotein S1 antibody (Abcam, ab275759, 1:50) followed by Goat Anti-Rabbit IgG H&L (Alexa Fluor 488) (Abcam, ab150077, 1:500). Flow analysis was carried out on a Fortessa™ flow cytometer (BD Bioscience), and data analyzed, and flow cytometry figures generated using FlowJo 10 software (BD Biosciences).

### Flow Cytometry Analysis of Cell Surface Antigen Expression

For flow cytometric analysis of cell surface expression of MHC-I, MICA and SARS-CoV-2 spike protein, cells were harvested from culture plates and washed using PBS with 1% Bovine Serum Albumen (Thermo Scientific) and were then stained with PE anti-human MICA/MICB Antibody (6D4, Biolegend), Alexa Fluor 647 anti-human HLA-A,B,C (W6/32, Biolegend), and anti-SARS-CoV-2 Spike Glycoprotein S1 antibody (Abcam, ab275759, 1:50) followed by Goat Anti-Rabbit IgG H&L (Alexa Fluor 488) (Abcam, ab150077, 1:500). Live/Dead Fixable Dead Cell Stains (Invitrogen) were included in all experiments to exclude dead cells. After staining, cells were resuspended in PBS with 2% Human Heat Inactivated AB Serum (Sigma) and 0.1 M EDTA pH 8.0 (Invitrogen) before analysis on a Fortessa™ flow cytometer (BD Bioscience) and data analyzed using FlowJo 10 software (BD Biosciences).

### Western Blot

The SARS-CoV-2 spike glycoprotein was detected in UVC lysates by western blotting. Briefly, cells were lysed by RIPA buffer (20 mM Tris-HCl pH 7.5, 150 mM NaCl, 1 mM EDTA, 0.1% SDS, 1% NP40, 1× protease inhibitor cocktail). Samples were spun at 4° C. for 10 mins at 12,000×g and the pellet discarded. Protein content was measured using BCA Assay (ThermoFisher) using a PHERAstar plate reader (BMG Labtech) at 560 nm. LDS Sample Buffer was added to 30 ng of protein sample to make a 1×solution, with 0.5 μl of beta-mercaptoethanol per well and heated at 70° C. for 10 mins before separation on a polyacrylamide gel (Bio-Rad Mini-PROTEAN TGX Gel 4-15%) and transferred to a PVDF membrane. Membranes were blocked in blocking buffer (5% non-fat powdered milk in TBST), before incubation with primary antibodies in blocking buffer (Rabbit polyclonal anti-SARS-CoV2, Sino Biological 40591-T62, 1:6000 dilution or Mouse b-actin, Abcam 8226, 1 μg/ml), detected with HRP conjugated secondaries in blocking buffer (Goat anti-Rabbit HRP, Sino Biological SSA003, 0.5 μg/ml or Goat anti-Mouse HRP, Abcam ab205719, 1: 4000 dilution) and visualised using the SuperSignal West Femto kit (ThermoFisher) as per kit instructions.

qPCR Measurement of Stem Cell Factors

Total RNA was extracted from UVC cells using the ReliaPrep™ RNA miniprep (Promega) according to the manufacturer's instructions (a DNase treatment was included for all samples), and RNA concentration and absorbance ratios were measured using a Nanodrop One Spectrophotometer (ThermoFisher). cDNA was synthesized using a High-Capacity cDNA Reverse Transcription Kit (the Applied Biosystems) in a total volume of 20 μl to produce DNA that was subsequently assessed by spectrophotometric analysis and diluted to 100 ng/μl. Individual master mixes with each of the DNA-primer combinations for detection of human SOX2, NANOG, OCT4, DNC, Vimentin, HES5 and GATA6 genes were made for 3 replicates using the Brilliant III Ultra-Fast SYBR™ green qPCR master mix (Agilent Technologies) and analyzed on a CFX Opus™ Real-Time PCR system (BioRad) using the following program: 95° C. for 15 minutes for 1 cycle; 95° C. for 15 seconds for 40 cycles; 60° C. for 30 seconds.

### SARS-CoV-2 Spike Protein ELISA

Cells were lysed on ice for 1 hour in water in the presence of protease inhibitors (Sigma). Whole cell lysates were centrifuged at 21,000×g for 10 minutes and the pellet discarded. Spike protein was detected using a SARS-CoV-2 Spike ELISA Kit (Sino Biological) specific for the Spike RBD and the resulting colorimetric change detected using a PHERAstar (BMG LABTECH) plate reader at 450 nm. Protein concentrations in UVC lysates were determined using recombinant SARS-CoV-2 spike protein standards (Sino Biological).

### SARS-CoV-2 Spike Protein ELISA

Cell pellets were harvested and lysed in 20 μl Cell Extraction Buffer (Invitrogen) containing protease inhibitors (Sigma) on ice for 30 minutes, with 3 brief vortexing every 10 minutes. Samples were centrifuged at 13,000 rpm for 10 minutes at 4° C. to pellet insoluble contents. S1 Spike protein was detected using a Covid-19 S-protein ELISA kit (Abcam) specific to S1RBD. Samples were diluted to a range determined to be within the working range of the ELISA kit used and the assay procedure was follows as per manufacturer's instructions. The resulting colorimetric signal was detected at 450 nm using a PHERAstar (BMG LABTECH) plate reader. GraphPad Prism was used to plot a standard curve and interpolate the sample values using a 4-parameter logistic fit.

### UVC Proliferation and Apoptosis Assays

To quantify apoptosis of UVC post-irradiation, cells were stained using a FITC Annexin V Apoptosis Detection Kit with 7-AAD (Biolegend). Proliferation of cells was measured staining of control and Irradiated UVC with either 2 μM Cell Trace Yellow (Abcam) according to kit protocol and analyzing the dilution of the dye at 24-hour periods over 3-days and measuring fluorescence intensity. Flow analysis was carried out on a Fortessa™ flow cytometer (BD Bioscience), and data analyzed, and flow cytometry figures generated using FlowJo 10 software (BD Biosciences).

### CAM Cytotoxicity Assay

Both MHC-I expressing and MHC-I deficient (B2M knockout) UVC were used as target cells for NK cell cytotoxicity assay. Trypsinized cells were stained with calcein acetoxymethyl ester (CAM, Invitrogen) at a 10 μM concentration for 1 h at 37° C. and then washed to remove excess dye. NK cells highly enriched from normal cynomolgus macaque (Macaca fascicularis) blood samples using a CD3 depletion kit (Miltenyi Biotec), were used as effector cells. NK cell effectors and stained target cells were co-cultured in 96 well round bottom plates at effector:target (E:T) ratios of 1:1 and 5:1. Control wells included—only target cells for spontaneous release of CAM and target cells treated with Triton-X 100 for maximum release of CAM. At the end of 4-hour incubation, supernatant was collected for CAM measurement in a fluorescent plate reader at 530 nm. Percent-specific lysis=(test release−spontaneous release)/(maximum release−spontaneous release).

### Nucleofection of NKG2D Ligands in iPS Cells

UVC were cultured in EGM2 (Lonza) media supplemented with 20 ng/mL VEG-F (Peprotech) until 70-90% confluent, in tissue culture flasks pre-coated with sterile 0.1% gelatin in PBS for 1 hour at 37° C. The cells were removed from culture flasks using trypsin, washed, and transfected with plasmid DNA containing either MICA, MICB or ULBP-1 genes after optimizing nucleofection conditions using primary cell 4D nucleofector kit and 4D nucleofector system (Lonza). After 48 hours of culture, transfected cells were stained with aqua dye for live/dead discrimination and corresponding antibodies—MICA/MICB (Clone 6D4, PE, BioLegend) or ULBP-1 (clone 170818, PE, R & D Systems). Stained cells were fixed with 2% paraformaldehyde and acquired on LSRII flow cytometer. Transfection efficiency was calculated as % live cells expressing transfected protein.

### NK Cell Intracellular Cytokine Staining Assay

NK cell effectors were enriched from normal cynomolgus macaque (Macaca fascicularis) blood samples using a CD3 depletion Kit (Miltenyi Biotec). Target and effector cells were plated at E:T ratio of 2:1 in a 96 well round bottom plate. Anti-CD10⁷a antibody (clone H4A3, ECD conjugate, BD Biosciences), brefeldin A and monensin (BD Biosciences) were added to all the samples prior to incubation. After 6 hours of incubation at 37° C., the cells were washed and stained with aqua dye used for live and dead cell discrimination for 20 minutes at room temperature. The cells were then washed and stained for surface markers that included CD3 (SP34.2, BV421, BD Biosciences), CD14 (M5E2, BV650, BD Biosciences), CD16 (3G8, BUV496, BD Biosciences), CD20 (L27, BV570, BD Biosciences), CD56 (NCAM1.2, BV605, BD Biosciences), HLA-DR (G46-6, APC-H7, BD Biosciences) and NKG2A (Z199, PE-Cy7, BD Biosciences) to delineate NK effector cells. Following incubation for 20 minutes, cells were washed and permeabilized using fix & perm reagent (Thermofisher Scientific) as per manufacturer's recommendation. Intracellular cytokine staining was performed for macrophage inflammatory protein 1β (MIP-1β; D21-1351, FITC, BD Biosciences) interferon-γ (IFN-γ; B27, BUV395, BD Biosciences), tumor necrosis factor-α (TNF-α; Mab11, BV650, BD Biosciences) at 4° C. for 15 minutes. Cells were washed, fixed, and acquired on LSRII flow cytometer. Unstimulated NK cells were used for background subtraction of percent positive cells. NK cells stimulated with leukocyte activation cocktail (BD Biosciences) were used as positive control for the assay.

### Animals and Study Design

Outbred Indian-origin adult male and female rhesus macaques (M mulatta), 6-12 years old, were randomly allocated to groups. Macaques were treated with irradiated UVC at doses of either 1×10⁷ or 1×10⁸ cells (n=3-6), and sham controls (n=3-6). Prior to immunization, the cryopreserved doses of irradiated UVC were thawed at 37° C., then 900 μl of 1×PBS was added to each vial of 100 μl UVC in CryoStore freezing media. Macaques received a prime immunization of 1 ml of UVC by the intramuscular route without adjuvant at week 0. At weeks 4 or 6, macaques received a boost immunization of either 1×10⁷ or 1×10⁸ UVC. At week 10 all macaques were challenged with 1.0×10⁵ TCID₅₀ (1.2×10⁸ RNA copies, 1.1×10⁴ PFU) SARS-CoV-2, which was derived from B.1.617.2 (Delta). Viral particle titers were assessed by RT-PCR. Virus was administered as 1 ml by the intranasal route (0.5 ml in each nare) and 1 ml by the intratracheal route. All immunological and virological assays were performed blinded. All animal studies were conducted in compliance with all relevant local, state, and federal regulations and were approved by an Institutional Animal Care and Use Committee (IACUC).

Subgenomic Viral mRNA Assay

SARS-CoV-2 E gene sgRNA was assessed by RT-PCR using primers and probes. In brief, to generate a standard curve, the SARS-CoV-2 E gene sgRNA was cloned into a pcDNA3.1 expression plasmid; this insert was transcribed using an AmpliCap-Max T7 High Yield Message Maker Kit (Cellscript) to obtain RNA for standards. Before RT-PCR, samples collected from challenged macaques or standards were reverse-transcribed using Superscript III VILO (Invitrogen) according to the manufacturer's instructions. A Tagman custom gene expression assay (ThermoFisher Scientific) was designed using the sequences targeting the E gene sgRNA20. Reactions were carried out on a QuantStudio 6 and 7 Flex Real-Time PCR System (Applied Biosystems) according to the manufacturer's specifications. Standard curves were used to calculate sgRNA in copies per ml or per swab; the quantitative assay sensitivity was 50 copies per ml or per swab.

### Serum Antibody ELISA

RBD-specific binding antibodies were assessed by ELISA. In brief, 96-well plates were coated with 1 μg ml-1 SARS-CoV-2 RBD protein (A. Schmidt, MassCPR) in 1×DPBS and incubated at 4° C. overnight. After incubation, plates were washed once with wash buffer (0.05% Tween 20 in 1×DPBS) and blocked with 350 μl casein block per well for 2-3 h at room temperature. After incubation, block solution was discarded, and plates were blotted dry. Serial dilutions of heat-inactivated serum diluted in casein block were added to wells and plates were incubated for 1 hour at room temperature, before three further washes and a 1-hour incubation with a 1:1,000 dilution of anti-macaque IgG HRP (NIH NHP Reagent Program) at room temperature in the dark. Plates were then washed three times, and 100 μl of SeraCare KPL TMB SureBlue Start solution was added to each well; plate development was halted by the addition of 100 μl SeraCare KPL TMB Stop solution per well. The absorbance at 450 nm was recorded using a VersaMax or Omega microplate reader. ELISA endpoint titers were defined as the highest reciprocal serum dilution that yielded an absorbance >0.2. The log₁₀(endpoint titers) are reported.

### Pseudovirus Neutralization Assay

The SARS-CoV-2 pseudovirus expressing a luciferase reporter gene were generated. In brief, the packaging construct psPAX2 (AIDS Resource and Reagent Program), luciferase reporter plasmid pLenti-CMV Puro-Luc (Addgene), and spike protein expressing pcDNA3.1-SARS-CoV-2 SACT were co-transfected into HEK293T cells with calcium phosphate. The supernatants containing the pseudotype viruses were collected 48 hours after transfection; pseudotype viruses were purified by filtration with 0.45-μm filter. To determine the neutralization activity of the antisera from vaccinated macaques, HEK293T-hACE2 cells were seeded in 96-well tissue culture plates at a density of 1.75×10⁴ cells per well overnight. Twofold serial dilutions of heat-inactivated serum samples were prepared and mixed with 50 μl of pseudovirus. The mixture was incubated at 37° C. for 1 hour before adding to HEK293T-hACE2 cells. After 48 hours, cells were lysed in Steady-Glo Luciferase Assay (Promega) according to the manufacturer's instructions. SARS-CoV-2 neutralization titers were defined as the sample dilution at which a 50% reduction in relative light units was observed relative to the average of the virus control wells.

### Statistical Analyses

Statistical differences between two sample groups, where appropriate, were analyzed by a standard Student's two-tailed, non-paired, t-test and between three or more sample groups using two-way or three-way ANOVA using GraphPad Prism 9. Analysis of virological data was performed using two-sided Mann-Whitney tests. Correlations were assessed by two-sided Spearman rank-correlation tests. P values are included in the figures where statistical analyses have been carried out. P values of less than 0.05 were considered significant.

As discussed herein, the UVC vaccine platform can induce robust neutralizing antibody responses in vaccinated macaques when delivering the SARS-CoV-2 WA1/2020 (original variant) full-length membrane-bound Spike protein, with mutation of the furin cleavage site and two proline-stabilizing mutations. It is also demonstrated that the UVC delivering the WA1/2020 Spike antigen enhances neutralizing antibodies and (RBD)-specific binding antibodies specific for the B.1.617.2 (Delta) and B.1.351 (Beta) variants of SARS-CoV-2. This robust and specific humoral response can partially protect rhesus macaques vaccinated with a prime and boost dose of WA1/2020 Spike UVC in a heterologous variant challenge with infectious B.1.617.2 (Delta) SARS-CoV-2 and engender a more rapid clearance of viral RNA in the BAL.

As regards duration of protection, the hyper-immunity postulated by creating a self-adjuvanting, hyper-immune UVC established robust initial nAb titers. When these animals were rechallenged 6-month later, the nAb response stayed robust at the higher, and now established for clinical use, 1e8 dose. Moreover, the persistent nAb response at 6-months remained robust for SARS-CoV-2 WA1/2020, Beta and Delta variants.

As regards intrinsic safety, the UVC of some embodiments undergoes lethal irradiation during manufacture and rapid apoptosis in the immune microenvironment upon vaccination. This is a mechanism of efficacy of the UVC, and a safety feature, by virtue of the impossibility of in vivo persistence and teratogenicity of the cellular antigen carrier. The irradiation-induced apoptosis is further enhanced by CRISPR genetic engineering to remove MHC-I expression and introduce cell surface expression of the NKG2D ligand MICA, making the UVC potent targets for host NK cells. Recruited NK cells will recognize the UVC as virally infected cell through MHC-I absence and MICA activation of NKG2D signaling to mediate a direct killing effect and release of protein antigen. The apoptosis and NK-mediated cytolysis enables the UVC to be a self-adjuvanting vaccine vector, without the need for additional chemicals adjuvants or additional foreign antigens. Thus, the UVC may mimic the physiological engagement of the immune system typical of virally infective cells within the tissues of an individual suffering with the disease.

The use of a living cellular antigen vehicle, as opposed to a lipid nanoparticle or other such inanimate construct, can recapitulate natural immunity without the need for exogenous adjuvants, which may portend greater safety against autoimmune complications.

The genetic engineering, for instance by use of a CRISPR system to render the UVC highly immunogenic and self-adjuvanting, also presents a unique opportunity to address antigen polyvalency. Unlike mRNA or DNA vaccines, or recombinant, replication-incompetent viral vector vaccines, that have a limit on the size of encoded antigen or the number of independent antigens they can deliver, the UVC can be engineered to express and deliver a much higher number of full-length protein antigens. Thus, there is the ability to create polyvalency against multiple epitopes in a rapid modular gene cassette fashion through CRISPR engineering the iPS cell genome.

The UVC is a cell line, and can thus be scaled within appropriate parameters for such a biologic agent. Once pathogen antigens have be genetically engineered, the UVC cell line can be expanded rapidly to scale with predictable growth kinetics and QA/QC controls. The modular nature of the UVC and the ability to integrate emerging viral antigens into the cellular genome using CRISPR, allows scalable manufacture of new polyvalent vaccines to address emerging variants. In fact, the genetic engineering of the UVC cells can be accomplished in a matter of weeks prior to exponential cell culture expansion to create millions of clinical doses. As a test of the rapid and modular manufacturing of the platform, in some embodiments, an engineered polyvalent UVC has been designed against the SARS-CoV-2 Omicron variant (B.1.1.529) (FIG. 24B).

The UVC allows vaccination with multiple SARS-CoV-2 (or other) antigens, including immunodominant T cell epitopes such as those in the nucleocapsid and viral accessory proteins. In the context of COVID-19, additional studies will evaluate the capability of UVCs delivering polyvalent T cell epitopes such as the Nucleocapsid, ORF and Membrane proteins, to generate a CD8 cytolytic and CD4 Helper T cell response in vaccinated macaques to engender duration of protection via T cell amnestic response. Beyond COVID-19, the potential of the theoretically unlimited antigenic payload of a cellular vaccine would allow for “poly-pathogenic polyvalency” and the creation, for example, of a single seasonal respiratory vaccine, which can include influenza, RSV, and pan-coronavirus viral antigens.

In summary, these data establish the first cellular vaccine platform and demonstrate that immunization with a WA1/2020 SARS-CoV-2 Spike expressing UVC vaccine elicits robust neutralizing antibody titers and provided partial protection against heterologous Delta SARS-CoV-2 challenge in rhesus macaques. Establishing this novel cellular vaccine platform technology within the rigorous and timely setting of COVID-19, the UVC may portend a novel class of gene and cell therapy prophylaxis for potential future viral pandemics.

### Example 16. Cellular and Extracellular Vesicle Vaccines

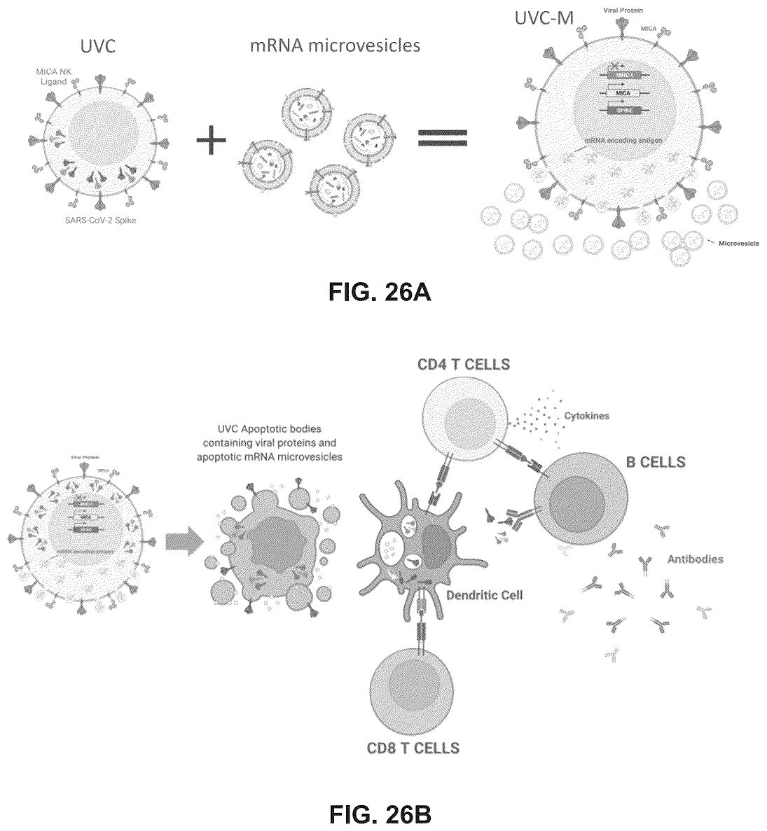

A method of inducing a T cell response is described. The cellular vaccine is generated with mRNA-encoded antigens to enable de novo synthesis of immunodominant epitopes by host cells, in combination with the disclosed genetically engineered and expressed protein antigens. A cellular vaccine is engineered to deliver both endogenously expressed protein antigen (MHC-II) and viral-antigen encoding mRNA (MHC-I) to engender a host cell MHC-II and MHC-I antigen presentation immune response, respectively (FIG. 26A).

To create a UVCs that express both viral antigen proteins (such as SARS-CoV-2 Spike antigen), and incorporates mRNA molecules encoding these viral antigens, cells are CRISPR-engineered to overexpress the human arrestin domain containing protein 1 [ARRDC1], which enables the formation of vesicles within the cell. These vaccines are called UVC-Ms for universal cell-microvesicles Several expression vectors were developed to produce UVC-Ms, including SEQ ID NOS: 57 to 62. Two different isoforms of ARRDC1 were also tested (see, SEQ ID NO: 59 for an expression vector encoding for an ARRDC isoform 1 and SEQ ID NO: 60 encoding for ARRDC isoform 2). The cells are irradiated and transfected with the microvesicle and mRNA producing plasmids.

These arrestin domain containing protein 1 [ARRDC1]-mediated extracellular vesicles (ARMMs) are used for selective recruitment of viral antigen encoding mRNA into the extracellular vesicles within the UVC. The cellular vaccine thus takes the place of the lipid nanoparticle (LNP) as the carrier of the viral antigen-encoding mRNA as currently delivered in current mRNA-based vaccines, including self-amplifying mRNA (SAM).

Packaging of viral antigen RNA into the UVC enables further enhancement of the poly-pathogenic polyvalency of the UVC and introduction of multiple immunogenic epitopes, such as the mRNA encoding the SARS-CoV-2 spike protein, nucleocapsid or ORF antigens into ARMMs. The mRNAs encoding viral antigens into the extracellular vesicles formed within the UVC by ARRDC1, the integrated transgenes expressing these mRNAs are modified to incorporate a signal sequence that can be recognised by a modified ARRDC1 to selectively package the mRNA. The transactivator of transcription (Tat) protein, can bind specifically to a stem-loop containing trans-activating response (TAR) element RNA when incorporated into the viral mRNA molecules.

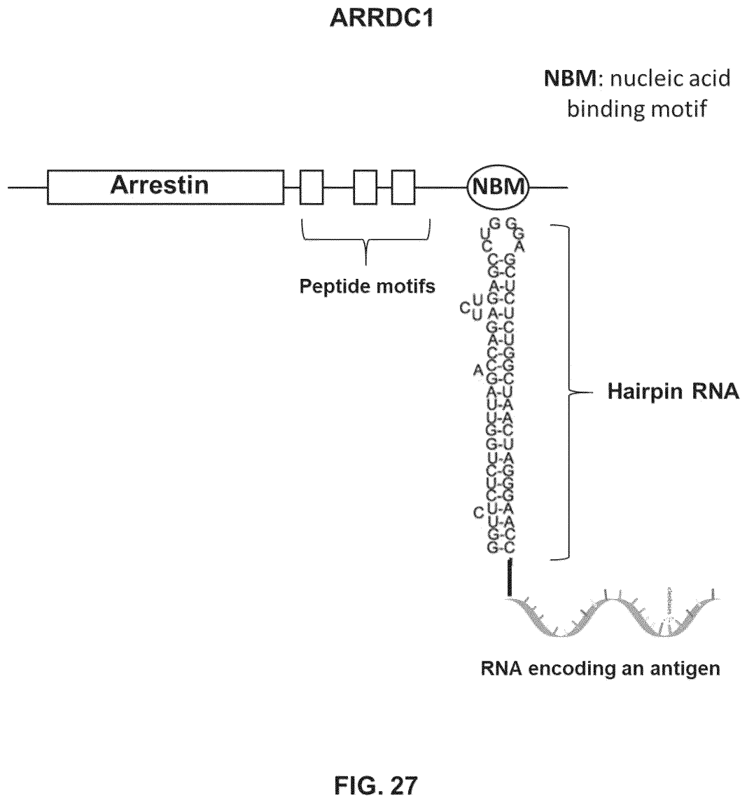

A short nucleic acid binding motif called transactivator of transcription (Tat) peptide is fused directly to the C-terminus of ARRDC1 introduced into the UVC by CRISPR, and another construct with TAR fused directly to the 5′ end of a viral antigen mRNA (FIG. 27). The high binding affinity between Tat and TAR will allow the recruitment of the TAR-fused viral antigen encoding mRNA into ARMMs particles within the UVC.

Upon immunization and lysis of the UVC within the immune microenvironment, both antigen protein and mRNA containing extracellular vesicles will be released into apoptotic bodies which are taken up by host immune cells to encode de novo synthesis antigens from the mRNA (presented as peptide fragments by MHC-I molecules to T cells) as well as the protein antigens released by the UVC presented by MHC-II molecules to B cells, thus potentiating an immune microenvironment and the recruitment of a B cell and T cell lineage as well as compliment (FIG. 26B).

A poly-pathogenic polyvalent universal cellular vaccine is also generated that includes multiple pathogenic viral, bacterial, protozoal, helminthic proteins and other antigenic determinants. For example, a seasonal pan-respiratory virus that can protect against multiple coronavirus variants, multiple influenza variants and respiratory syncytial virus (RSV).

### Example 17. Microvesicle Isolation from Universal Cell Vaccine Cells

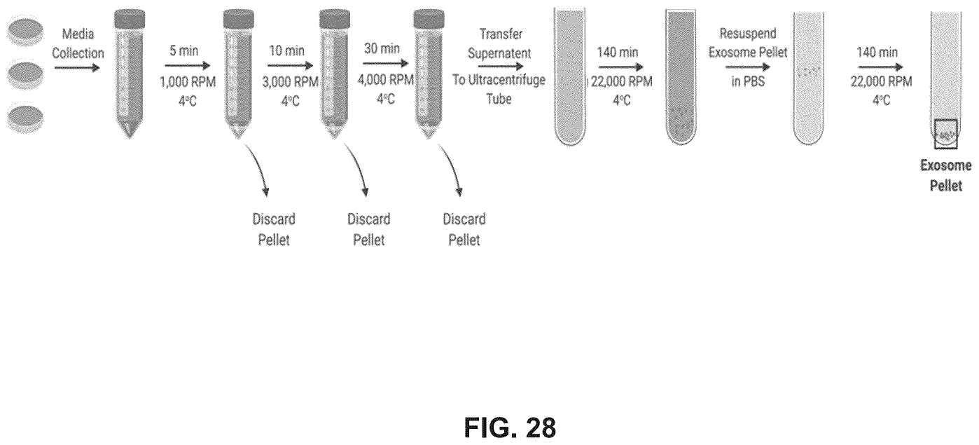

Universal vaccine cells (UVCs) were CRISPR-engineered according to Example 15 to disrupt B2M and were transfected with an expression vector encoding the ARRDC1-TAT-SARS-CoV-2 spike mRNA (SEQ ID NO: 58). One group of cells were irradiated while another group of cells were not irradiated. As a control, cells were not transfected with the ARRDC1-TAT-SARS-CoV2-spike mRNA. Media was collected from each group followed by a series of centrifugation steps as set out below in Table 7 and as outlined in FIG. 28.

TABLE 7
Centrifugation Steps.

| | | rotations | | |
|---|---|---|---|---|
| | | per minute | Temperature | Time |
| Step # | Sample | (RPM) | (° C.) | (minutes) |
| 1 | Media | 1000 | 4° C. | 5 |
| 2 | Supernatant | 3000 | 4° C. | 10 |
| 3 | Supernatant | 4000 | 4° C. | 30 |
| 4 | Supernatant transferred | 22,000 | 4° C. | 140 |
| | to ultracentrifugation | | | |
| | tube | | | |
| 5 | Resuspended exosome | 22,000 | 4° C. | 140 |
| | pellet in phosphate | | | |
| | buffer solution (PBS) | | | |

By step 5, the pellet contained exosomes from UVCs and control human induced pluripotent stem cells (hiPSCs).

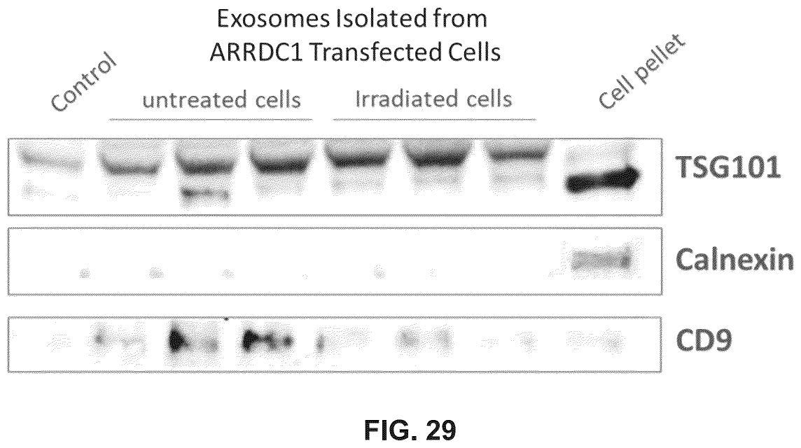

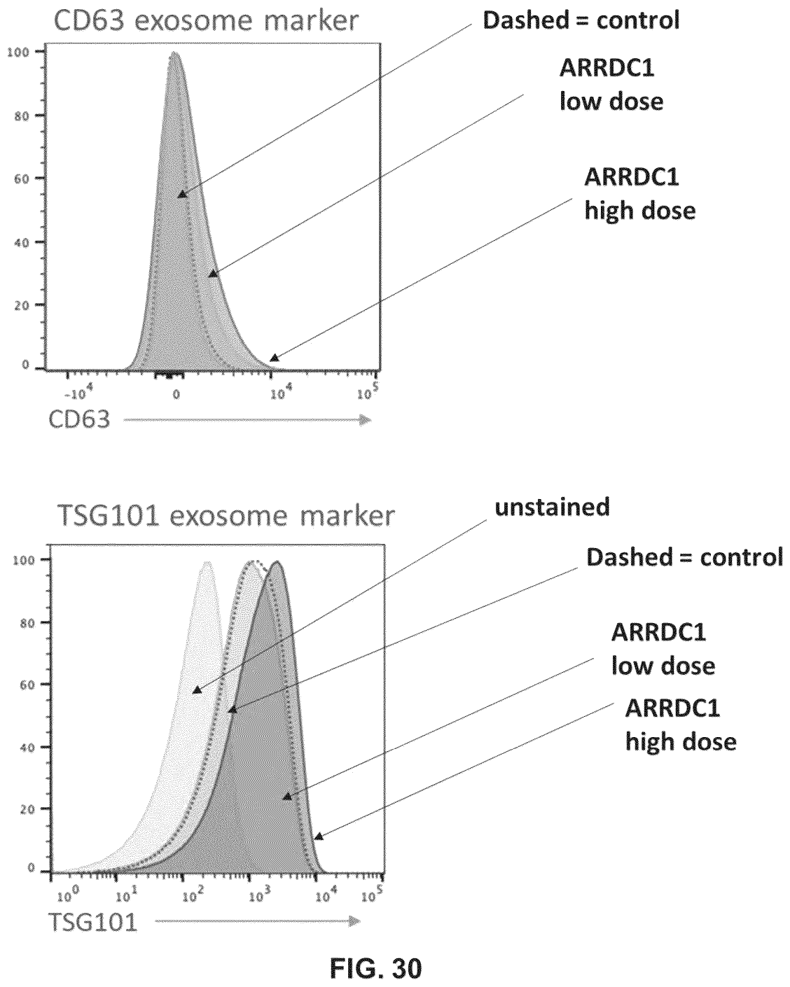

Exosomes were prepared for Western blot analysis and flow cytometry assays. FIG. 29 shows a Western blot of exosome markers, TSG101 and cluster of differentiation 9 (CD9). Calnexin served as a negative marker for exosomes. Both irradiated and non-irradiated cells that were transfected with ARRDC1 expressed TSG101 and CD9, while control cell pellets and cells that were not transfected with ARRDC1 did not express exosome markers. Flow cytometry analysis was performed on isolated exosomes for expression of exosome markers—TSG101 and CD63 (FIG. 30). Cells transfected with ARRDC1 (SEQ ID NO: 58) highly expressed TSG101 relative to control hiPSCs and exhibited an increase in CD63 expression relative to controls,

### Example 18. Spike mRNA Packaging into Microvesicles and Delivery to Recipient Cells

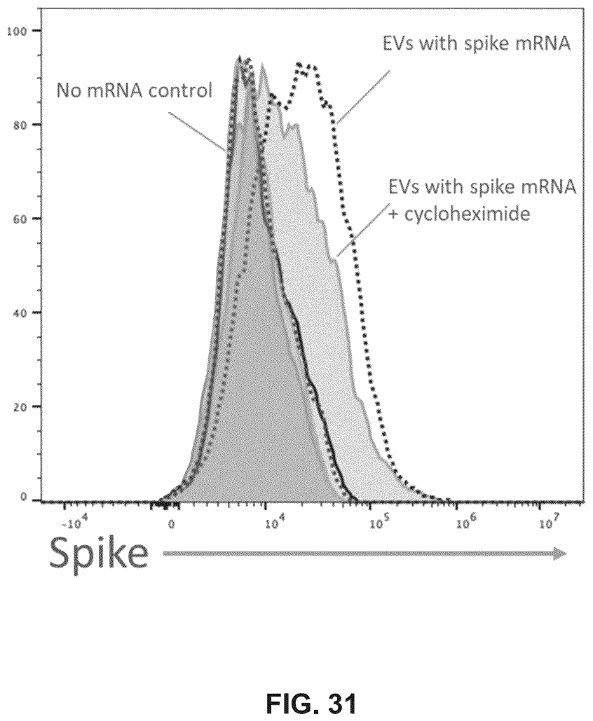

Spike mRNA packaged extracellular vesicles (EVs), e.g., exosomes, were produced by UVC-Ms and isolated as described in Example 17 and flow cytometry for EVs with spike mRNA was performed (FIG. 31). EVs were also treated with cycloheximide as a negative control. as cycloheximide blocks mRNA translation, enabling the measurement of spike antigen produced de novo from mRNA. The cycloheximide treatment was used to demonstrate that the Spike protein expressed and detected on the recipient cell line is produced by de novo protein synthesis of the delivered mRNA (from the vesicles). This is because a drop in signal in the presence of the protein translation blocker cycloheximide can inhibit translation of the mRNA, The assay confirmed that cells transfected with the ARRDC1-spike mRNA construct produced EVs with the spike mRNA.

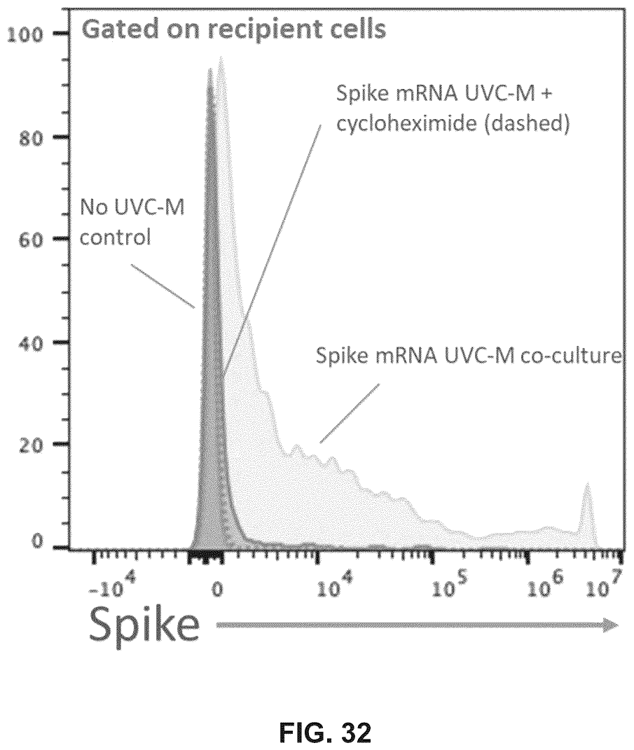

Next, the isolated EVs expressing the spike mRNA were co-cultured with HEK293T recipient cells to drive translation of the spike protein followed by flow cytometry analysis of the recipient cells (FIG. 32). The purified EVs and EVs released by the UVC-M cells were taken up by the recipient cell line and the mRNA was expressed. Recipient cells that were co-cultured with spike mRNA UCV-M EVs expressed the translated protein antigen, SARS-CoV2 spike (S) protein.

### Example 19. UVC-M Cells Deliver mRNA by Microvesicles

UVC-Ms produced according to Example 15 are co-cultured with primary human monocyte-derived macrophages or monocytes for 24 hours. Similarly, microvesicles are also isolated separately from UVCs as in Example 17 for co-culture assays,

UVC-Ms, microvesicles, macrophages, and monocytes are stained for flow cytometry analysis of GFP and SARS-CoV-2 Spike antigen to determine whether phagocytosis of UVC-Ms by the macrophages produce extracellular vesicles and take up the spike antigen. GFP positive macrophages and monocytes indicate that irradiated UVC-Ms or isolated microvesicles successfully deliver mRNA by microvesicles that are phagocytosed and expressed by other cells. The co-culture assay also identifies GFP expression in macrophages and monocytes, as well as intracellular spike protein and MHC-1 spike antigen expression. The expression levels are measured to confirm that microvesicle mRNA has been internalized by macrophages.

Additional assays for evaluating an immune response to a UVC-M or a microvesicle isolated from a UVC includes antigen presentation by other cell types performed by confocal microscopy, functional assays for phagocytosis, cell motility, cell migration assays, and antibody neutralization assays.

1. A genetically engineered human cell comprising:
a genomic disruption in at least one human leukocyte antigen (HLA) gene or at least one transcriptional regulator of an HLA gene; and
an exogenous nucleic acid encoding a cell surface protein that binds to a protein expressed on the surface of a phagocytic or cytolytic immune cell, or a functional fragment or functional variant of the cell surface protein, wherein the binding results in the activation of phagocytic or cytolytic activity of the immune cell,
wherein the genetically engineered human cell is genetically engineered to:
overexpress the human arrestin domain containing protein 1 [ARRDC1], wherein the overexpression is in an amount sufficient to result in at least one of increased intracellular or increased extracellular vesicle formation as compared to a comparable cell that does not exhibit the overexpression of ARRDC1.

2. The genetically engineered human cell of claim 1, wherein the genomic disruption inhibits expression of an HLA protein encoded by the at least one HLA gene on the surface of the genetically engineered human cell.

3. The genetically engineered human cell of claim 2, wherein the genomic disruption completely inhibits expression of an HLA protein encoded by the at least one HLA gene on the surface of the genetically engineered human cell.

4. The genetically engineered human cell of claim 1, wherein the genomic disruption is in an HLA class I gene.

5. The genetically engineered human cell of claim 4, wherein the HLA class I gene is an HLA-A gene, HLA-B gene, HLA-C gene, or β-microglobulin gene.

6. The genetically engineered human cell of claim 5, wherein the HLA class I gene is a β-microglobulin gene.

7. The genetically engineered human cell of claim 1, wherein the genomic disruption is in an HLA class II gene.

8. The genetically engineered human cell of claim 7, wherein the HLA class II gene is an HLA-DP gene, HLA-DM gene, HLA-DOA gene, HLA-DOB gene, HLA-DQ gene, HLA-DR gene.

9. The genetically engineered human cell of claim 1, wherein the at least one transcriptional regulator of the HLA gene is a CIITA gene, RFX5 gene, RFXAP gene, or RFXANK gene.

10. The genetically engineered human cell of claim 9, wherein the HLA gene is a CIITA gene or a B2M gene.

11-21. (canceled)

22. The genetically engineered human cell of claim 1, further comprising more than one nucleic acid encoding an exogenous protein, an antigenic fragment thereof, or a suicide gene.

23. The genetically engineered human cell of claim 22, wherein the exogenous protein comprises a microbial protein.

24-106. (canceled)

107. A method of immunizing a human subject against one or more microbes, the method comprising administering to the subject a population of genetically engineered human cells comprising:
a. a genomic disruption in at least one HLA gene or at least one transcriptional regulator of an HLA gene;
b. a nucleic acid encoding an exogenous cell surface protein that binds to a protein expressed on the surface of a phagocytic or cytolytic immune cell, or a functional fragment or functional variant of the exogenous cell surface protein, wherein the binding results in the activation of phagocytic or cytolytic activity of the immune cell; and at least one of:
c. a plurality of nucleic acids encoding a microbial protein, or antigenic fragments thereof, and
d. a genetic modification to overexpress the human arrestin domain containing protein 1 [ARRDC1].

108. The method of claim 107, wherein the binding results in immune cell mediated lysis or phagocytosis of at least a portion of the population of genetically engineered human cells.

109. The method of claim 107, wherein the administering results in the subject mounting an adaptive immune response against the one or more microbes.

110. The method of claim 107, wherein the administering results in an increase in activation and/or proliferation of T cells that express a T cell receptor that specifically binds the microbial protein or an antigenic fragment thereof.

111. The method of claim 107, wherein the administering results in an increase in activation and/or proliferation of B cells that express a B cell receptor that specifically binds the microbial protein or an antigenic fragment thereof.

112. The method of claim 107, wherein the administering results in an increase in circulating antibodies that specifically bind the microbial protein or antigenic fragment thereof.

113. The method of claim 107, wherein the microbial protein or antigenic fragment thereof, is secreted by the genetically engineered human cell, expressed on the surface of the genetically engineered human cell, or expressed within the cytoplasm of the genetically engineered human cell.

114. The method of claim 107, wherein the microbial protein is a viral protein, a bacterial protein, or parasitic protein.

115-261. (canceled)
